(19) United States
(12) Patent Application Publication
(10) Pub. No.: US 2006/0024308 A1
(43) Pub. Date: Feb. 2, 2006

(54) High affinity anti-TNF-alpha antibodies and method

(76) Inventors: Roberto Crea, San Mateo, CA (US); Arvind Rajpal, Stonington, CT (US); Toshihiko Takeuchi, Oakland, CA (US); Guido Cappuccilli, San Mateo, CA (US)

Correspondence Address: PERKINS COIE LLP, P.O. BOX 2168, MENLO PARK, CA, 94026, US

(21) Appl. No.: 11/176,525

(22) Filed: Jul. 6, 2005

Related U.S. Application Data
(60) Provisional application No. 60/586,487, filed on Jul. 6, 2004.

(51) Int. Cl. C07K 16/24; A61K 39/395

(52) U.S. Cl. 424/145.1; 530/388.23

(57) ABSTRACT

An isolated human anti-TNF-α antibody, or antigen-binding portion thereof, containing at least one high-affinity V_{L} or V_{H} antibody chain that is effective, when substituted for the corresponding V_{L} or V_{H} chain of the anti-TNF-α scFv antibody having sequence SEQ ID NO: 1, to bind to human TNF-α with a K_{off} rate constant that is at least 1.5 fold lower than that of the antibody having SEQ ID NO: 1, when determined under identical conditions.

This application claims priority to U.S. Provisional Patent Application No. 60/586,487 filed on Jul. 6, 2004, which is incorporated herein in its entirety by reference.

### FIELD OF THE INVENTION

The present invention relates to human anti-TNF-α antibodies with enhanced binding activity, and methods of producing and using such antibodies.

### BACKGROUND OF THE INVENTION

Tumor necrosis factor-α or TNF-α is cytokine recognized as the principle mediator of the body's response to gram-negative bacteria. The major source of TNF-α is LPS-activated mononuclear phagocytes, although the cytokine is also produced by antigen-activated T cells, activated NK cells, and activated mast cells (Abbas et al.). At low concentrations, TNF-α has a number of useful biological actions, including promotion of leukocyte accumulation at local sites of inflammation, activation of inflammatory leukocytes to kill microbes, and tissue remodeling, that are critical for local inflammatory responses to microbes. When TNF-α is present at higher concentrations, or under certain immune-response conditions, it can contribute to a variety of pathologies or disorders, including septic shock, autoimmune disorders, graft-versus-host diseases, transplantation rejection, and intravascular thrombosis.

Because TNF-α is associated with several pathological conditions in humans, it has been proposed to treat or ameliorate these conditions in human subjects by administration of a TNF-α antibody. To this end, several groups have reported the development of TNF-α antibodies. The earliest efforts along these lines were aimed at producing mouse monoclonal antibodies specific against human TNF-α (hTNF-α). Although these antibodies displayed high affinity for hTNF-α and neutralized hTNF-α activity, their use in humans was constrained by a number of known limitations associated with administering mouse antibodies to human subjects.

One solution to the limitation of mouse antibodies has been the development of partially humanized antibodies, typically by fusing variable regions of a mouse antibody with the constant regions of a human antibody. Another solution is to derive a fully human anti-TNF-α antibody using human hybridoma cell technology, although the latter approach has yet to produce anti-TNF-α antibodies with binding affinities suitable for therapeutic use. More recently, a fully human-derived TNF-α antibody made by recombinant technology and having binding and neutralization properties suitable for therapy has been reported (see U.S. Pat. Nos. 6,090,382, and 6,509,015).

Despite these advances, there remains a need for anti-TNF-α having enhanced binding affinity properties, e.g., a K_{D} or K_{off} value that is at least 1.5 fold, preferably at least fold, lower than that of the highest affinity TNF-α antibodies available heretofore. Such enhanced-binding antibody would be effective at a substantially lower dose than currently available antibodies and/or would allow for more effective treatment at a comparable dose. These advantages have the potential to reduce the cost and/or improve the therapeutic result in treating a variety of TNF-α associated conditions.

### SUMMARY OF THE INVENTION

The invention includes, in one aspect, an isolated human anti-TNF-α antibody, or antigen-binding portion thereof, containing at least one high-affinity V_{L} or V_{H} antibody chain that is effective, when substituted for the corresponding V_{L} or V_{H} chain of the anti-TNF-α scFv antibody having sequence SEQ ID NO: 1, to bind to human TNF-α with a K_{D} dissociation constant or a K_{off} rate constant that is at least 1.5 fold lower, preferably at least two fold lower, than that of the antibody having SEQ ID NO: 1, when determined under identical conditions.

Exemplary sequences of the antibody V_{L} and V_{H} chains are identified by SEQ ID NOS 2 and 7. Exemplary sequences include those in which least one of the V_{L} CDR1, CDR2, and CDR3 regions may have whose sequence is identified by SEQ ID NOS: 3, 4 and 5, respectively, and in which at least one of the V_{H} CDR1, CDR2, and CDR3 regions whose a sequence is identified by SEQ ID NOS: 8, 9, and 10, respectively.

In a related aspect, the invention includes an isolated human anti-TNF-α antibody, or antigen-binding portion thereof, having V_{L} and V_{H} antibody chains whose sequences are identified by SEQ ID NOS 2 and 7, respectively. Exemplary sequences and embodiments are as noted above.

In another aspect of the invention, there is provided a method of treating a condition that is aggravated by TNF-α activity in a mammalian subject. In practicing the method, the above enhanced-affinity human anti-TNF-α antibody, or antigen-binding portion thereof is administered to the subject, in an amount sufficient to improve the condition in the subject. Exemplary sequences or embodiments of the antibody are as described above.

Also disclosed is a method of identifying human anti-TNF-α antibodies with enhanced binding affinity. In practicing the method, the amino-acid sequence variations contained in the SEQ ID NOS: 2 and 7 for the V_{L} and V_{H} CDRs, respectively, of the anti-TNF-α antibody defined by SEQ ID NO: 1, are used in constructing a library of antibody coding sequences encoding both V_{H} and V_{L} chains of the antibody. The library of coding sequences may include:

(a) a combinatorial library of coding sequences that encode combinations of the V_{L} and V_{H} CDR amino-acid sequence variations contained in at least one of the V_{L} or V_{H} sequences specified by SEQ ID NO: 2 or SEQ ID NO: 7,

(b) a walk-through mutagenesis library encoding, for at least one of the CDRs, the same amino acid substitution at multiple amino acid positions within that CDR, where the substituted amino acid corresponds to an amino acid variation found in at least one amino acid position of the V_{L} or V_{H} sequences specified by SEQ ID NO: 2 or SEQ ID NO: 7, for that CDR, or

(c) a library of localized saturation mutation sequences encoding, for at least one of said CDRs, all 20 natural L-amino acids at an amino acid position that admits to a sequence variation in at least one V_{L} or V_{H} sequences specified by SEQ ID NO: 2 or SEQ ID NO: 7.

The library of coding sequences is expressed in an expression system in which the encoded anti-TNF-α antibodies are expressed in a selectable expression system, and those antibodies having the lowest K_{D} (or EC₅₀) or K_{off} rate constants for human TNF-α are selected.

The library of coding sequences may constructed by identifying amino acid positions that are invariant within one or more selected CDRs, and retaining the codons for the invariant amino acid in the library antibody coding sequences.

The library of coding sequences may be a combinatorial library of coding sequences constructed by (i) producing a primary library of coding sequence encoding antibodies a single amino acid variation contained in at least one of the V_{L} or V_{H} sequences specified by SEQ ID NO: 2 or SEQ ID NO: 7, and (ii) shuffling the coding sequences in the primary library to produce a library of coding sequences having multiple amino acid variations contained in at least one of the V_{L} or V_{H} sequences specified by SEQ ID NO: 2 or SEQ ID NO: 7.

In a related embodiment, the library of coding sequences is a combinatorial library of coding sequences constructed by generating coding sequences having, at each amino acid variation position, codons for the wildtype amino acid and for each of the variant amino acids. In this embodiment, the CDR1-CDR3 coding regions of the library of coding sequences for the V_{L} chain may have the sequences identified by SEQ ID NOS: 11-13, respectively. The CDR1-CDR3 coding regions of the library of coding sequences for the V_{H} chain may have the sequences identified by SEQ ID NOS: 14-16, respectively.

The library of coding sequences may be constructed to encode multiple positively charged amino acids in the CDR-L1 domain or multiple polar amino acids in the CDR-H3 domain.

The expression system employed in the method may be a yeast expression system, and the library of coding sequences may encode scFv anti-TNF-α antibodies.

The library of coding sequences may include, for the CDR1, CDR2, and CDR3 regions of the V_{L} chain, the sequences identified by SEQ ID NOS: 11-13, respectively, and those for the CDR1, CDR2, and CDR3 regions of V_{H} chain may incorporate the sequences identified by SEQ ID NOS: 14-16, respectively. The antibody may be expressed in a scFv format, the expression system employed may be a yeast expression system, and the selection of high-affinity antibodies may be based on a kinetic selection to select antibodies on the basis of enhanced K_{off} binding constants.

In another aspect, the invention includes sequences selected from the group consisting of SEQ ID NOS: 11-16, for use in constructing coding sequences for generating human anti-TNF-α antibodies having one or more of the amino acid substitutions in the V_{L} and V_{H} CDR regions of mutations identified in SEQ ID NOS: 2 and 7, respectively.

These and other objects and features of the invention will become more fully apparent when the following detailed description of the invention is read in conjunction with the accompanying drawings.

### BRIEF DESCRIPTION OF THE DRAWINGS

FIGS. 1A and 1B show the arrangement of variable light-chain (V_{L}) and variable heavy chain (V_{H}) CDRs in a synthetic scFv anti-TNF-α antibody gene (1A) and illustrate the application of look-through mutagenesis (LTM) for introducing a leucine amino acid at each of the fourteen residues 56-69 in the V_{H} CDR2 region of the antibody;

FIG. 2 shows minimum codon base changes needed to produce a Gly-His substitution at a selected codon in walk-through mutagenesis (WTM);

FIG. 3A-3D illustrate minimum codon base changes for introducing a His substitution at each of seven amino-acid residues in a polypeptide (3A), given the natural coding sequence for these residues (3B), changes in the first or first two codon positions of each of the seven codons (3C) and resulting distribution of substitution residues at each position (3D);

FIGS. 4A-4C show the arrangement of variable light-chain (V_{L}) and variable heavy chain (V_{H}) CDRs in a synthetic scFv anti-TNF-α antibody gene (4A), the application of walk-through mutagenesis for introducing an aspartate amino acid at each of the 14 residues 56-69 in the V_{H} CDR2 region of the antibody (4B), and the minimum codon substitutions at eighteen different base positions needed for introducing aspartic at each of the fourteen different residue positions (4C);

FIGS. 5A-5C show the arrangement of light-chain and heavy chain CDRs in a synthetic scFv anti-TNF-α antibody gene (5A), and the amino acid sequences for three anti-TNF-α antibodies for the V_{H} (5B) and V_{L} (5C) chains;

FIGS. 6A-6D shows doping ratios of nucleotide bases for achieving a desired ratio of substituted amino acids in a walk-through mutagenesis procedure for introducing alanine (6A), leucine (6B), tyrosine (6C), and proline (6D) into each position of theCDR2 region of E2D7 V_{H} chain;

FIGS. 7A-7D show representative distributions of amino acid substitutions into the CDR2 region of E2D7 V_{H} chains using the coding sequences shown in 6A-6D, respectively;

FIGS. 8 illustrates steps in the screening of anti-TNF-α antibodies formed in accordance with the presence invention for high binding affinity based on equilibrium binding to TNF-α;

FIG. 9 shows equilibrium binding curves for antibody-expressing cells prior to selection (circles), after one round of selection (light triangles), after two rounds of selection (dark triangles), and for the D2E7 anti-TNF-α reference antibody;

FIGS. 10A and 10B show mutations in the V_{H} (10A) and V_{L} (10B) CDR regions of a scFv human anti-TNF-α antibody that are associated with enhanced equilibrium binding affinity (1.5 fold or higher for K_{D} of EC₅₀ relative to the reference antibody D2E7);

FIG. 11 illustrates steps in the screening anti-TNF-α antibodies formed in accordance with the presence invention for high binding affinity based on binding kinetic with respect to TNF-α, for determining antibody K_{off} constants;

FIGS. 12A and 12B show mutations in the V_{H} (12A) and V_{L} (12B) CDR regions of a scFv human anti-TNF-α antibody that are associated with enhanced K_{off} binding values (1.5 fold or higher for K_{off} relative to the reference antibody);

FIGS. 13A and 13B show beneficial mutations in the V_{H} (13A) and V_{L} (13B) CDR regions of a scFv human anti-TNF-α antibody, representing the combination of mutations shown in FIGS. 10A and 10B, and 12A, and 12B, for equilibrium and kinetic binding constants, respectively;

FIGS. 14A-14F show the design of degenerate oligonucleotides used in forming libraries that encode combinations of the beneficial mutations from FIGS. 13A and 13B, in all combinations of V_{H} CDR1, CDR2, and CDR3 (FIGS. 14A-14C, respectively), and all combinations of V_{L} CDR1, CDR2, and CDR3 (FIGS. 14D-14E, respectively);

FIG. 15 illustrates the oligonucleotide assembly for producing the D2E7 wild type scFv coding sequence;

FIG. 16A-16D illustrate steps in the production of an LTM V_{H} CDR2 library;

FIG. 17A-17D illustrate steps in the production of a multiple LTM V_{H} CDR library;

FIG. 18 shows an array of LTM library combinations in both V_{H} and V_{L} CDRs;

FIG. 19 shows the construction of a yeast expression vector for displaying proteins of interest on the extracellular surface of S. cerevisiae;

FIG. 20 is a FACS plot of binding of biotinylated TNFα and streptavidin FITC to D2E7 scFv;

FIG. 21 exemplifies a subset of improved clones having a lower EC₅₀ values with respect to the D2E7 antibody;

FIGS. 22A-22C are FACS plots showing a selection gate (the R1 trapezoid) for identifying only those clones that expressed the scFv fusion with a higher binding affinity to TNF-α than the D2E7 antibody (22A), the distribution of binding affinities of the total LTM library (22B), and a post sort FACS analysis (FIG. 21 right panel) to confirm that >80% of the pre-screen anti-TNF-α scFv clones were within the predetermined criteria;

FIG. 23 demonstrates the effect of two clones, 3ss-35 and 3ss-30 having a higher relative K_{off} compared to D2E7;

FIGS. 24A and 24B identify mixed mutation clones, showing 63 unique sequences for scFv anti-TNF-α clones recovered from the mixed mutation WTM libraries screened by k_{off} assays in the V_{H} and V_{L} chains, respectively.

FIGS. 25A-25g shows a Biacore determination of binding kinetics of anti-TNF-α D2E7 wild type (25A) and six affinity enhanced anti-TNF-α scFv clones (25B-25G);

FIG. 26 is a comparison of normalized dissociation rates between the different anti-TNF-α scFvs, also showing that of D2E7;

FIGS. 27A and 27B show amino acid substitutions in the identified K_{off} clones of the scCF anti-TNF-α light chain (27A) and heavy chain (27B);

FIG. 28 is a graphical analysis of L929 TNF-α dose response curve from the Table 3 results. The double headed arrow indicates the effective window range of TNF-α concentration;

FIG. 29 shows a graphical analysis of L929 dose response at 175 pg/mL TNF-α; FIG. 30: shows a graphical analysis of L929 dose response at 350 pg/mL TNF-α; and

FIG. 31 is a dose response survival curves on L929 cells in TNF-α neutralization by affinity enhanced anti-TNF-α CBM clones (A1, 2-44-2,1-3-3, 2-6-1) in comparison with the anti-TNF-α positive controls Humira and D2E7.

### DETAILED DESCRIPTION OF THE INVENTION

I. Definitions

The terms below have the following definitions herein unless indicated otherwise.

The term “human TNF-α” or “TNF-α” refers to the human cytokine that exists as a 17 kD secreted form and a 26 kD membrane associated form, the biologically active form of which is composed of a trimer of noncovalently bound 17 kD molecules., as described, for example, by Pennica, D., et al. (1984) Nature 312:724-729; Davis, J. M., et al. (1987) Biochemistry 26:1322-1326; and Jones, E. Y., et al. (1989) Nature 338:225-228.

The term “antibody”, as used herein, is intended to refer to immunoglobulin molecules comprised of four polypeptide chains, two heavy (H) chains and two light (L) chains inter-connected by disulfide bonds. Each chain consists of a variable portion, denoted V_{H} and V_{L} for variable heavy and variable light portions, respectively, and a constant region, denoted C_{H} and C_{L} for constant heavy and constant light portions, respectively. The C_{H} portion contains three domains CH1, CH2, and CH3. Each variable portion is composed of three hypervariable complementarity determining regions (CDRs) and four framework regions (FRs).

The term “antibody” also encompasses antibody fragments, such as (i) an Fab fragment, which is a monovalent fragment consisting of the V_{L}, V_{H}, C_{L} and C_{H}1 domains; (ii) a F(ab′)₂ fragment, a bivalent fragment comprising two Fab fragments linked by a disulfide bridge at the hinge region; (iii) an Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR).

Furthermore, although the two domains of the Fv fragment, V_{L} and V_{H}, are coded for by separate genes, they can be joined by recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). The term antibody also encompasses antibodies having this scFv format.

The term “human antibody”, as used herein, is intended to include antibodies having variable and constant regions derived from human germline immunoglobulin sequences.

The term “humanized antibody” is intended to include antibodies in which one or more of the regions or domains of the antibody is derived from a non-human source, e.g., an antibody in which one of the heavy- or light-chain CDRs is derived from a mouse anti-TNF-α antibody, that is, has the same coding sequence or the same amino acid sequence or a sequence more closely related to a mouse anti-TNF-α than to a human anti-TNF-α antibody.

The term “recombinant antibody”, as used herein, is intended to include all human antibodies that are prepared, expressed, created or isolated by recombinant means, such as antibodies expressed using a recombinant expression vector transfected into a host cell.

The term “isolated antibody”, as used herein, is intended to refer to an antibody that is substantially free of other antibodies having different antigenic specificities.

A “neutralizing antibody”, as used herein refers to an antibody whose binding to TNF-α results in the inhibition of the biological activity of TNF-α, as assessed by measuring one or more indicators of TNF-α, such as TNF-α-induced cellular activation or TNF-α binding to TNF-α receptors. These indicators of biological activity can be assessed by standard in vitro or in vivo assays known in the art.

The term “K_{off}”, as used herein, is intended to refer to the off rate constant for dissociation of an antibody from the antibody/antigen complex, as determined from a kinetic selection set up.

The term “K_{D}”, as used herein, refers to the dissociation constant of a particular antibody-antigen interaction, and describes the concentration of antigen required to occupy one half of all of the antibody-binding sites present in a solution of antibody molecules at equilibrium, and is equal to K_{off}/Kₒₙ, the on and off rate constants for the antibody. The association constant K_{A} of the antibody is 1/K_{D}. The measurement of K_{D} presupposes that all binding agents are in solution. In the case where the antibody is tethered to a cell wall, e.g., in a yeast expression system, the corresponding equilibrium rate constant is expressed as EC₅₀, which gives a good approximation of K_{D}.

The term “reference anti-TNF-α antibody” refers to the scFv antibody disclosed in U.S. Pat. Nos. 6,509,015 and 6,090,382. This antibody has a coding sequence derived exclusively from human germline. It is also identified herein as E2D7 scFv antibody, and by the amino acid sequence SEQ ID NO: 1.

The three-letter and one-letter amino acid abbreviations and the single-letter nucleotide base abbreviations used herein are according to established convention, as given in any standard biochemistry or molecular biology textbook.

II. Generating Enhanced-Affinity Anti-TNF-α Antibodies

This section describes methods for generating high-affinity anti-TNF-anti-TNF-α antibodies, in accordance with the invention. The general approach is to employ look-through mutagenesis (LTM) to produce a set of coding sequences that contain a selected amino acid substitution at each of the amino-acid residue positions in each of the light-chain and heavy-chain variable regions (CDRs).

Typically, the coding sequences encode an scFv anti-TNF-α antibody, and are contained in a vector used for transforming a suitable expression system such as a yeast expression system. For each of the V_{L} and V_{H} chains, the selected mutations may be placed at a selected position in one, two or all three CDRs of the variable chain. Anti-TNF-α antibodies produced by the expression system are then screened for high binding affinity, typically having a K_{D} (EC₅₀) or K_{off} that is substantially lower, typically at least 1.5 fold and preferably at least 2 fold lower than the D2E7 scFv antibody identified by SEQ ID NO:1, when measured under identical conditions. When measured according to the equilibrium (EC₅₀) or kinetic binding (K_{off}) methods described below, the high-affinity antibodies have EC₅₀ values less that about 10⁻⁸ M and/or K_{off} rate constants of less than 10⁻⁴ sec⁻¹, the highest affinities yet reported for anti-TNF-α antibodies. The LTM method preferably employs a representative subset of nine amino acids, as described below.

Once CDR mutations associated with enhanced affinity are identified, by LTM, these mutations are used to guide the construction of a library of coding sequences from which even higher-affinity antibodies can be expressed and selected. Among the libraries that may be encoded are:

(a) a combinatorial library of coding sequences that encode combinations of the V_{L} and V_{H} CDR amino-acid sequence variations identified by the LTM method;

(b) a walk-through mutagenesis library encoding, for at least one of the CDRs, the same amino acid substitution at multiple amino acid positions within that CDR; and

(c) a library of localized saturation mutation sequences encoding, for at least one of said CDRs, all 20 natural L-amino acids at an amino acid position that admits to a sequence variation identified by the LTM method.

These libraries are used to encode antibodies in a suitable expression system, such as a yeast expression system allowing identification of the desired high-affinity antibodies.

A. Look through Mutagenesis (THM)

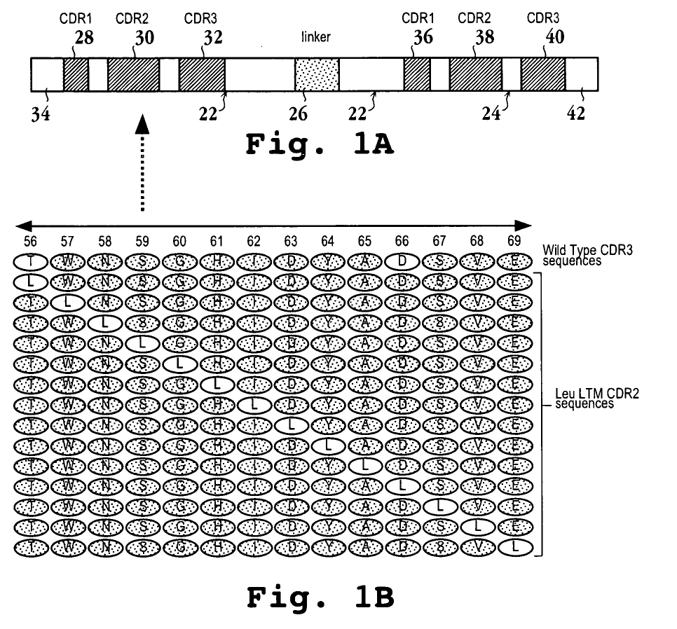

The purpose of look-through mutagenesis (LTM) is to introduce a selected substitution at each of target mutation positions in a region of a polypeptide, e.g., the CDR regions of the variable antibody chain. Unlike combinatorial methods or walk-through mutagenesis (WTM), which allow for residue substitutions at each and every position in a single polypeptide, LTM confines substitutions to a single selected position. This feature is illustrated in FIGS. 1A and 1B. As shown in FIG. 1A, the antibody, indicated at 20, is composed of a variable heavy (V_{H}) chain 22, a variable light (V_{L}) chain 24 and a peptide linker 26 joining the two chains. V_{H} chain 22 is in turn composed of three hypervariable CDR regions 28, 30, and 32 (light shading, also denoted herein as CDR1, CDR2, and CDR3, and D1, D2, D3, respectively), and four framework regions (FRs) regions, such as region 34 (dark shading). Similarly, the variable light (V_{L}) chain is composed of three hypervariable CDR regions 36, 38, and 40 (light shading, also denoted herein as CDR1, CDR2, and CDR3, and D4, D5, D6, respectively), and four framework regions (FRs) regions, such as region 42 (dark shading).

FIG. 1B shows the fourteen-residue amino acid sequence of the V_{H} CDR2 region of the wildtype CDR1 (top line) and below that, fourteen sequences having a single leu substitution at each of the positions along the CDR. The purpose of the LTM method illustrated in FIG. 1B is to substitute a single Leu residue at each of the fourteen positions 56-69. This is accomplished by generating, in addition to the wildtype coding sequence, fourteen additional coding sequences that individually provide an Leu TTG or TTA codon at each one of the fourteen different codon positions. A total of fourteen different peptides are generated, and no “undesired” or multiple-substitution sequences are produced.

B. Walk-through Mutagenesis (WTM)

The object of walk-through mutagenesis (WTM) is to investigate the effect on a polypeptide of substituting a selected amino acid, e.g., His, at each or substantially each of the amino acid positions in a selected portion of the polypeptide. In the usual case, the selected-amino acid substitutions are placed at each of a plurality of contiguous amino acid positions, where the target region for mutations is typically between 3-30 amino acid. The method is carried out so that the desired substitutions are produced with the minimum number of base substitutions in the coding sequences for target potion of the polypeptide, and the native (non-mutated) amino acid is preserved in at least coding sequence. That is, in the set of coding sequences needed to effect a single amino acid substitution at each target position, there is at least one coding sequence for the native polypeptide and at least one for each of the desired substitutions.

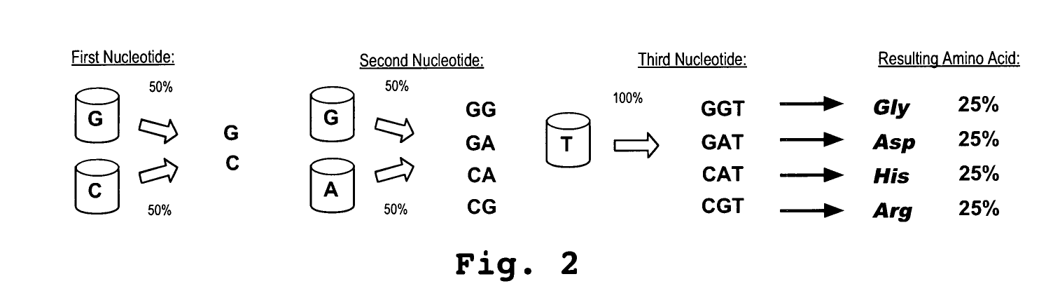

The walk-through method is illustrated in FIG. 2, which shows the base substitutions needed to produce a desired Gly to His substitution in a coding sequence containing a GGT codon for Gly. Since there are both Gly and His codons with a third-position T base (GGT and CAT, respectively), the minimum number of base substitutions needed to encode both amino acids are G and C at the first position, and G and A at the second codon position. As seen, the resulting codons include four equally likely permutations, one encoding Gly, one encoding His, and two “undesired” codons for Asp and Arg.

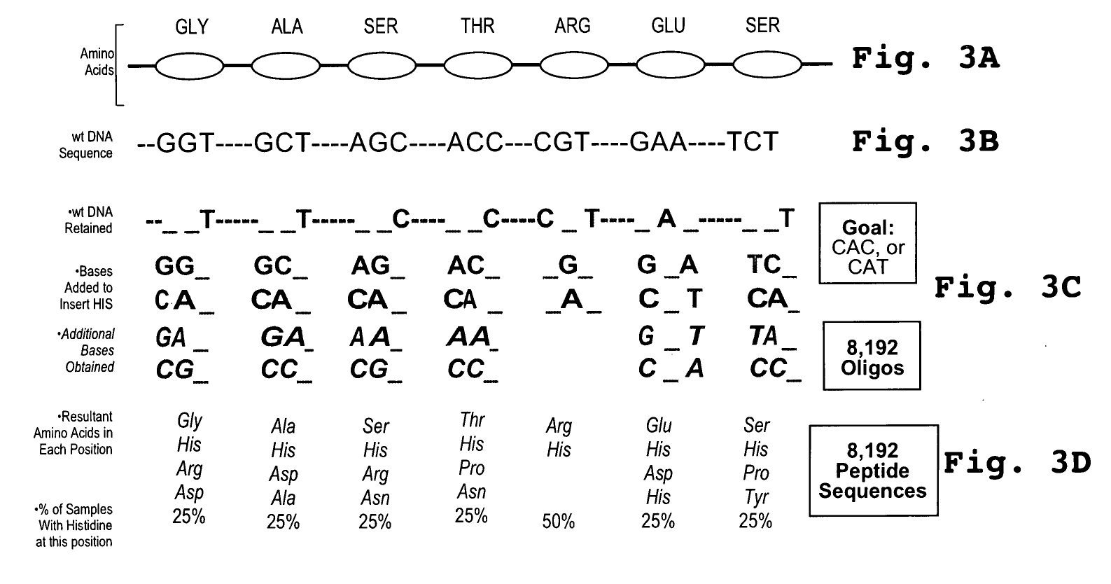

FIGS. 3A-3D illustrate the application of the same method for generating coding sequences in which a His is substituted at each position of the seven-mer amino acid sequence shown in FIG. 3A. As above, the objective is to generate a minimum set of coding sequences, at least one of which preserves the original amino acid at each position, and sequences in which His is substituted at each of the seven positions. The coding sequence for the “wildtype” seven-mer sequence is shown in FIG. 3B. As indicated at the top of FIG. 3C, the goal is to generate coding sequences that contain either a CAC or a CAT His codon at each position, and preserve the original amino-acid sequence in at least one sequence. The needed base substitutions can then be determined from a comparison of the wildtype sequence with the bases that needed for the substitution sequences. The middle frame in FIG. 3C shows the bases needed to insert both a His or the original amino acid at each position. For example, for the first codon, a 1:1 mixture of G and C at the first base position, and a 1:1 mixture of G and A at the second position produces four codons, one of which encodes Gly, one, His, and one each for “undesired” amino acids Arg and Asp. In the case of the fifth codon, for Arg, the native CGT codon can be expanded to include both Arg and His by introducing either a G or A base at the second position, as seen in FIG. 3C.

The total number of different coding sequences is 2¹³ or 8,192, and the total number of different peptide sequences is 4⁶×2 or 8,192. These numbers are to be compared with the total possible number of coding sequences produced with randomly generated coding sequences (4²¹) and the total number of different amino acid sequences that could be produced (20⁷). Accordingly, the walk-through method also produces a much higher percentage of the desired mutants (25%-50% in the examples shown in FIG. 3D) than mutations generated randomly.

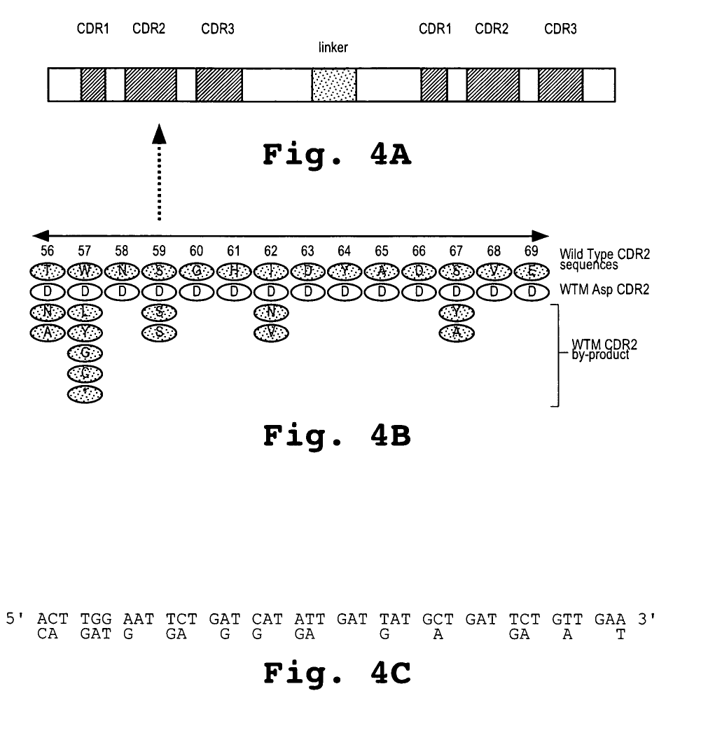

The walk-through method is illustrated in FIGS. 4A-4C, for substitution of an Asp (D) residue for each of the fourteen amino acid residues at positions 56-69 in the V_{H} CDR1 domain of the reference anti-TNF-α antibody, whose structural components are shown in FIG. 4A, similar to FIG. 1A. The wildtype coding sequence and the 18 base substitutions required to form an Asp codon at each of the 14 amino-acid residue positions are given in FIG. 4C. These eighteen substitutions yield 2¹⁸ or 262,144 different coding sequences. In FIG. 4B is shown the amino acid residues that will be introduced at each eighteen V_{H} CDR2 positions by these coding sequences, including the “undesired” substitutions at six of the positions.

The objective of WTM, as noted above, is to generate the smallest set of coding sequences that encode both the wildtype amino acid sequence, and sequences in which each residue in a selected region or regions of a polypeptide is substituted with a single selected amino acid. The amino acid selected for substitution within each CDR is preferably chosen from among those that are identified in the LTM approach above, that is, amino acids associated, in a particular CDR, with enhanced binding activity. In an exemplary embodiment, the one or more amino acids selected for substitution are those that represent beneficial mutations in more than one position of a CDR. For example, the CDR1 region of the V_{L} chain contains lysine substitutions at each of three of the 11 CDR1 positions, suggesting that this region may benefit from multiple substitutions of a positive amino acid. A suitable WTM library would then contain codons for multiple Lys, His, or Arg substitutions within this CDR. The section below discusses doping techniques for controlling the total number of the selected amino acid that are substituted into an one CDR.

C. Combinatorial Methods

In the combinatorial approach, coding sequences are generated which represent combinations of the beneficial mutations identified by LTM. These combinations may be combinations of different beneficial mutations within a single CDR, mutations within two or more CDRs within a single antibody chain, or mutations within the CDRs of different antibody chains.

One combinatorial approach resembles the WTM method except that the selected codon substitutions within the CDRs are the different beneficial amino-acid substitutions identified by LTM. Thus, not every residue position in an antibody CDR will contain a mutation, and some positions will have multiple different amino acids substituted at that position. Overall, many if not all, combinations of beneficial mutations within a CDR or an antibody chain will be represented by at least one of the coding sequences in the library. As will be seen below, this coding-sequence library can be prepared by a modification of the WTM method, except that instead placing codons for a single amino acid at each different position in the variable coding region, the codons that are introduced are those corresponding to all beneficial mutations detected in the LTM method. In order to keep the size of this library manageable, the mutations may be confined to one of the two heavy or light chains only. This combinatorial approach is detailed below.

In a second approach, individual gene fragments containing a single CDR region, and having a codon variation encoding all combinations of beneficial mutations within CDR reconstructed, e.g., by gene shuffling methods, to produce V_{L} and V_{H} chain coding sequences having combinations of beneficial mutations in all CDRs of a given chain or all CDRs in both chains.

D. Localized Saturation Mutagenesis

In this approach, the beneficial mutations identified by LTM are used to identify “active” regions of the CDRs at which different types of amino acid substitutions are shown to produce beneficial mutations. The library of coding sequences in this approach are designed to encode up to and including each of the 20 amino acids at each of the identified “hot spots” in one or more of the six CDRs of the antibody. Conversely, the approach may be carried out by identifying the “cold spots” and designing coding sequences that saturate all CDR positions except the cold-spot sites.

E. scFv Coding Libraries

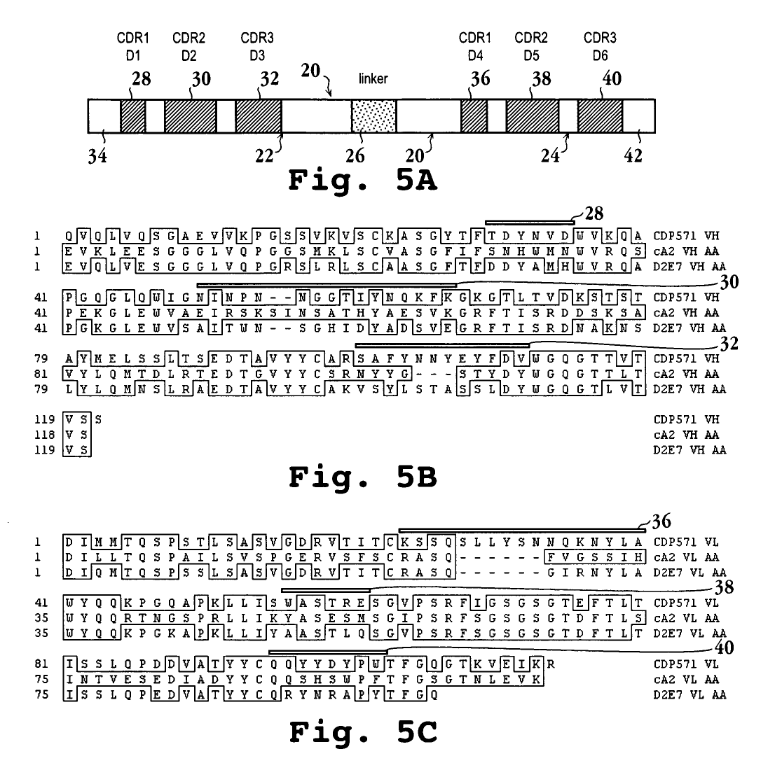

FIGS. 5A-5C illustrate the arrangement and representative sequences of a scFv anti-TNF-α antibody 20. The arrangement of antibody regions of scFV anti-TNF-α antibody is shown in FIG. 5A, and is similar to that shown in FIG. 1A and FIG. 4A. FIG. 5B gives the aligned amino acid sequences of the variable heavy chain in three anti-TNF-α antibodies, designated CDP571, cA2, and reference antibody D2E7. The CDR1, CDR2, and CDR3 regions of the chain are shown by heavy overlining at 28, 30, and 32. Thus, for example, the 5-mer CDR1 of the D2E7 variable heavy chain has the sequence DYAMH and the 12-mer CDR3 regions of the same antibody chain have the sequence DYADSVEGRFTI. Similarly, FIG. 5C gives the aligned amino acid sequences of the variable light chain in same antibodies, where the three CDRs are identified by overlining.

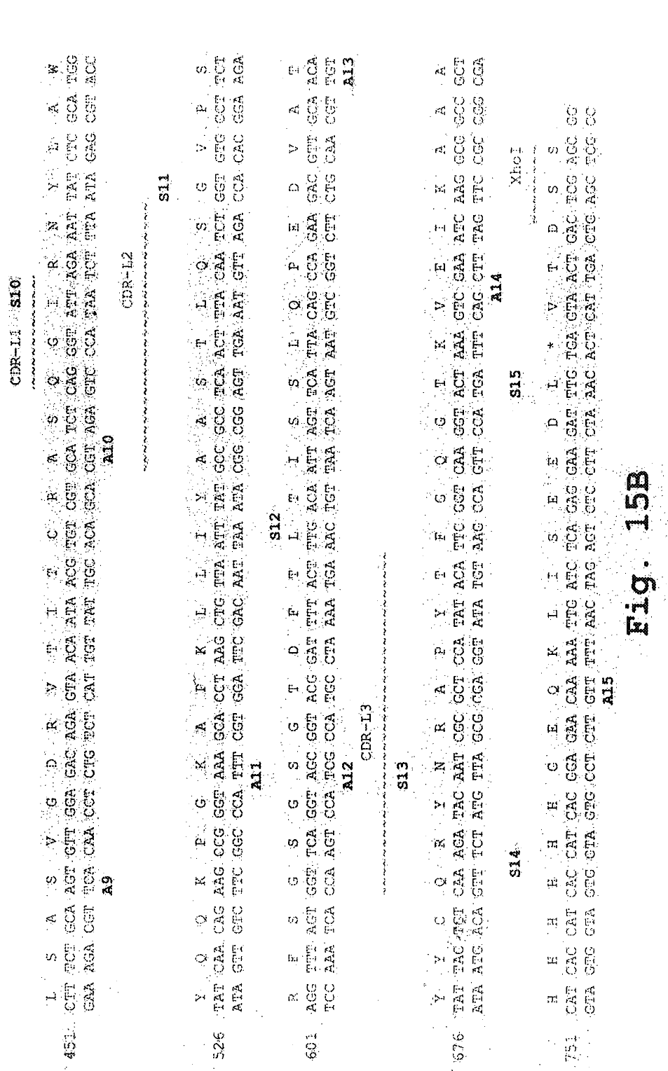

The synthesis of the coding sequence of the D2E7 scFv reference antibody having the amino-acid sequence identified by SEQ ID NO:1 is described in Example 1. Briefly, the D2E7 wild type scFv gene (approximately 1 kb) was assembled in vitro by PCR of 30 oligonucleotides shown in FIG. 15, each oligonucleotides a portion of the contiguous full length D2E7 scFv sequence. There were 15 sense and 15 anti-sense oligonucleotides that were on average, 40 base pairs in length (ranging in size from 35 to 70) and overlapped complementary regions of approximately 20 base pairs on the neighboring upstream and downstream oligonucleotides. The 30 nucleotides are identified herein as SEQ ID NOS: 52-81.

As will be seen below, the LTM and WTM methods is applied to the coding and amino acid sequences of one or more of the D2E7 V_{H} or V_{L} chain CDR regions, for purposes of generating antibodies whose binding constant is substantially enhanced with respect to the reference scFv E2D7 antibody. More specifically, the LTM and WTM techniques described above are used to create pools of oligonucleotides with mutations in one or more CDRs of the light or heavy chain of the reference antibody. These oligonucleotides are synthesized to include some of the surrounding framework. These pools of oligonucleotides are utilized to generate all possible V_{L} and V_{H} chains in which there are mutations in single, double, and triple CDRs (CDR1, 2, and 3) using single overlap extension PCR (SOE-PCR). Methods for generating pools of LTM CDR oligonucleotides, and WTM oligonucleotides are detailed in Example 2. Methods for generating LTM and WTM libraries from these pools are detailed in Example 3.

For example, to create the pool of V_{H} chains in which both V_{H} CDR1 and V_{H} CDR2 are mutated and V_{H} CDR3 is wild-type, the CDR1 oligonucleotides are first used as templates and SOE-PCR is conducted to link the CDR2 oligonucleotides to generate the doubly mutated pool. Considering that each CDR may be either wild-type or mutant, there are eight possible combinations for each of the pools of V_{L} and V_{H} chains.

Combining the eight V_{L} and eight V_{H} pools creates 64 V_{L}-V_{H} combinations (scFvs), one of which is wild-type, and 63 of which are non wild-type. Each of the 64 V_{L}-V_{H} combinations (including the wild-type sequence) is termed a subset of the whole LTM™ or WTM™ scFv library. An LTM™ or WTM™ scFv library is generated for each amino acid selected for substitution. The number of amino acid sequences represented within each subset library depends on the length of the CDR, the amino acid sequence within the CDR, and the LTM™ or WTM™ oligonucleotide design strategy.

The individual scFv libraries are constructed using the splice overlap extension polymerase chain reaction (SOE-PCR) method (Horton, et al., 1989), providing a fast and simple method for combining DNA fragments that do not require restriction sites, restriction endonucleases, or DNA ligase. In SOE-PCR two oligonucleotides are first amplified by PCR using primers designed so that the PCR products share a complementary sequence at one end. Under PCR conditions the complementary sequences hybridize, forming an overlap. The complementary sequences then act as primers, allowing extension by DNA polymerase to produce a recombinant molecule. These methods are detailed in Example 3.

There are two additional constraints imposed on the WTM and LTM procedures discussed above. The first concerns the total number of amino acids whose substitution into the CDR regions of the antibody is examined. Rather than examine the effect of all 20 natural L-amino acids, it is more efficient to employ a subset of these that represent the chemical diversity of the entire group. One representative subset of L-amino acids that meets this criterion includes the alanine, aspartate, lysine, leucine, proline, glutamine, serine, tyrosine, and histidine. These amino acids display adequate chemical diversity in size, charge, hydrophobicity, and hydrogen bonding ability to provide meaningful initial information on the chemical functionality needed to improve antibody properties. The choice of a subset of amino acids may also be based on the frequency of certain amino acids in CDRs. For example, given a choice between tyrosine and phenylalanine to represent an amino acid with an aromatic side chain, tyrosine might be a better choice of its significantly higher preponderance in antibody binding sites.

Implicit in the selection of a representative subset of amino acids is that a beneficial mutation, that is, one that enhances binding activity or neutralizing activity of the antibody, produced by substitution of an amino acid in the representative subset will reasonably predict that the one or more amino acids that are related to the specific mutation in size, charge, hydrophobicity and/or hydrogen binding ability will also produce the same positive effect on antibody activity. In the present case, each of the nine representative subset amino acids will be taken to include the related amino acids given in parenthesis: Ala (Gly); Asp (Glu); Lys (Arg); Leu (Ile and Val); Pro; Gln (Asn); Ser (Thr); Tyr (Phe Trp); and His. Thus, a positive mutation from say, Asp to Tyr, will predict a similar effect by a Gly to Phe or Gly to Trp, and a positive mutation from, say Met to Ser, will predict a positive mutation from Met to Thr.

A second constraint imposed on coding sequences for WTM (but not LTM) involves the use of doping to control the percentage of sequences that code for either the wild-type or the mutation, with 12% to 50% of the sequences having the mutation. Doping the bases allows one to fine-tune the number of amino acid substitutions in the CDR of a WTM™ library member. In the above example for lysine substitutions, it is unlikely that it would be advantageous for a CDR to have lysine in all seven positions, or even in the majority of positions simultaneously. Utilizing doping, oligonucleotides are synthesized that maintain an average of 2-4 lysine substitutions per molecule or per CDR.

In the case of mixed-mutation WTM, doping can additionally be used to equalize the expected distribution of mutations at any given position. For example, if one base produces an expected level of a given substitution of 25%, and another, an expected level of a different amino acid of only 12.5%, the relative amounts of the two bases may be in a 1:2 ratio, to equalize the probabilities of seeing both mutations in equal amounts.

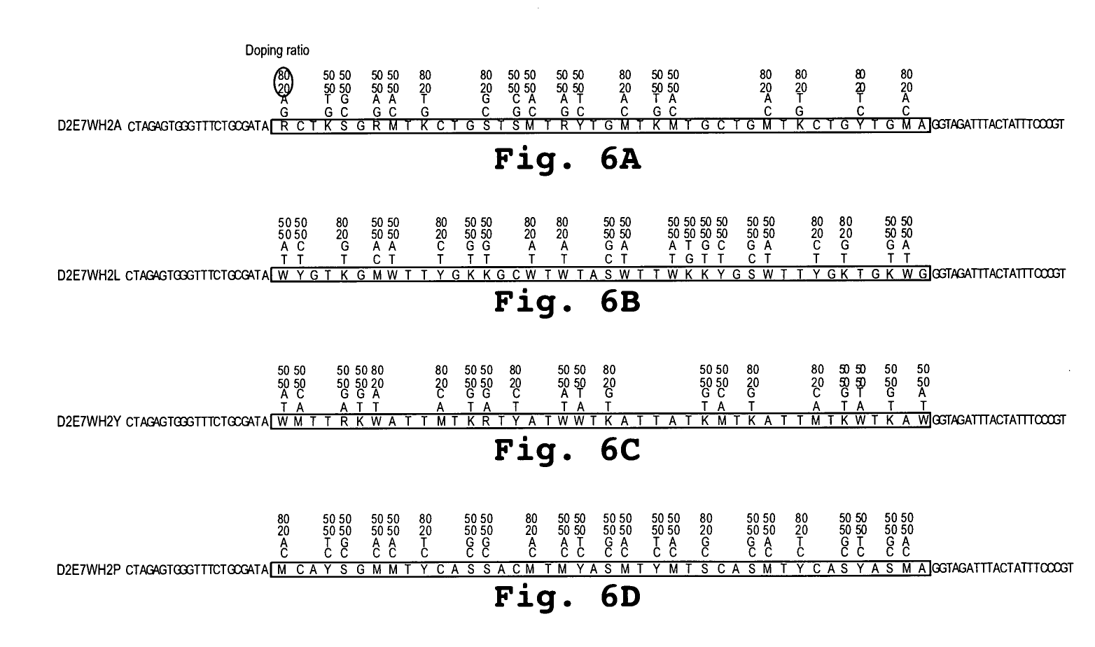

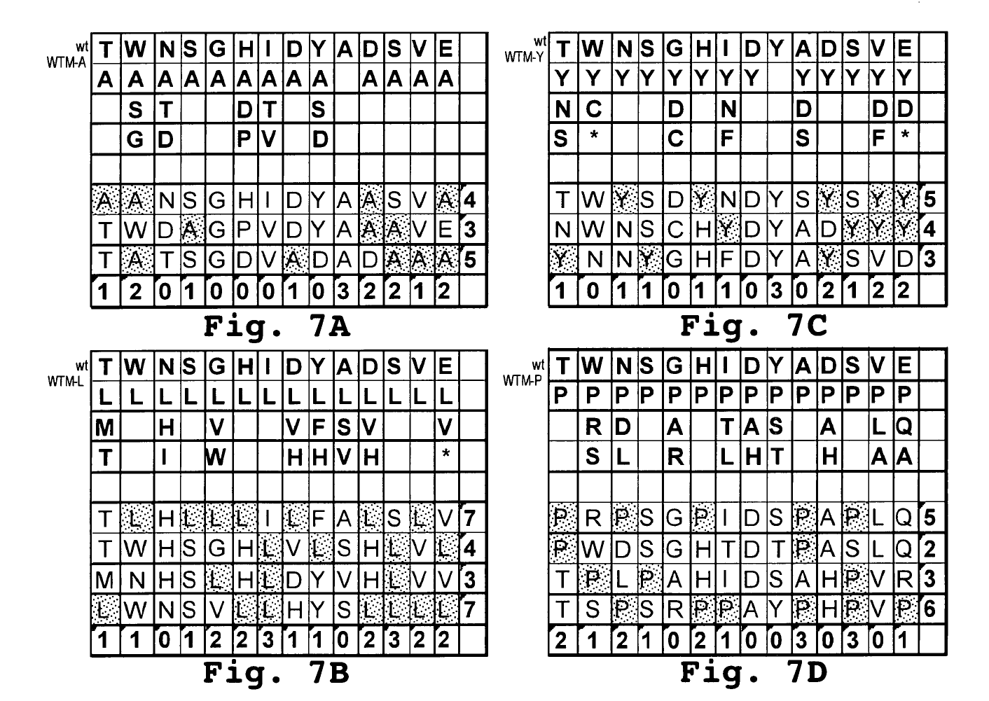

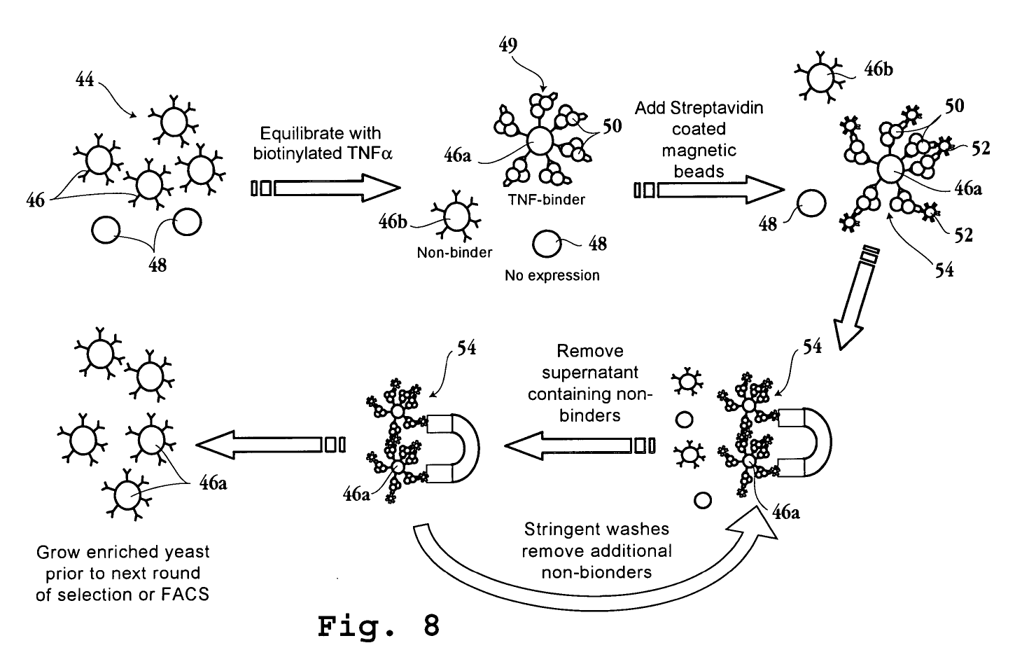

FIGS. 6A-6D show WTM codon substitutions for introducing either alanine (FIG. 6A), leucine (FIG. 6B), tyrosine (FIG. 6C), or proline (FIG. 6D) at one or more of the 14 residue position in D2E7 V_{H} CDR2 region of the reference antibody defined by the sequence TWNSGHIDYADSVE. In each figure, the sequence letters indicated either a nucleotide (A, C, G, or T) or a two-nucleotide mix, as indicated by the two nucleotides indicated over the letter. Thus, for example, in the first few two-nucleotide mixes shown at the left in FIG. 6A, R is a mixture of A and g, K a mixture of T and G, S and mixture of G and C, and so on.

The relative molar amounts of each nucleotide in a two-nucleotide mix is indicated in the figures, and is typically either 4:1 (80:20) or 1:1 (50:50). The 4:1 ratios are “doping” ratios used to achieve an average of 3-4 mutations of the selected amino acid (for FIG. 6A, Ala) per expressed antibody. Thus, the 4:1 mixture of Ag at the first substituted coding position would predict a Thr to Ala substitution in only 1 out of every five antibody chains expressed. Representative distributions of amino acid substitutions produced by the four coding sequence libraries from FIGS. 6A-6D are given in FIGS. 7A-7D, respectively. Each figure shows the (D2E7) wt sequence, the WTM positions at which an Ala (FIG. 7A), Leu (FIG. 7B), Tyr (FIG. 7C), and Pro (FIG. 7D) can occur, and also additional “undesired” amino acids encoded by various of the oligo coding sequences. The lower portion of each figure shows actual representative sequences produced, including the number of the desired amino acid substitutions in the entire region. As seen, the number of substitutions varies from 2 to seven in each of the representative sequences.

The design of oligonucleotide WTM and LTM libraries is preferably carried out using software coupled with automated custom-built DNA synthesizers. Implementation of the LTM™ and WTM™ strategies involves the following steps. After selection of target amino acids to be incorporated into the CDRs, the software determines the codon sequence needed to introduce the targeted amino acids at the selected positions within the CDRs. Optimal codon usage is selected for expression in the selected display and screening host, e.g., the yeast expression system (see below). The software also eliminates any duplication of the wild-type sequence that may be generated by this design process. It then analyzes for potential stop codons, hairpins, loops and other problematic sequences that are then fixed. The software determines the ratios of bases added to each step in the synthesis (for WTM™) to fine tune the amino acid incorporation ratio. The completed LTM™ or WTM™ design plan is then sent to the DNA synthesizer, which performs automated synthesis.

F. Yeast Cell Expression and Surface Display

A variety of methods for selectable antibody expression and display are available. These include bacteriophage, Escherichia coli, and yeast. Other methods of antibody expression may include cell free systems such as ribosome display and array technologies which allow for the linking of the polynucleotide (i.e., a genotype) to a polypeptide (i.e., a phenotype) e.g., Profusion™ (see, e.g., U.S. Pat. Nos. 6,348,315; 6,261,804; 6,258,558; and 6,214,553). Convenient E. coli expression system, have been described by Pluckthun and Skerra. (Pluckthun, A. and Skerra, A., Meth. Enzymol. 178: 476-515 (1989); Skerra, A. et al., Biotechnology 9: 273-278 (1991)). By attaching a signal sequence, such as the ompA, phoA or pelB signal sequence to either the 5′ or 3′ end of the antibody coding sequence, the antibodies can be expressed for secretion into the periplasmic space of E. coli (Lei, S. P. et al., J. Bacteriol. 169: 4379 (1987)).

While each of these has been utilized for antibody improvement, the yeast display system affords several advantages (Boder and Wittrup 1997). Yeast can readily accommodate library sizes up to 10⁷, with 10³-10⁵ copies of each antibody being displayed on each cell surface. Yeast cells are easily screened and separated using flow cytometry and fluorescence-activated cell sorting (FACS) or magnetic beads. Yeast also affords rapid selection and regrowth. The eukaryotic secretion system and glycosylation pathways of yeast allow for a much larger subset of scFv molecules to be correctly folded and displayed on the cell surface than prokaryotic display systems.

The yeast display system utilizes the a-agglutinin yeast adhesion receptor to display proteins on the cell surface. The proteins of interest, in this case, scFv WTM™ and LTM™ libraries, are expressed as fusion partners with the Aga2 protein. These fusion proteins are secreted from the cell and become disulfide linked to the Aga1 protein, which is attached to the yeast cell wall (see Invitrogen, pYD1 Yeast Display product literature). In addition, there are carboxyl terminal tags included which can be utilized to monitor expression levels and/or normalize binding affinity measurements. Methods for selecting expressed antibodies having substantially higher affinities for human TNF-α, relative to the reference D2E7 antibody, will now be described. Details of the yeast expression system and its use in antibody display are given in Example 4.

III. Selecting and Expressing Enhanced-Affinity Antibodies

This section describes methods for selecting enhanced affinity antibodies using either an equilibrium binding analysis method to measure K_{D} (or EC₅₀) or a kinetic binding analysis to determine a K_{off} constant. Several high-affinity antibodies produced by both binding criteria are disclosed. The two groups of enhanced-binding antibodies have many mutations in common and some that are unique to each method of affinity determination. The groups, when combined, provide a map of beneficial mutations in the V_{H} and V_{L} CDRs of the antibody that are associated with enhanced binding activity.

A Anti-TNF-α Antibodies with Enhanced EC₅₀.

The antibodies disclosed in this section have EC₅₀ values which are at least 1.5 and up to 2-5 fold lower than the measured EC₅₀ for the reference D2E7 antibody, when both antibodies are expressed in scFv form, and measured under identical equilibrium binding conditions.

FIG. 8 illustrates the protocol for determining EC₅₀ based on binding equilibrium. The method employs a biotinylated TNF-α antigen and streptavidin coated magnetic beads to select high affinity molecules from yeast libraries, according to published procedures (Yeung and Wittrup, 2002 and Feldhaus et al., 2003). In the present case, hTNF-α is biotinylated according to standard procedures (see Example 4C), with biotinylated TNF-α being indicated at 50 in the figure. Yeast cells transformed with the scFv coding libraries, shown at 44 in the figure, will contain a mixture of cells expressing anti-TNF-α antibodies, such as cells 46, and cells non-expressing cells, such as indicated at 48. The objective of the screening procedure is to identify those high-affinity expressing cells, such as cell 46a, from low-affinity expressing cells, indicated at 46b.

Initially, the yeast cells are equilibrated with biotinylated TNF-α, producing a mixture of cells having bound biotinylated TNF-α, indicated at 49, and low-affinity and non expressing cells. Following equilibration binding to TNF-α, streptavidin coated beads, such as beads 52, are added to the mixture, forming a binding complex 54 consisting of high-affinity expressing cells, biotinylated TNF-α, and magnetic beads. The complexes are isolated from the mixture using a magnet 56, and the bound complex is washed several times under stringent conditions to remove complexes of low-affinity cells and non-specifically bound cells. The resulting purified complexes are released from the complexes, by treatment with a suitable dissociation medium, to yield cells enriched for expression of high-affinity antibodies. In one exemplary screening method, the isolated cells are plated at low density, and clonal colonies are then suspended in medium at a known cell density. The cells are then titrated with biotinylated TNF-α by addition of known amounts of TNF-α, as indicated, e.g, from 10 pM to 1000 nM. After equilibration, the cells are pelleted by centrifugation and washed one or more times to remove unbound TNF-α, then finally resuspended in a medium containing fluoresceinated streptavidin. The fluoresceinated cells are scanned FACS to determine an average extent of bound fluorescein per cell. This method is described in Examples 5 and 6.

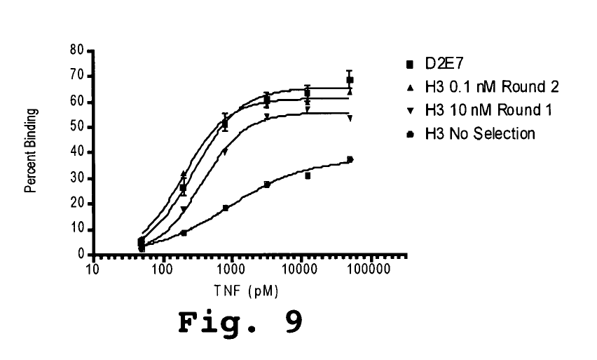

FIG. 9 shows TNF-α binding curves for cells before selection (circles), after 1 round of selection (light triangles), after 2 rounds of selection (dark triangles) and for cells expressing D2E7 (squares). As seen, the EC₅₀ value of the expressed antibody decreased from about 10 nM after one round of screening to about 0.1 nM after two rounds of screening, e.g., about the same EC₅₀ as measured for the reference antibody.

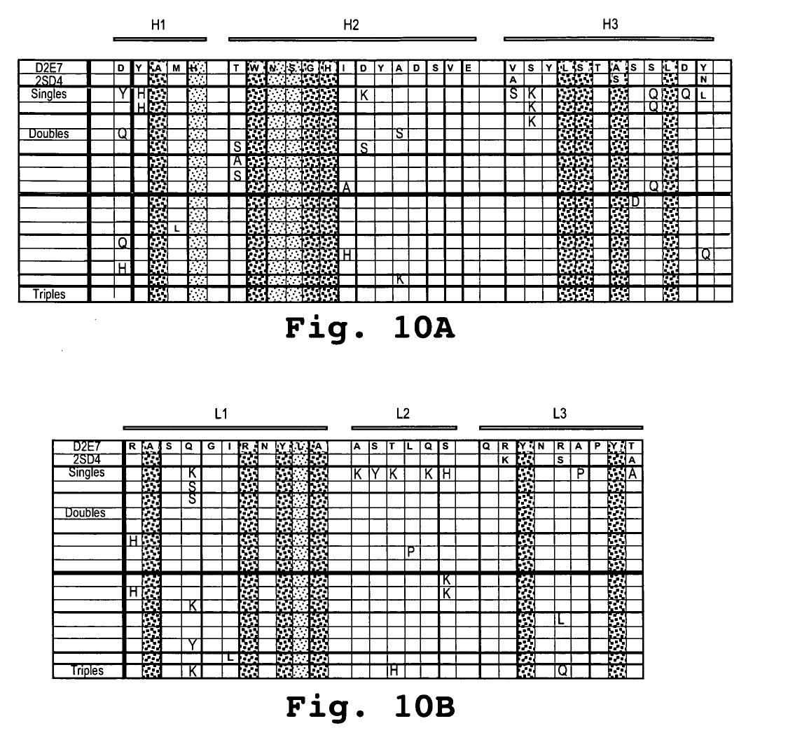

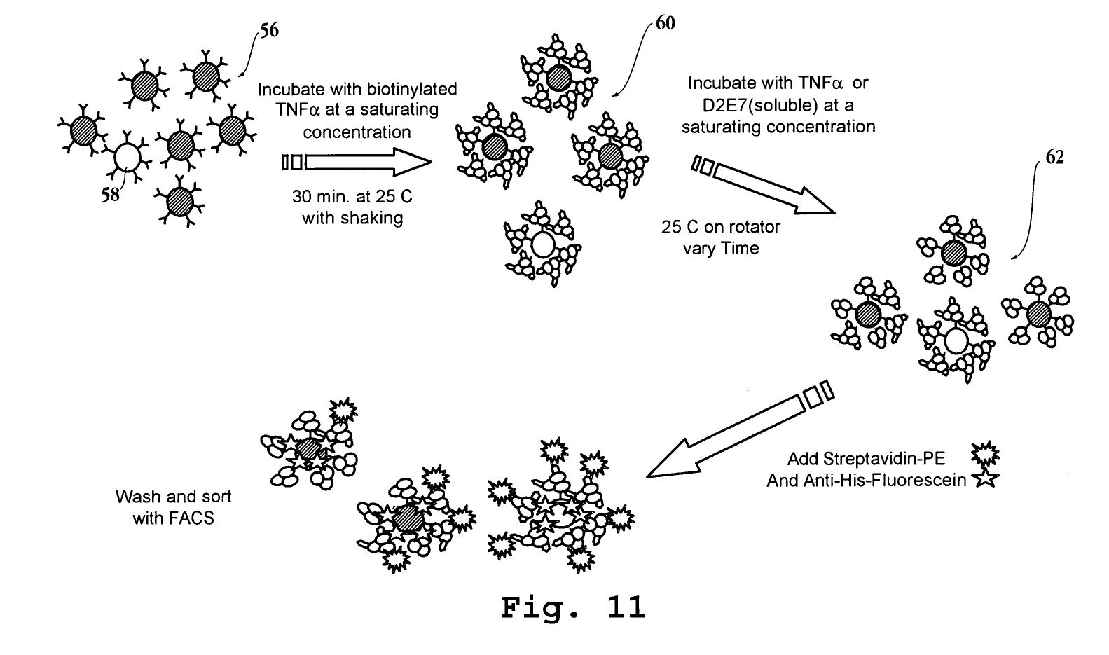

In the initial LTM study, LTM coding libraries for both the V_{H} and V_{L} chains were constructed, with the other chain containing a wildtype (D2E7) amino acid sequence. Each coding sequence in a V_{H} or V_{L} library contained a single mutation for a selected representative amino acid in one, two, or all three CDRs in that chain. The library sequences were used, as above, in constructing scFv coding sequences, and the scFv sequence used to transform the above yeast expression system, and antibodies having binding affinities, measured as EC₅₀, of less than 0.05 nM (less than half the EC₅₀ of D2E7) were selected and sequenced in the CDR regions. The individual amino acid mutations associated with the enhanced-affinity scFv antibodies are shown in FIGS. 11A and 10B for V_{H} and V_{L} CDR regions, respectively. The figures represent a total of 30 sequences, include mutations in each CDR, single-, double-, and triple-CDR mutations, and include each of the nine different amino acids tested. Each CDR also includes one position in which no mutations was found, e.g., the Ala position of V_{H} CDR1 and the W, G, and H, positions of the V_{H} CDRR2 region.

Collectively, the mutations shown in FIGS. 10A and 10B can be represented in a heavy- or light-chain sequence containing the wildtype amino acid sequence of D2E7, and at each CDR position that allows a mutation, the wildtype residue and each of the one or more selected mutations. Thus, for example, the V_{H} CDR1 region corresponding to residues 31-35 is represented as Xₐₐ₃₁ Xₐₐ₃₂ A Xₐₐ₃₄ H, where Xₐₐ₃₁=D, Y, Q, or H; Xₐₐ₃₂=Y or H, and Xₐₐ₃₄=M or L, where three CDRs in either the V_{L} or V_{H} chain include at least at least one of the indicated CDR mutations with respect to the D2E7 sequence, and may include multiple, e.g., 2-5 or more of the specified mutations.

It will be understood that a substitution mutation in the identified antibody sequences may represent the amino acid shown or its equivalent-class amino acid, as discussed above. Thus, in the above example, Xₐₐ₃₄=M or L will also cover, in one embodiment, the sequence Xₐₐ₃₄=M or L or I or V. Once high-affinity cells have been selected, the binding affinities of individual molecules displayed on the surface of clonal yeast cells is determined, as above. This allows for rapid identification of molecules with improved affinity.

B Anti-TNF-α Antibodies with Enhanced K_{off}.

The antibodies disclosed in this section have K_{off} values which are at least 1.5 and up to 2-5 fold lower than the measured measured K_{off} for the reference D2E7 antibody, when both antibodies are expressed in scFv form, and measured under identical kinetic binding conditions. The antibodies were generated using the LTM libraries above for each of the V_{L} and V_{H} chains, where the antibodies were expressed, as above, in scFv format.

FIG. 11 illustrates the kinetic binding setup used in measuring k_{off} for mutated anti-TNF-α antibodies. The method employs a biotinylated TNF-α antigen and a fluoresceinated strepavidin to those high affinity molecules having a low k_{off} constant, according to published procedures (refs). The figure shows yeast expression cells, such as cells 56, which includes a population of cells having displayed antibodies with different k_{off} values, the lowest values (highest affinity) antibodies being associated with cell 58 having the lightest shading in the figure. The cells are incubated with a saturating amount of biotinylated hTNF-α under conditions, e.g., 30 minutes at 25° C., with shaking, to effectively saturate displayed antibodies with bound antigen, indicated at 60 in the figure.

The cells are then incubated with either non-biotinylated TNF-α, or with a competitive soluble antibody, e.g., D2E7, both at saturating conditions, for a selected time sufficient to reduce the percentage of biotinylated TNF-α bound to the cells, in both cases, as a function of the off rate of the antigen. Following incubation, the cells are centrifuged, and washed to remove unbound biotinylated TNF-α and/or soluble competitive antibody, yielding cells 62, each of which contains a ratio of biotinylated and native TNF-α in proportion of the antibody's K_{off}.

Details of the method are given in Example 7.

The k_{off} values are then determined by incubating the cells with a fluoresceinated streptavidin (streptavidin-PE) and a fluoresceinted cell market (anti-his-fluorescein), washing the cells, and sorting with FACS. The k_{off} value is determined from the ratio of the two fluorescent markers, according to known methods.

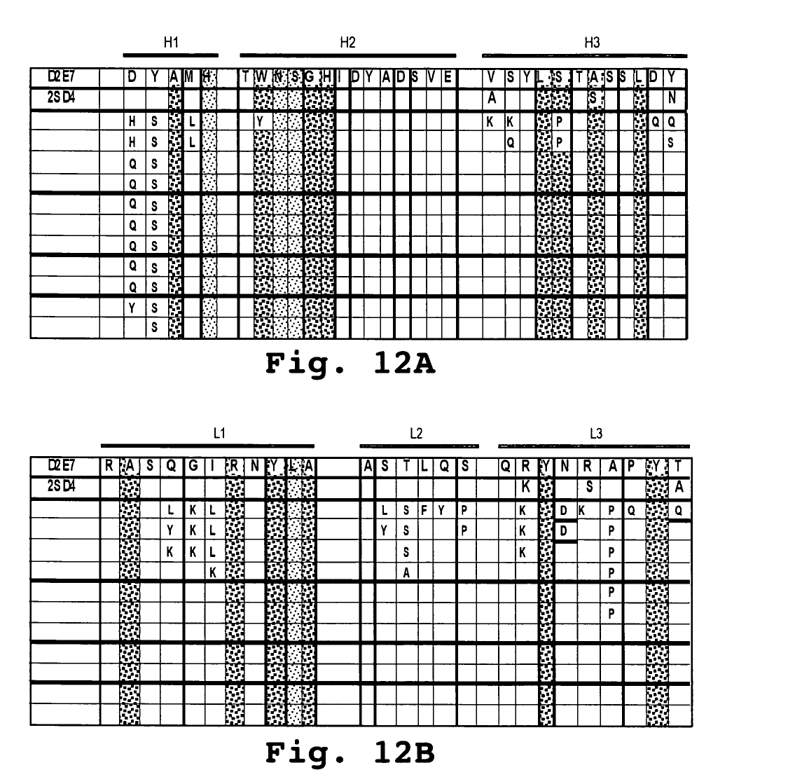

FIGS. 12A and 12B show 26 unique sequences for scFv antiTNF-α antibodies selected in accordance with the above method, using LTM coding sequences containing single mutations at one, two or all three CDRs in either the V_{H} chain (FIG. 12A) or V_{L} chain (FIG. 12B), as described in Section IIIA above. As above, the mutations can be represented in a heavy- or light-chain sequence containing the wildtype amino acid sequence of D2E7, and at each CDR position for which a beneficial mutation was identified, the wildtype residue and each of the one or more beneficial mutations. Thus, for example, the V_{H} CDR1 region corresponding to residues 31-35 is represented as Xₐₐ₃₁ Xₐₐ₃₂ A Xₐₐ₃₄ H, where Xₐₐ₃₁=D, Y, Q, or H; Xₐₐ₃₂=S, and Xₐₐ₃₄=L, where the combined light and heavy chain sequences include at least at least one of the indicated CDR mutations with respect to the D2E7 sequence, and may include multiple, e.g., 2-5 or more of the specified mutations. As above, it is understood that a substitution mutation in the identified antibody sequences may represent the amino acid shown or its equivalent-class amino acid.

C. Production of Soluble Antibodies

Antibodies from high-affinity clones from above are sequenced to identify high-affinity mutations. Antibodies of interest are subcloned into a soluble expression system, such as Pichia pastoris or E. coli, and soluble antibody, e.g., scFv antibody, is produced. A number of commercially available vectors and cell lines for soluble antibody expression, including those from Invitrogen (i.e. pPIC9) are available. These systems are routinely used to generate soluble single chain or full-length antibody. Expression of high-affinity antibodies in accordance with the present invention has yielded greater than 1 mg per liter soluble scFv in the P. pastoris expression system (Invitrogen). Purification of proteins is facilitated by the presence of a His-tag at the C-terminus of the molecule, in the case of single chains or by protein A or protein G columns for full-length antibodies. Soluble single chain and full-length antibodies will be generated to obtain BIAcore affinity measurements and for use in the assays described below.

IV. Libraries of Antibody Coding Sequences

As noted above, beneficial mutations (yielding a substantially higher K_{D} or k_{off}) identified as above by LTM may be used to generate libraries of coding sequences useful for selecting combinations of mutations capable of producing additive beneficial binding effects. Ideally, the antibodies selected contain multiple mutations in at least one CDR, either the same or different amino acids, and/or amino acid substitutions in two or more CDRs or either the corresponding V_{H} or V_{L} antibody chain.

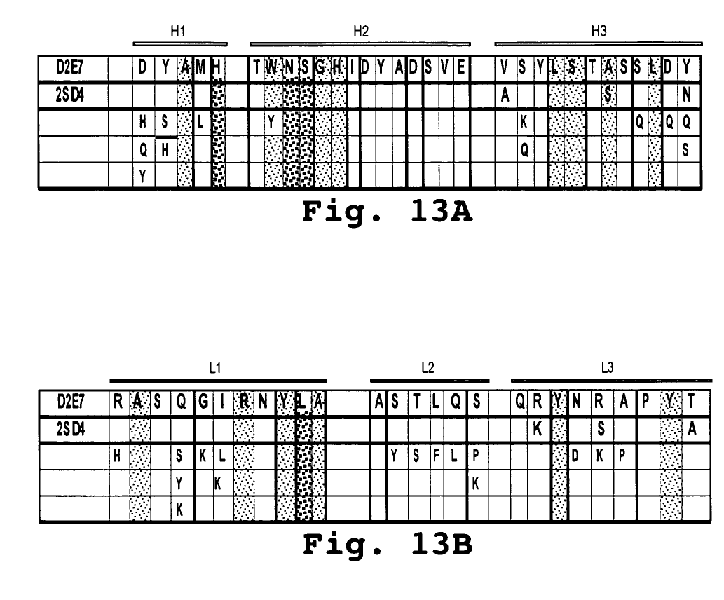

In one combinatorial approach, the beneficial mutations identified from both the equilibrium and kinetic binding selections were combined into one or both of the V_{H} and V_{L} chain sequences shown in FIGS. 13A and 13B, respectively. The sequence shown in FIG. 13B is associated herein with SEQ ID NO 2 which includes (i) the four constant or framework regions of D2E7 shown in FIGS. 5C, and each of the three CDR regions shown in FIG. 13B, where, the V_{H} CDR1, CDR2, and CDR3 regions are identified by SEQ ID NOS: 3, 4, and 5, respectively. Similarly, the sequence shown in FIG. 13A is associated herein as SEQ ID NO 7, which includes (i) the four constant or framework regions of D2E7 shown in FIGS. 5B, and each of the three CDR regions shown in FIG. 13A, where, the V_{H} CDR1, CDR2, and CDR3, regions are identified by SEQ ID NOS: 8, 9, and 10.

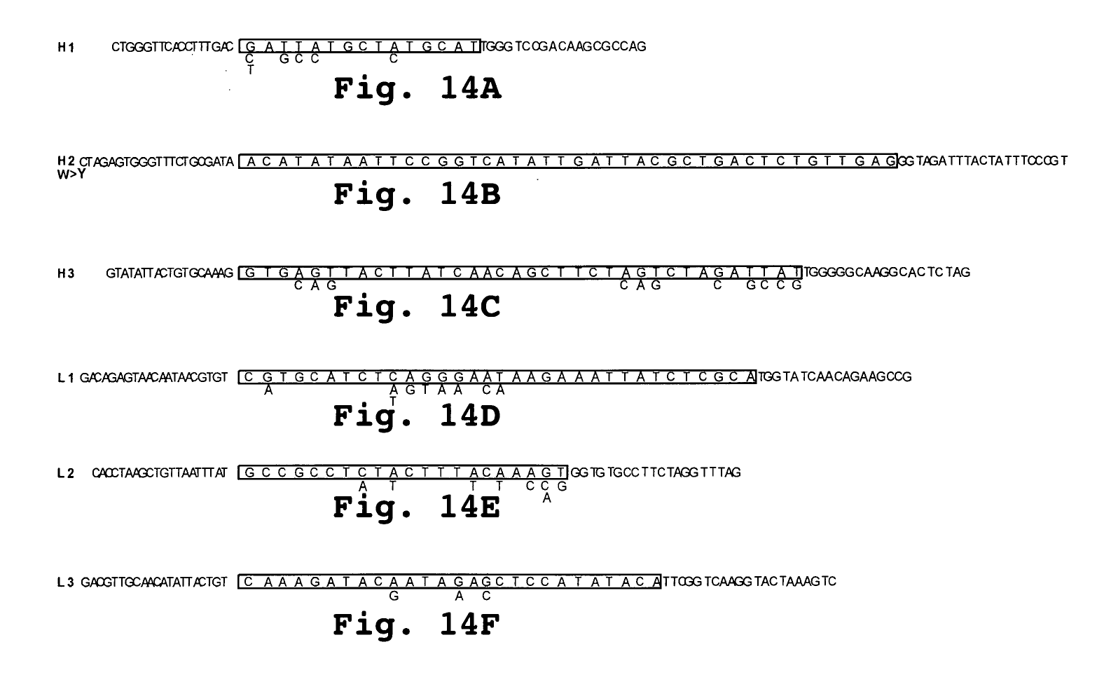

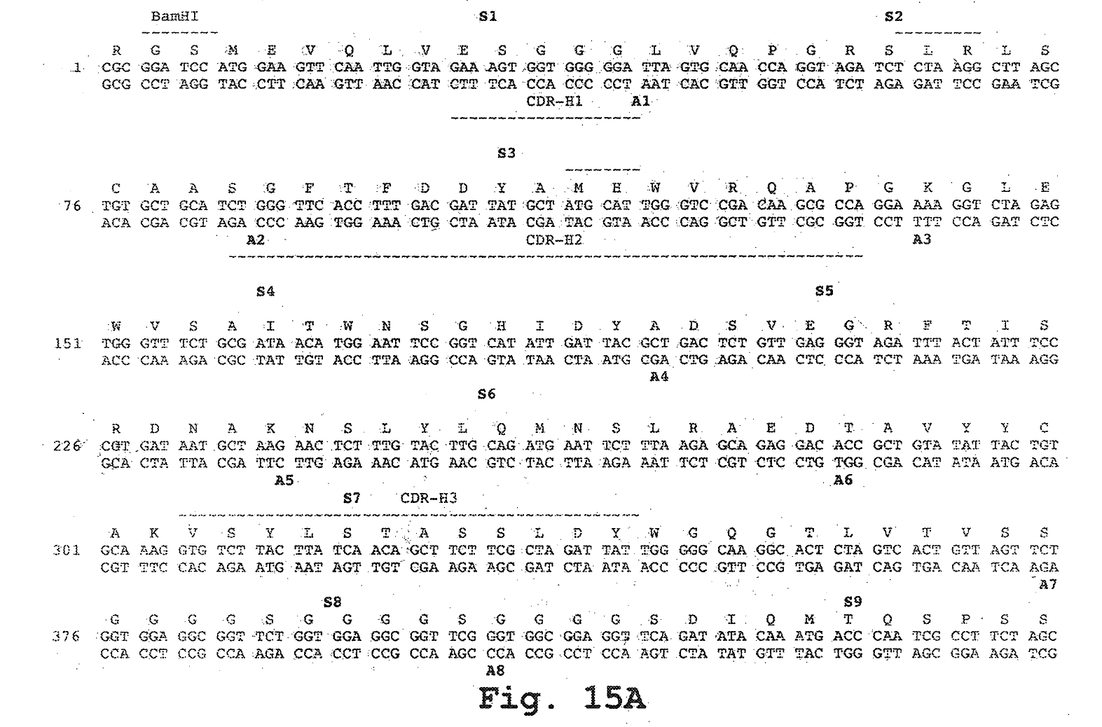

The above combinatorial libraries encoding each of the above V_{H} chain CDR1, CDR2, and CDR3 regions are shown in FIGS. 14A through 14C, and are identified herein as SEQ ID NOS: 14-16 respectively. The actual sequences identified by the sequence numbers include only the CDR-encompass sequences, and include alternative bases at the indicated position. Thus, for example, the V_{H} CDR1 coding sequence identified by SEQ ID NO 1 represents the sequence X₁AX₃X₄X₅TGCTX₁₀TGCAT, where X₁=G, C, or T, X₃=T or G, X₄=T or C, X₅=A or C, and X₁₀=A or C. Similarly, the combinatorial coding regions for the V_{L} CDR1, CDR2, and CDR3 regions are shown in FIGS. 14D-14F, respectively, and identified herein as SEQ ID NOS: 11-13.

The combinatorial CDR coding regions above are incorporated into V_{H} or V_{L} coding regions, employing framework coding regions for the corresponding constant of framework coding regions on either side of each CDR coding region, according to methods described above for construction of the LTM libraries.

These V_{H} and V_{L} combinatorial WTM libraries are then combined with wildtype (D2E&) V_{L} or V_{H} coding regions, respectively to form a library of mutated V_{H} or mutated V_{L} antibody genes, e.g., genes expressing the scFv antibody format.

The libraries are used to transfer a suitable surface display system, e.g., yeast cells, and cells are then screened, by equilibrium or kinetic selection setups to identify cells expressing antibodies with enhanced binding K_{D} or k_{off}) antibodies. As indicated above, these antibodies will contain beneficial mutations in one or more of the CDR of either the V_{L} or V_{H} chain, may contain multiple mutation in any one CDR, and the mutations may include more than one type of amino acid. Once high-activity V_{L} or V_{H} chains are identified, the method may be further extended to select for mutations occurring simultaneously in both V_{L} and V_{H} chains, by generating more limited mixed-mutation WTM libraries covering both chain CDRs.

A combinatorial library of mutations may also be generated by known gene shuffling methods, such as detailed in U.S. patent application 2003/005439A1, and U.S. Pat. No.6,368,861, and (Stemmer WP (1994) Proc Natl Acad Sci 91(22):10747-51), all of which are incorporated herein by reference. The method involves limited DNase I digestion of the collected mixed mutation clones to produce a set of random gene fragments of various pre-determined sizes (e.g. 50-250 base pairs). The fragments are then first denatured and the various separate fragments are then allowed to re-associate based on homologous complementary regions. In this manner, the re-natured fragments may incorporate differing mixed mutation CDRs in the re-assembled segments which are then extended by SOE-PCR as above, and a re-assembled chimera may then incorporate, at a minimum, at least two sets of beneficial CDR mixed mutations from each parental DNA source donor. Other mix and match techniques for generating coding sequences from CDR oligonucleotide fragments may also be used.

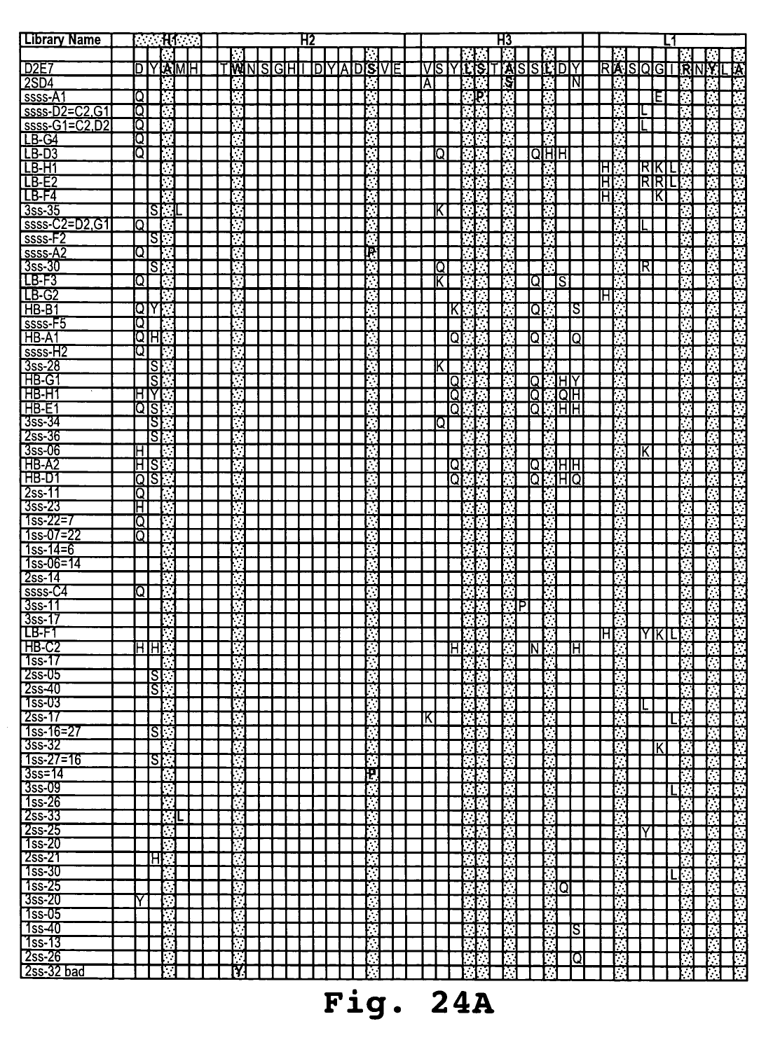

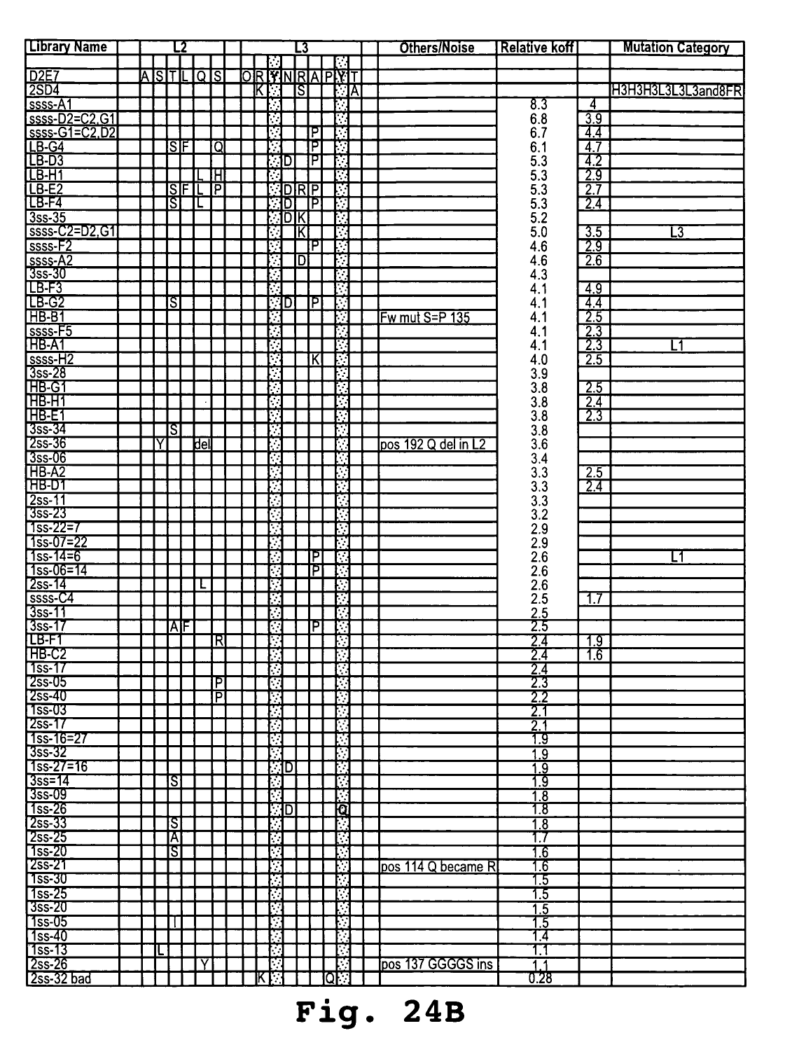

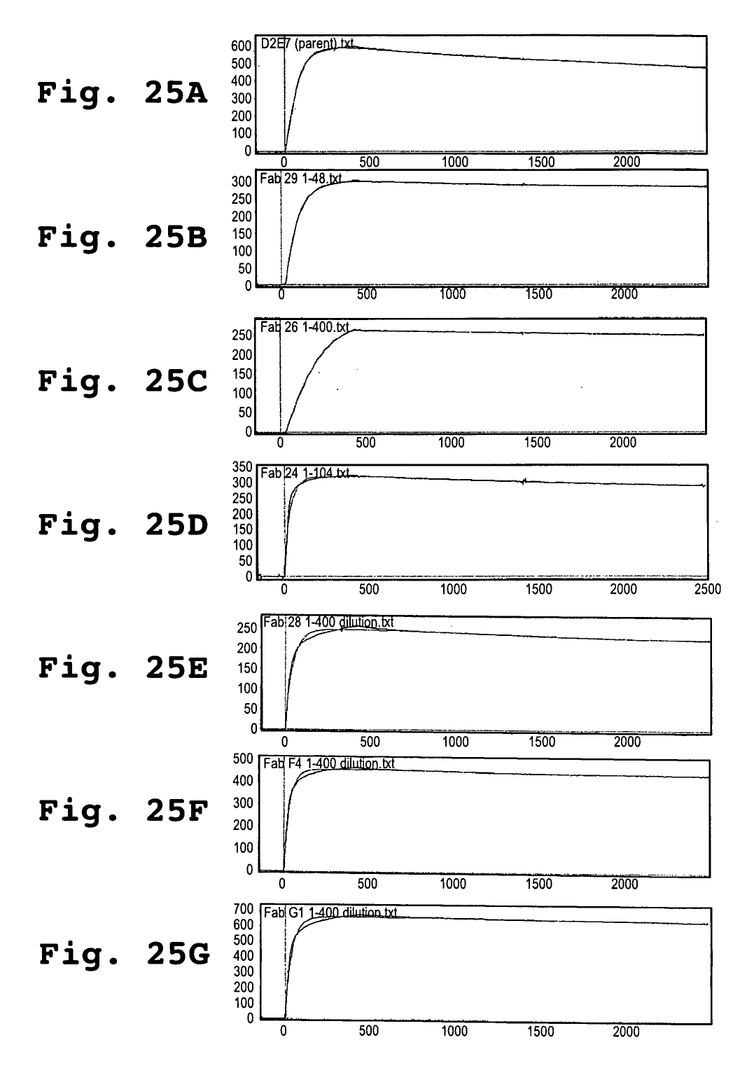

Libraries of antibody coding sequences for a WTM may be constructed as above, employing a single selected amino acid substitution within each of the CDRs, and preferably also using doping to achieve an average amino substitution of 2-4 mutations in each CDR as described above. The amino acid that is selected for each CDR is preferably one corresponding to a beneficial amino acid substitution in at least two residues of that CDR, or having similar properties as beneficial mutations that occur in two or more residues. For example, looking at FIG. 24, it is apparent that many of the beneficial mutations are polar (ionizable) amino acids, e.g., glutamine, lysine, asparagine, histidine, serine, and tyrosine, so any of these amino acids or another selected polar amino acid may be selected for WTM in the CDR-HE domain. Similarly, the CDR-L1 domain contains multiple positively charged beneficial mutations, such as lysine, histidine, and arginine, so any of these amino acids may be used for WTM in the L1-CDR domain.

Finally, the library of coding sequence constructed using the LTM beneficial mutations as a guide mutations can be a saturation sequence in which one or selected CDR positions, and preferably “hot spots”, are substituted for each of the up to and including 20 standard amino acids. These “hot spots” may be residue positions at which one or more substitutions appear in a large number of high-affinity mutants, such as the first and second CDR-H1, or the second, third, ninth, eleventh, and twelfth positions or at which several different beneficial mutations are found, such as or positions 4 and 5 of CDR-L1, positions 3, 5, and 6 or CDR-L2, position 5 of CDR-L3, position 1 of CDR-H1, and positions 2, 3, 11 and 12 of CDR-H3. The coding sequences are prepared, as above, by introducing codons for each amino acid at the one or selected beneficial mutation positions.

### EXAMPLE 1

### D2E7 V_{H} and V_{L} scFv Oligonucleotide Synthesis

A. Construction of D2E7 Wild Type scFv Gene:

The D2E7 wild type scFv gene (approximately 1 kb) was assembled in vitro by PCR of 30 oligonucleotides (FIG. 15 ) each representing a portion of the contiguous full length D2E7 scFv sequence. Synthetic oligonucleotides were synthesized on the 3900 Oligosynthesizer by Syngen Inc. (San Carlos, Calif.) as per manufacturer directions and primer quality verified by PAGE electrophoresis prior to PCR use. There were 15 sense and 15 anti-sense oligonucleotides that were on average, 40 base pairs in length (ranging in size from 35 to 70) and overlapped complementary regions of approximately 20 base pairs on the neighboring upstream and downstream oligonucleotides. The 30 nucleotides are listed in SEQ ID NO: 17.

The 30 primers were all incubated together as a mixture (5 μl of 10 uM oligonucleotide mix) and PCR assembled using 0.5 μl Pfx DNA polymerase (2.5 U/μl), 5 μl Pfx buffer (Invitrogen), 1 μl 10 mM dNTP, 1 μl 50 mM MgSO4 and 37.5 μl dH20 at 94 C for 2 min, followed by 24 cycles of 30 sec at 94 C, 30 sec at 50 C, and 1 min at 68 C and then incubated at 68 C for 5 min. The PCR assembly reaction permitted oligonucleotide overlap annealing, base-pair gap filling, and ligation of separate oligonucleotides on each strand of the DNA duplex to form a continuous full length D2E7 scFv gene. An aliquot (1 μl) of the above PCR assembly reaction was taken out for further D2E7 scFv full length amplification using an added pair of D2E7 5′ and 3′ end specific oligonucleotide primers (SEQ ID NO: 18 and 19) 2 μl each of 10 uM stock, 0.5 μl Pfx DNA polymerase (2.5 U/μl), 5 μl Pfx buffer, 1 μl 10 mM dNTP, 1 μl 50 mM MgSO4 and 37.5 μl dH20 at 94 C for 2 min, followed by 24 cycles of 30 sec at 94 C, 30 sec at 50 C, and 1 min at 68 C and then incubated for a 68 C for 5 min. The D2E7 scFv DNA from the PCR reaction was then extracted and purified (Qiagen PCR purification Kit) for subsequent Bam HI and Not I restriction endonuclease digestion as per manufacturer's directions (New England Biolabs). Full length D2E7 scFv was then subcloned into pYD1 vector and sequenced to verify that there were no mutations, deletions or insertions introduced (SEQ ID NO:1 and 6). Once verified, full length V_{H} and V_{L} D2E7 served as the wild type template for the subsequent strategies of building LTM and WTM libraries.

### EXAMPLE 2

### LTM and WTM Oligonucleotide Synthesis

In the following examples, the predetermined amino acids of CDR-H2 segment (positions 56 to 69; TWNSGHIDYADSVE) from the D2E7 wild type V_{H} section LDWVSAI-TWNSGHIDYADSVE-GRFTISR, was selected for both LTM and WTM analysis. The polypeptide sequences LDWVSAI and GRFTISR are portions of the V_{H} frameworks 2 and 3 respectively flanking CDR-H2. In the design and synthesis of V_{H} and V_{L} CDR LTM and WTM oligonucleotides, flanking framework sequence lengths were approximately 21 base pairs for SOE-PCR complementary overlap. A reference oligonucleotide coding for the above CDR-H2 wild type sequence (in bold) (SEQ ID NO: 23) containing the flanking V_{H}2 and V_{H}3 portions (lowercase letters below) is below: 5′-gta gag tgg gtt tct gcg ata-ACT TGG AAT TCT GGT CAT ATT GAT TAT GCT GAT TCT GTT GAA-ggt aga ttt act att tcc cgt-3′.

A. Design of CDR LOOK THROUGH MUTAGENESIS (LTM) Oligonucleotides

Look Through Mutagenesis analysis introduces a predetermined amino acid into every position (unless the wildtype amino acid is the same as the LTM amino acid) within a defined region. In this V_{H} CDR-H2 example, leucine LTM of V_{H} CDR-H2 involves serially substituting only one leucine at a time, in every CDR-H2 position. FIG. 1 illustrates LTM application for introducing a leucine amino acid into each of the fourteen residues (positions 56-69) in the V_{H} CDR-H2 region of D2E7 scFv. In performing leucine LTM, fourteen separate oligonucleotides encoding all possible V_{H} CDR-H2 leucine positional variants were synthesized (SEQ ID NOS:24-36) with each having only one leucine replacement codon (in bold) bordered by D2E7 wild type sequence.

CDR-H2 LTM oligonucleotides for the other eight “subset” amino acids; alanine, aspartate, lysine, leucine, proline, glycine, serine, tyrosine, and histidine were designed and synthesized in analogous manner. For example, the first aspartate (codon in bold) LTM oligonucleotide (out of the fourteen for CDR H2) replacement was (SEQ ID 38): 5′-gtagagtgggtttctgcgata-GAC TGG AAT TCT GGT CAT ATT GAT TAT GCT GAT TCT GTT GAA-ggtagatttactatttcccgt-3′.

An example of oligonucleotides for CDR H1 leucine LTM is listed in SEQ ID NOS. 41-45. As in the CDR H2 design above, 17 base pairs of wild type D2E7 framework 1 and 2 sequences (lowercase lettering) flank the CDR H1 to allow SOE-PCR assembly into the remainder of the scFv construct.

B. Design of CDR WALK THROUGH MUTAGENESIS (WTM) Oligonucleotides

To perform a Walk Through Mutagenesis (WTM), a selected amino acid is multiply substituted in different positions and in various combinations with the wild type sequence of a predetermined region. FIGS. 6A, 6B, 6C, and 6D describe the WTM oligonucleotide sequences for V_{H} CDR H2 in introducing the amino acids, alanine, leucine, tyrosine and proline respectively. FIGS. 4A-4C illustrate multiply substituting aspartate throughout the CDR-H2 using the following synthesized WTM oligonucleotide sequence: 5′-gtagagtgggtttctgcgata-RMT KRK RAT KMT GRT SAT RWT GAT KAT GMT GAT KMT GWT GAW-ggtagatttactatttcccgt-3′. (SEQ ID NO:39). Standard nucleotide nomenclature: K=G or T, M=A or C, R=A or G, S=C or G, W=A or T, Y═C or T, and N=A, C, G, or T. The degenerate oligonucleotide produced 262,144 possible different nucleotide sequence combinations which resulted in 27,648 possible amino acid sequences in CDR H2. The additional diversity introduced into CDR H2 by the degenerate oligonucleotide codons are also shown in FIG. 4B.

### EXAMPLE 3

### LTM and WTM scFv Libraries

The LTM and WTM oligonucleotides described above were then used to create pools of mutations in a single CDR of the light or heavy chain. As shown, these LTM and WTM oligonucleotides are synthesized to include approximately 20 bases of flanking framework sequences to facilitate in overlap and hybridization during PCR.

A. Introduction of Oligonucleotides and Construction of LTM Libraries.

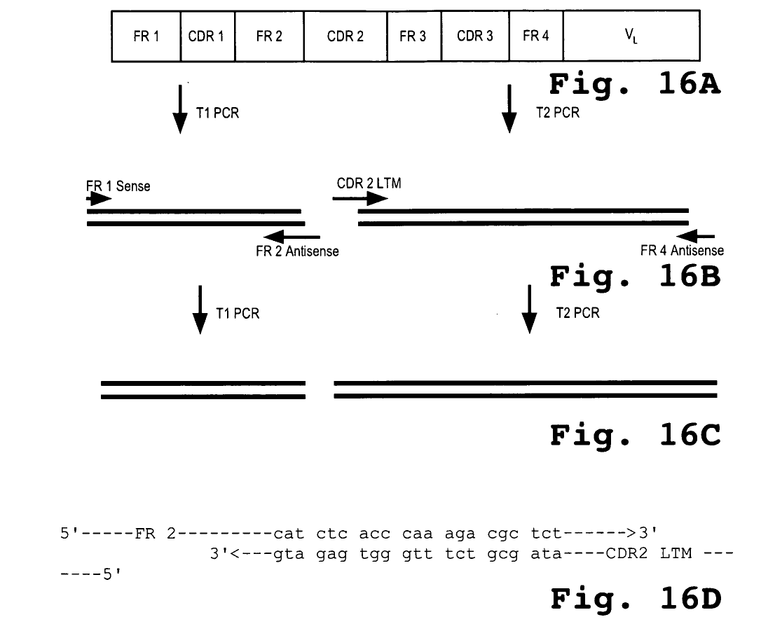

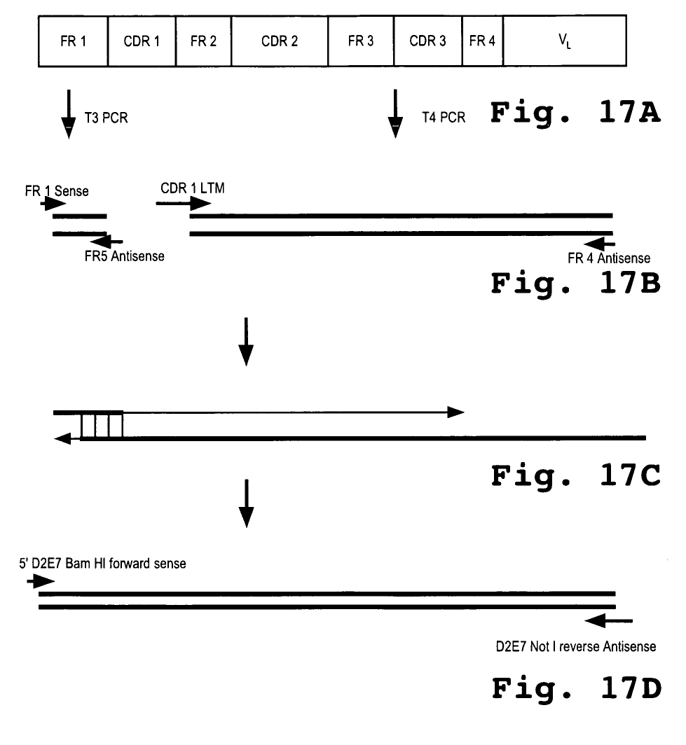

The approach in making the LTM CDR-H2 library is summarized in FIGS. 16A-16D. Separate PCR reactions, T1 and T2, were carried out using primer pairs FR1 sense (SEQ ID NO: 21) and FR2 antisense (SEQ ID NO: 22) and the above pooled CDR-2 LTM leucine oligonucleotides (for example SEQ ID NO: 24) with FR4 anti-sense primer, respectively. Primer FR1 sense contains sequences from the 5′terminus of the D2E7 gene and FR2 anti-sense contains the antisense sequence from the 3′terminus of D2E7 framework 2 so that the D2E7 CDR-H1, framework regions 1 and 2 was amplified in the T1 PCR reaction (FIGS. 16B and 16C). The primer FR4 AS contains anti-sense sequence from the 3′terminus of the D2E7 gene, CDR-2 LTM oligonucleotides contain sequences from the 5′ terminus of the D2E7 CDR2 region with the incorporated CDR-H2 LTM codon mutations to amplify the remaining portion of D2E7 (fragment CDR2, FR3, CDR3, FR4 and V_{L}) while concurrently incorporating the mutagenic codon(s). T1 and T2 PCR reactions used; 5 μl of 10 uM oligonucleotide mix, 0.5 μl Pfx DNA polymerase (2.5 U/μl), 5 μl Pfx buffer (Invitrogen), 1 μl 10 mM dNTP, 1 μl 50 mM MgSO4 and 37.5 μl dH20 at 94 C for 2 min, followed by 24 cycles of 30 sec at 94 C, 30 sec at 50 C, and 1 min at 68 C and then incubated for a 68 C for 5 min. The reactions were performed using a programmable thermocycler (MJ Research).

T1 and T2 PCR reactions were then gel purified (as per instructions in Qiagen Gel purification kit) and equimolar aliquots from both were then combined for single overlap extension PCR (SOE-PCR). SOE-PCR is a fast and simple method for combining DNA fragments that does not require restriction sites, restriction endonucleases, or DNA ligase. The T1 and T2 PCR products were designed share end overlapping complementary sequences (FIG. 16D) that would hybridize and allow PCR extension to produce a full length LTM D2E7 scFv gene. The scFv PCR extension reaction used T1 and T2 aliquots (approximately 2 ul each) with 0.5 μl Pfx DNA polymerase (2.5 U/μl), 5 μl Pfx buffer (Invitrogen), 1 μl 10 mM dNTP, 1 μl 50 mM MgSO4 and 37.5 μl dH20 at 94 C for 2 min, followed by 20 cycles of 30 sec at 94 C, 30 sec at 50 C, and 1 min at 68 C and then incubated for a 68 C for 5 min.

A set of D2E7 end specific 5′ Bam HI sense (SEQ ID NO: 18) and D2E7 3′ Not I antisense primers (SEQ ID NO: 19) was added to facilitate LTM D2E7 amplification and incorporate the restriction enzyme sites in the PCR amplicons (FIG. 16a step E). Directly added to above PCR extension reaction was 4 μl of 10 uM oligonucleotide stock, 0.5 μl Pfx DNA polymerase (2.5 U/μl), 5 μl Pfx buffer (Invitrogen), 1 μl 10 mM dNTP, 1 μl 50 mM MgSO4 and 37.5 μl dH20 at 94 C for 2 min, followed by 24 cycles of 30 sec at 94 C, 30 sec at 50 C, and 1 min at 68 C and then incubated for a 68 C for 5 min.

B. PCR Product Cloning into Yeast Cell Expression Vector DYD1:

The plasmid pYD1, prepared from an E. coli host by plasmid purification (Qiagen), was digested with the restriction enzymes, Bam HI and Not I, terminally dephosphorylated with calf intestinal alkaline phosphatase. Ligation of the pYD1 vector and the above SOE-PCR products (also digested by BamHI and NotI), E. coli (DH50) transformation and selection on LB-ampicillin (50 mg/ml) plates were performed using standard molecular biology protocols.

C. Multiple LTM CDR Libraries.

Double and Triple CDR mutations (in different combinations of CDR1, 2, and 3) are created as above but instead of using the wild type D2E7 gene as PCR template, a previously generated LTM D2E7 library is chosen instead. For example, to create V_{H} chains in which both CDR-H1 and CDR-H2 are mutated and CDR-H3 and V_{L} are wild-type, the LTM CDR-H2 mutant genes were used as templates and then SOE-PCR was conducted to incorporate the CDR-H1 oligonucleotides to generate the Double LTM mutations and summarized in FIG. 16b.

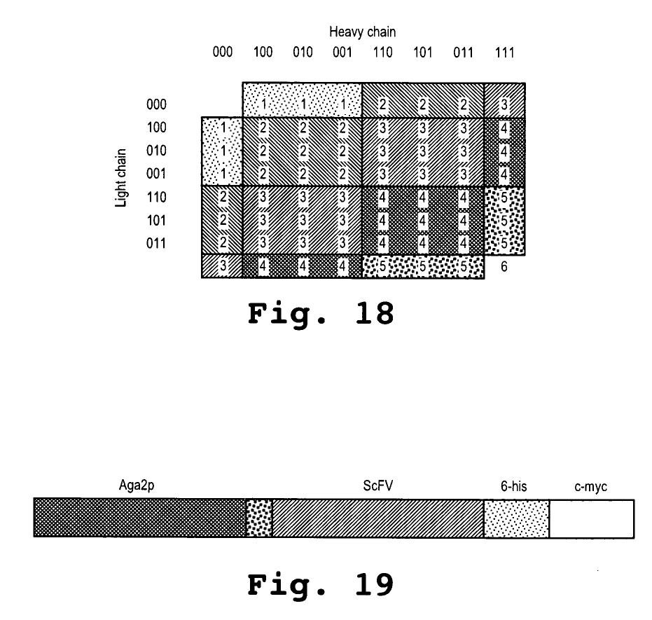

In this case, the two separate PCR reactions, T3 used primer pairs FR1 sense (SEQ ID NO: 21) and FR5 antisense (SEQ ID NO: 20) to amplify the framework region 1 (FR 1). The T4 PCR reaction utilized the pooled CDR-H1 LTM oligonucleotides (SEQ ID NO: 27) with FR4 anti-sense primer ((SEQ ID NO 24) to amplify the remaining FR 2, CDR2 LTM, FR3, CDR3, FR4 and V_{L} portions of D2E7 (FIGS. 17B). T3 and T4 PCR reactions were then purified and equimolar aliquots from both were then combined for SOE-PCR (FIG. 17C) to produce D2E7 scFv double LTM CDR-H1 and CDR-H2 library. A set of D2E7 end specific 5′ Bam HI sense (SEQ ID NO: 18) and D2E7 3′ Not I antisense primers (SEQ ID NO: 19) was added to facilitate LTM D2E7 amplification (FIG. 17D) and cloning into pYPD1 expression vector.

The double LTM CDR-H1, CDR-H2 library were then used as templates to incorporate LTM CDR-H3 oligonucleotides to make the Triple CDR H3 LTM libraries. By progressively utilizing the starting single and double LTM libraries, an more complex array of LTM library combinations in both the V_{H} and V_{L} CDR was developed (FIG. 18). For example, once the LTM CDR-H1, CDR-H2, CDR-H3 library was constructed, designated as the 111 library template in the top row of FIG. 17, introduction of LTM CDR-L1 into the 111 templates produced a library of 4 LTM CDRs (indicated by the arrow in FIG. 18).

### EXAMPLE 4

### Yeast Cell Expression System

pYD1 (FIG. 19) is an expression vector designed to display proteins of interest on the extracellular surface of Saccharomyces cerevisiae. By the sub-cloning the scFv gene into pYD1, scFvs becomes a fusion proteins with the AGA2 agglutinin receptor allowing cell surface secretion and display.

A. Transformation of Yeast Host Cells with PYD1 AGA2-scFv Constructs:

Competent yeast host cells (500 μl) was prepared as per instructions by Zymo Research Frozen-EZ yeast Kit (Catalogue #). Briefly, 500 μl of competent cells was mixed with 10-15 μg pYPD1 scFv library DNA after which 5 ml of EZ3 solution was added. The cell mixture was incubated for 45 minutes at 30° C. with occasional mixing (three times). The transformed cells were centrifuged and resuspended in Glucose select liquid media,

B. Induction of AGA2-scFv:

After grown in Glucose select media (see Invitrogen manual for composition) at 30° C. under shaking aeration conditions for 48 hours until the OD₆₀₀=7 (OD₆₀₀=1 represents 10⁷ cells/ml). The cells were then collected, re-pelleted and re-suspended in the induction medium, Galactose select media (see Invitrogen manual for composition), to an OD₆₀₀=0.9 at 20° C. for 48 hours. Expression of the Aga2-scFv fusion protein from pYD1 is tightly regulated by the GAL1 promoter and depends on galactose in the medium for promoter induction.

C. Biotinylated TNF-α Preparation:

Biotinylation of the TNF antigen can be accomplished by a variety of methods however; over-biotinylation is not desirable as it may block the epitope—antibody interaction site. The protocol used was adapted from Molecular Probes FluoReporter Biotin-XX Labeling Kit (cat# F-2610). Briefly, TNFα 300 μl of 1 mg/ml stock (Peprotech), was added to 30 μl 1M Sodium Bicarbonate Buffer at pH 8.3 and 5.8 μl of Biotin-XX solution (20 mg/ml Biotin-XX solution in DMSO). The mixture was incubated for 1 hour at 25° C. The solution was transferred to a micron centrifuge filter tube, centrifuged and washed repeatedly (four times) with PBS solution. The biotinylated-TNFα solution was collected and the protein concentration determined by OD 280.

D. FACS Monitoring of AGA2-scFv Expression and TNF□ Binding:

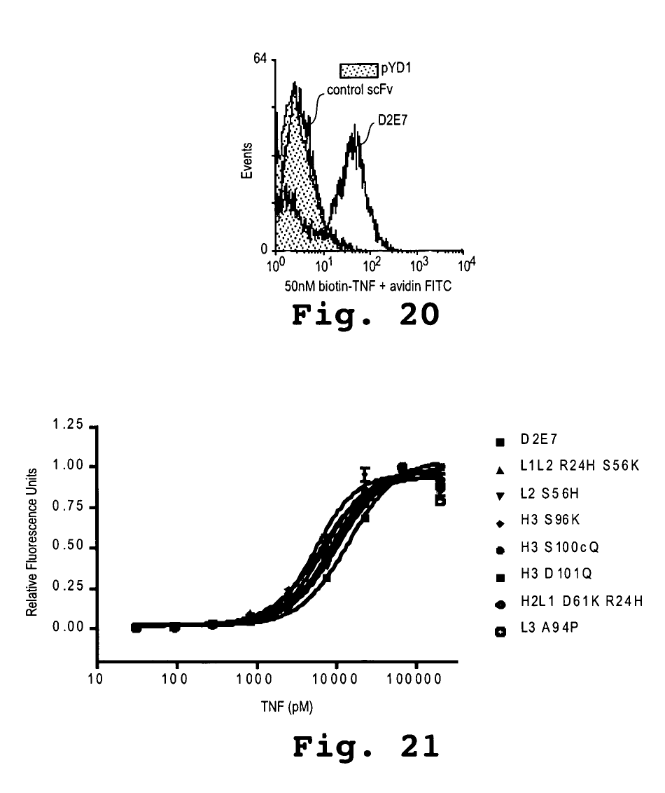

An aliquot of yeast cells (8×10⁵ cells in 40 μl) from the culture medium was centrifuged for 5 minutes at 2300 rpm. The supernatant was aspirated and the cell pellet was washed with 200 μl of ice cold PBS/BSA buffer (PBS/BSA 0.5% w/v). The cells were re-pelleted and supernatant removed before re-suspending in 100 μl of buffer containing the biotinylated TNFα (200 nM). The cells were left to bind the TNF-α at 20° C. for 45 minutes after which they were washed twice with PBS/BSA buffer before the addition and incubation with streptavidin-FITC (2 mg/L) for 30 minutes on ice. Another round of washing in buffer was performed before final re-suspension volume of 400 μl in PBS/BSA. The cells were then analysed on FACSscan (Becton Dickinson) using CellQuest software as per manufacturers directions. The FACS plot (FIG. 20) illustrates D2E7 scFv binding of biotinylated TNF-α and streptavidin FITC (the “green” line) producing a peak signal response a magnitude higher compared to signal from the empty vector pYD1 with biotinylated TNFα and streptavidin FITC (dark shaded area).

### EXAMPLE 5

### High throughput Library Screening for Antibody Affinity

A. Magnetic Sorting of TNF Binding (EC₅₀ FIG. 8)

FIG. 8 depicts a generalized scheme for enriching the TNF-α specific high affinity binding clones from the heterogeneous yeast scFv (LTM or WTM) library. After induction in Galactose media, the yeast cell library (10⁷) is resuspended in PBS/BSA buffer (total volume of 500 μl). Biotinylated TNF-α is added for a final concentration 50 nM and then incubated at 25° C. for 2-3 hours shaking. Yeast cells were pelleted and washed 3 times (500 μl) in. Afterwards, the yeast cells were resuspended in 300 μl ice cold PBS/BSA buffer of buffer with 1×10⁸ streptavidin coated magnetic beads (manufacturer) was added. The bead cell mixture was incubated on ice for 2 minutes with gentle mixing by inversion to form a binding complex consisting of yeast high-affinity scFv expressing cells, biotinylated TNF-α, and streptavidin coated magnetic beads. The tubes containing bound complexes were then applied to the magnetic column holder for 2 minutes. The supernatant was removed by aspiration, the column removed from the magnet holder, 300 μl ice cold PBS/BSA was added to resuspend bound complexes and column was placed back on the magnetic holder. The bound complexes were washed again in order to remove scFv clones of low-affinity and other non-specifically bound cells.

The tube was then removed from the magnetic holder whereupon 1 ml of Glucose select media was added and the recovered yeast cells to be incubated for 4 hours at 30° C. The magnet holder was re-applied to the culture tube to remove any remaining magnetic beads. The yeast culture was then grown in Glucose select media at 30° C. for 48 hours before scFv induction in Galactose select media. In the second selection round, TNF-α concentration was lowered from 50 nM to 0.5 nM. TNF-α binding, complex formation, yeast cell enrichment and re-growth were performed as described above. For the third selection round, the TNF-α concentration was further lowered to 0.1 nM.

TNF-α EC50 binding, or “fitness” from each round of enrichment was evaluated by FACS (Example 3 protocol). FIG. 9 illustrates that the initially transformed V_{H} LTM CDR3 yeast library with no prior selection (closed circles), the overall fitness in terms of percent binders (y-axis), clones expressing functional anti-TNF-α scFvs and their affinity, as measured by the TNF-α EC50 (x-axis) was inferior compared to the D2E7 wildtype. However, after just one round of selection (10 nM), the “fitness” curve (light triangles) improved in percent binders and the EC50 for TNF-α binding was in the same nM range as the D2E7 wild type. After the second selection round (0.1 nM), the enriched population (dark triangles) exhibited an overall “fitness” that nearly approached that of the D2E7 wild type (solid squares). The recovered yeast cells from the second round enrichment were then plated onto solid media in order to isolate single clones for individual binding analysis and sequence determination.

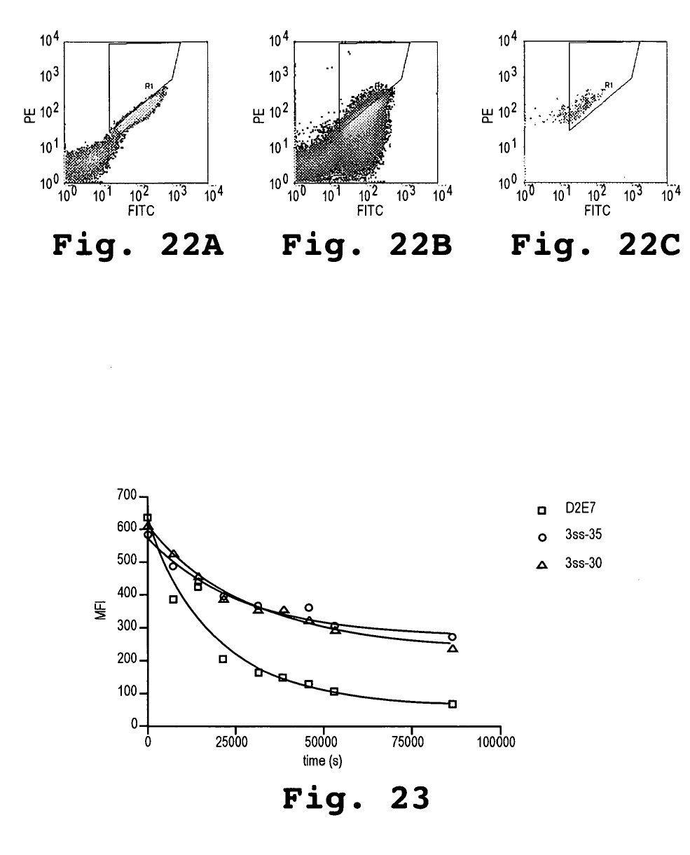

B. FACS Sorting of TNF scFv Library (FIGS. 11 and 22)

In an alternative methodology, the LTM yeast cell libraries were also enriched for high affinity anti-TNF-α scFv clones by FACS. Library construction, transformation, liquid media propagation and induction were carried out as above for EC50 determination. After scFv induction, the cells were incubated with biotinylated TNF-α at saturating concentrations (400 nM) for 3 hours at 25 C under shaking. After washing the cells, a 40 hour cold chase using unlabelled TNF-α (1 uM) at 25° C. was performed. The cells were then washed twice with PBS/BSA buffer, labeled with Streptavidin PE (2 mg/ml) anti-HIS-FITC (25 nM) for 30 minutes on ice, washed and re-suspended as described in Example 3. The D2E7 wild type was initially FACS analyzed to provide a reference signal pattern for FACS sorting of the yeast LTM library (FIG. 21, left panel). From the D2E7FACS plot, a selection gate (the R1 trapezoid) was drawn to obtain only those clones that expressed the scFv fusion (as detected by anti-HIS-FITC) and concomitantly would display a higher binding affinity to TNF-α (a stronger PE signal). FIG. 21 (middle panel) demonstrates that approximately 5% of the total LTM library was screened and selected by the R1 gate. After collection of these high anti-TNF-α scFv clones, a post sort FACS analysis (FIG. 21 right panel) was performed to confirm that >80% of the pre-screen anti-TNF-α scFv clones were within the predetermined criteria. The post FACS scFv clones were then grown in Glucose media at 30 C for 48 hours and then plated on solid media to isolate individual clones. Clones were grown in liquid Glucose select, re-induced in Galatose select and were analyzed for their EC50 and/or k_{off} characteristics as above.

### EXAMPLE 6

### Characterization of High-Affinity Antibodies

FACS Measurement of TNF-α EC₅₀ Binding:

A pre-determined amount of yeast cells (8×10⁵ cells in 40 μl) D2E7 scFvs (wild type, LTM, WTM clones) were incubated with 1:4 serial dilutions of biotinylated TNF-α (200 nM, 50 nM, 12.5 nM, 3.1 nM, 0.78 nM, and 0.19 nM final concentrations in a total volume of 80 μl) and incubated at 20° C. for 45 minutes followed by 5-10 minutes on ice. The yeast cells were washed 3 times and resuspended in 5 ml of PBS/BSA buffer. Streptavidin-PE (2 mg/ml) and αHIS-FITC (25 nM) was added to label the cells during an 30 minute incubation on ice. The αHIS-FITC antibody allowed monitoring of yeast cell surface scFv expression. Another round of washing was performed before re-suspending in 400 μl of PBS/BSA buffer. The labeled cells were then analyzed on FACSscan using CellQuest software.

FIG. 21 exemplifies a subset of improved clones relative to D2E7 in having a lower EC₅₀ values (their TNF-α binding curves have shifted to the left with respect to the D2E7 wild type solid square). Their relative EC₅₀ compared to D2E7 and fold increase are listed Table 1. For example, the clone H3 S96Q exhibited a 2.5 fold improvement in TNF-α binding. Nomenclature identification of this clone H3 S96Q, indicates that it was from a V_{H} CDR-H3 glutamine LTM single library. From FIG. 10 A, there were three independent V_{H} CDR-H3 H3 S96Q clones identified from the above EC₅₀ screen. In an example of identifying a Double LTM mutant, LTM L2 R24H S56K (FIG. 20 and FIG. 10B) illustrate that enhanced TNF-α binding only occurred when there was a synergistic interaction between these two CDR-L1 R24H and CDR-L2 S56K substitutions.

TABLE 1

| | | L1L2 | | | H3 | | H2L1 | |
|---|---|---|---|---|---|---|---|---|
| | | R24H | L2 | H3 | S100c | H3 | D61K | L3 |
| | D2E7 | S56K | S56H | S96K | Q | D101Q | R24H | A94P |
| Relative | 1.0 | 0.47 | 0.72 | 0.40 | 0.48 | 0.71 | 0.61 | 0.57 |
| EC₅₀ | | | | | | | | |
| Fold | | 2.1 | 1.4 | 2.5 | 2.1 | 1.4 | 1.6 | 1.7 |
| better | | | | | | | | |
| than | | | | | | | | |
| D2E7 | | | | | | | | |

### EXAMPLE 7

### High throughput Library Screening for Enhanced K_{off}

A. Individual scFv Clones:

From the FACS sorter, the pre-sorted clones were then grown overnight in Glucose select media and then plated on solid media to isolate single colonies.

From a single colony liquid cultures of clones were grown in Glucose select media at 30° C. with shaking for 48 hours. The cells were then pelleted and resuspended in Galactose select media for OD time period. Because the FACS pre-sort enriches (by approximately 80%) but does not eliminate all undesirable clones, it is necessary to characterize the EC50 of the isolated clones to eliminate those that display binding values inferior to D2E7 (as detailed in the procedure of Example 3). Those isolates with comparable or superior EC₅₀ values were then selected for further analysis.

Pulse: Yeast cells (approximately 5×10⁶) after induction in Galactose select media, were pelleted and re-suspended in PBS/BSA buffer (1 ml). Biotinylated TNF-α (400 nM final concentration) was then added to the re-suspended cells and allowed to incubate or 2 hours at 25° C. on a nutator for continuous gentle mixing.

Chase: The biotinylated-TNF-□ and yeast cell mixture was washed and re-suspended in PBS/BSA buffer. Unlabelled TNFα was then added (to a final concentration of 1 μM) and yeast cell mixture was further incubated for 24 hours at 25° C. with sample aliquots being taken every two hours for the next 24 hours. The cell mixtures were washed and re-suspended in chilled PBS/BSA buffer and staining antibody α-SA PE (2 μg/ml) added. After incubation for 30 minutes on ice with periodic mixing, the cell mixture was then twice washed and analyzed by FACS as above.

From these K_{off} assays, FIG. 23 demonstrates the effect of two clones, 3ss-35 and 3ss-30 having a higher relative K_{off} compared to D2E7. In other words, when exchanging the bound biotinylated TNF-α for the unlabelled TNF-α during the 24 hour sampling period, 3ss-35 and 3ss-30 released the previously bound biotinylated TNF-α at a much slower rate (open circles and triangles respectively in FIG. 23). D2E7 wild type, (open squares FIG. 23) in contrast, exhibited a much sharper decrease in MFI over the first 8 hours. From the various single LTM libraries in the V_{H} and V_{L} CDRs,

FIG. 12A and 12B enumerate the results of these LTM k_{off} assays. For example, there were seven independent V_{H} CDR-H1 D31Q LTM single clones and eleven V_{H} CDR-H1 Y32S LTM clones indicating that these two respective substitutions have a profound impact on the k_{off} rate in the D2E7 scFv.

B. Beneficial Library (Mixed Mutation) Construction

FIGS. 13A and 13B lists all the beneficial D2E7 CDR mutations discovered thus far and is a aggregate of the sequence clones isolated from both the equilibrium (EC₅₀ FIGS. 10A and 10B) and kinetic assays (K_{off} FIGS. 12A, 12B). For example, FIG. 13B composite sequence lists H₁₆₄ S/Y/K₁₆₇ K₁₆₈L/K₁₆₉ as the CDR L1 beneficial mutations in which the H₁₆₄ mutation was primarily identified by equilibrium assays whereas the K₁₆₈K/L₁₆₉ mutations were mainly identified from K_{off} assays. From these composite CDR mutations, degenerate oligonucleotides were designed to incorporate all the beneficial mutations in each CDR.

The sequence of the 6 degenerate CDR beneficial mutation oligonucleotides are listed in SEQ ID NOS: 46-51. For example, the CDR L1 beneficial mutation oligonucleotide coded for H₁₆₄ A₁₆₅ S₁₆₆S/Y/K/Q₁₆₇ G/K₁₆₈L/K/I₁₆₉ R₁₇₀ N₁₇₁ Y₁₇₂ L₁₇₃ A₁₇₄. Two separate libraries were constructed, one composed of H1, H2, and H3 beneficial mutations (a triple V_{H} CDRlibrary) and the other library composed of the triple L1, L2, and L3 beneficial mutations (triple V_{L} CDR library). The incorporation of multiple degenerate CDRs into one was detailed above in Example 2 (FIGS. 16A-16D and 17A-17D). Briefly, for example, CDR H2 was first mutated by the mixed mutation oligonucleotides to create a “single” mixed mutation library. The CDR H2 mixed mutation library would then serve as templates to incorporate the degenerate CDR H1 mixed mutation oligonucleotides to create a “double” CDR H1 H2 mixed mutation library. The CDR H1 H2 mixed mutation library in turn, serves as the template for the CDR H3 mixed mutation oligonucleotides to create the “triple” CDR H1 H2 H3 mixed mutation library. The triple CDR library light chain variants were created in an analogous manner. Each triple CDR V_{H} and V_{L} library had a diversity of approximately a million variants. Resulting variants from these triple libraries were however, selected only be k_{off} assays.

C. Beneficial Library (Mixed Mutation) Clones

FIGS. 24A and 24B identify mixed mutation clones, showing 63 unique sequences for scFv anti-TNF-α clones recovered from the mixed mutation WTM libraries screened by K_{off} assays. Overall, the K_{off} clones recovered had incorporated substitutions in all six CDRs and varying degrees of mixed mutation introduction within each CDR. For example, the triple V_{L} library clone LB-E2 exhibited a high relative (5.3×) K_{off} incorporated beneficial mixed mutation combinations of H₁₆₄ R₁₆₇, R₁₆₈and L₁₆₉ within CDR L1, S₁₉₃, F₁₉₄, L₁₉₅, Q₁₉₆ in CDR L2 and beneficial mutation combination of D₂₀₇ and P₂₀₉ in CDR L3. V_{H} triple library clones also demonstrated multiple mixed mutation beneficial combinations V_{H} CDRs. For example, the clone HB-B1, there was mixed mutation combination preference of Q₃₁Y₃₂ in CDR H1 in conjunction with Q₁₀₃ Q₁₀₉ S₁₁₂ in CDR H3.

### EXAMPLE 8

### BiaCore Analysis of High-Affinity Clones

pBAD Fab Construction

The scFv genes for D2E7 and those clones identified from the above K_{off} screens characterized as affinity-enhanced, were excised from pYD1 and sub-cloned into pBAD E. coli expression vector (Invitrogen pBAD expression system).

A. E. coli pBAD expression for production of soluble antibodies

Competent E. coli host cells were prepared as per manufacturer's instructions (Invitrogen pBAD expression system). Briefly, 40 μl LMG 194 competent cells and 0.5 μl pBAD scFv construct (approximately 1 μg DNA) was incubated together on ice for 15 minutes after which, a one minute 42° C. heat shock was applied. The cells were then allowed to recover for 10 minutes at 37° C. in SOC media before plating onto LB-Amp plates and 37° C. growth overnight. Single colonies were picked the next day for small scale liquid cultures to initially determine optimal L-arabinose induction concentrations for scFv production. Replicates of each clone after reaching an OD₆₀₀=0.5 were test induced with serial (1:10) titrations of L-arabinose (0.2% to 0.00002% final concentration) after overnight growth at room temperature. Test cultures (1 ml) were collected, pelleted and100 μl 1× BBS buffer (10 mM, 160 mM NaCl, 200 mM Boric acid, pH=8.0) added to resuspend the cells before the addition of 50 μl of Lysozyme solution for 1 hour (37° C.). Cell supernatants from the lysozyme digestions were collected after centrifugation, and MgSO₄ was added to final concentration 40 mM. This solution was applied to PBS pre-equilibrated Ni-NTA columns. His-tagged bound scFv samples were twice washed with PBS buffer upon which elution was accomplished with the addition of 250 mM Imidazole. Soluble scFvs expression was then examined by SDS-PAGE.

Purification of scFv from Large Scale E. coli Culture:

After determination of optimal growth conditions, large scale (volume) whole E. coli cell culture pellets were collected by centrifugation after overnight growth at 25° C. The pellets were then re-suspended in PBS buffer (0.1% tween) and subjected to 5 rounds of repeated sonication (Virtis Ultrasonic cell Disrupter) to lyse the bacterial cell membrane and release the cytoplasmic contents. The suspension was first clarified by high speed centrifugation to collect the supernatant for further processing. This supernatant was applied to PBS pre-equilibrated Ni-NTA columns. His-tagged bound scFv samples were twice washed with PBS buffer upon which elution was accomplished with the addition of 250 mM Imidazole. The pH of the supernatant was then adjusted to 5.5 with 6M HCl and before loading onto a SP Sepharose HP cation exchange column (Pharmacia). The scFv was eluted a salt (NaCl) gradient and fraction concentrations containing the scFv were determined by optical density at 280 nm and verified by PAGE. Fractions containing scFvs were then pooled and dialyzed with PBS.

Biacore Binding Analysis:

The TNF-α binding affinities (KD=k_{d}/kₐ=k_{off}/kₒₙ) of the scFv fragments were calculated from the resultant association (kₐ=kₒₙ) and dissociation (k_{d}=k_{off}) rate constants as measured using a BIAcore-2000 surface plasmon resonance system (BIAcore, Inc). To avoid valency problems due to the trimeric state of TNFα, the ligand was immobilized on the BIAcore chip sensor surface in effect, allows monitoring of the monomeric scFv binding from the flowed solution. BIAcore biosensor chip were activated for covalent coupling of TNF-α using N-ehtyl-N′-(3-dimethylaminopropyl)-carbo-diimide hydrochloride (EDC) and N-hydrosuccinimide (NHS) according to manufacturer's instructions. A solution of ethanolamine was injected as a blocking agent.

For the flow analysis, anti-TNF-α scFv were diluted into 20 mM Hepes buffered Saline pH 7.0 and diluted to approximately 50 nM. Aliquots of anti-TNF-α scFvs were injected at a flow rate of 2 ul/minute. For kinetic measurements, scFvs were injected at a flow rate of 10 ul/min. Dissociation was observed in running buffer without dissociating agents. The kinetic parameters of the binding reactions were determined using BIAevaluation 2.1 software.

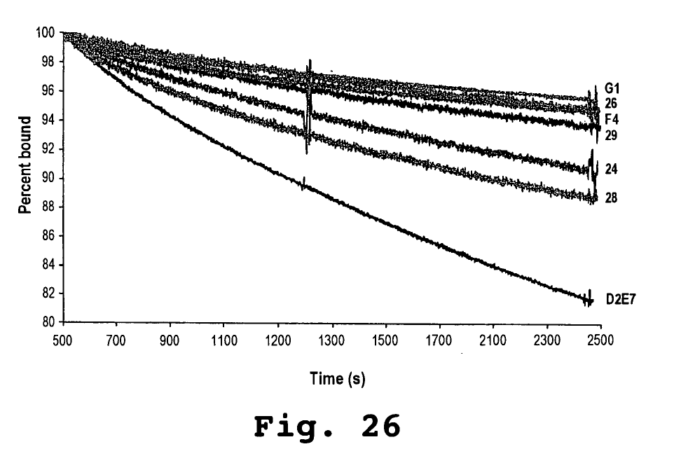

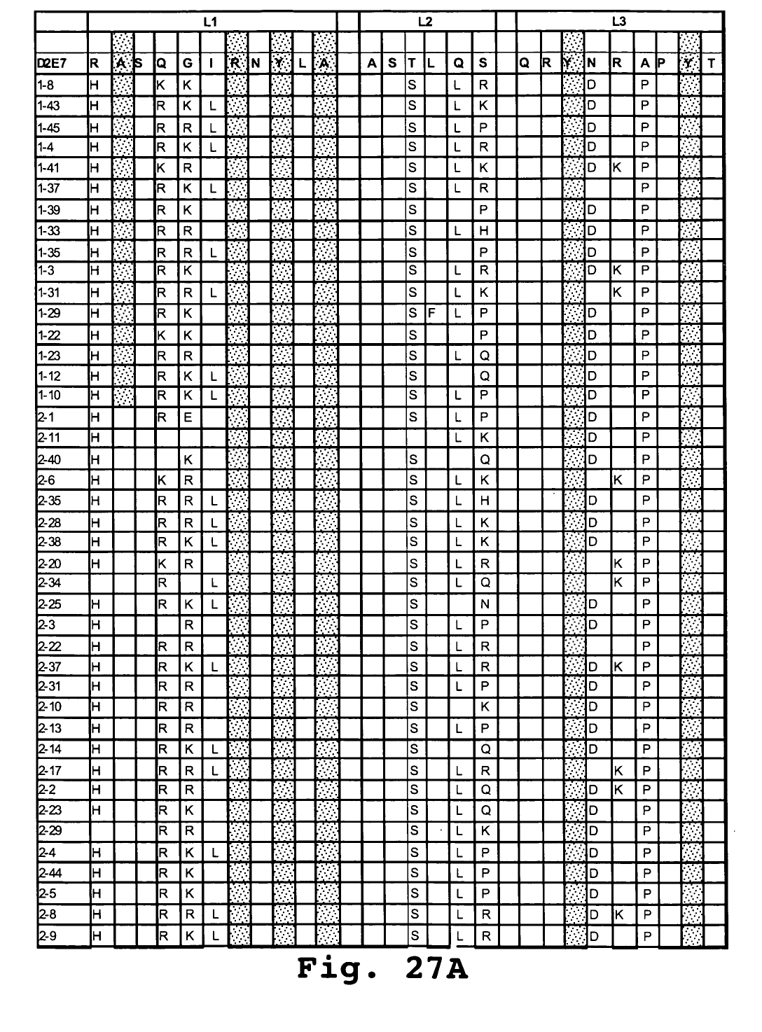

FIG. 25 displays BIAcore scFv results from the reference D2E7 anti-TNF-α and six affinity enhanced K_{off} clones. It is evident from these plots that D2E7, in comparison with all six clones, displays a noticeably sharper decaying slope indicative of a faster K_{off}. In comparison of kₒₙ values, most of the clones were relatively comparable to D2E7 although one, Fab 26-1, demonstrated a 1.6× slower binding rate. When these dissociation profiles were normalized and over-layed together (FIG. 26), it is clear that D2E7 dissociates from the immobilized bound TNF-α at a faster rate. For example, nearing the end of the monitored interval at 2500 seconds, only 80% of D2E7 was bound whereas all six clones still displayed greater than 90% binding. In fact, the best clone G1 was exhibited 96% binging. Compared to D2E7 wild-type, this clone G1 was exhibited more than 8 fold binding affinity (KD 247 pM vs. 30 μM respectively).

TABLE 2
The table below provides the rate constants determined for
each anti-TNFα scFv interacting with the TNFα surface.
The affinity (K_{D}) is reported in units of pM.

| scFv | kₐ | error | k_{d} | error | K_{D} (pM) |
|---|---|---|---|---|---|
| D2E7 | 4.66E+5 | 2E+3 | 1.15E−4 | 2.E−7 | 247 |
| 29 | 2.80E+5 | 1E+3 | 3.00E−5 | 9E−8 | 107 |
| 24 | 5.30E+5 | 4E+3 | 5.00E−5 | 3E−7 | 94 |
| 26 | 3.34E+5 | 1E+3 | 2.70E−5 | 9E−8 | 81 |
| 28 | 4.30E+5 | 2E+3 | 5.20E−5 | 4E−7 | 121 |
| F4 | 5.80E+5 | 7E+3 | 2.20E−5 | 2E−7 | 38 |
| G1 | 4.90E+5 | 5E+3 | 1.49E−5 | 3E−7 | 30 |

Overall, as shown in Table 2, the association rate constants, kₐ, for all examined clones varied by 2.1 fold (2.8×10⁵ to 5.8×10⁵), whereas the dissociation rate, k_{d} improved by 7.7 fold (1.15×10⁻⁴ to 1.49×10⁻⁵). Thus, the enhanced affinity shown by these anti-TNF-α clones is contributed mainly by their improved dissociation rate (k_{d)} kinetics. ehtyl-N′9

### In vitro Functional Properties of High-Affinity Clones in Neutralizing the Cytotoxic Effects of TNF-α in Actinomycin Treated L929 Cells

The biological activity of the affinity enhanced CBM clones was measured using a TNF-α induced L929 cell cytotoxicity assay. Murine L929 cells after brief co-treatment with Actinomysin D are susceptible to TNF-α mediated cytotoxicity. If however, the soluble TNF-α is co-incubated with anti-TNF-α antibodies, the antibody bound cytokine unable to bind the TNF receptor and the cytotoxicity is neutralized. For a given concentration of anti-TNF-α antibody, the degree of cytotoxicity protection afforded by the anti-TNF-α antibody is therefore dependent upon its binding affinity for TNF-α. To determine the IC₅₀, various TNF-α and antibody concentrations were co-incubated for 24 hours after which, a calorimetric metabolic dye was added to determine the extent of cell death and antibody mediated protection by measuring the resultant optical density generated by the substrate conversion in living cells.

Cell Culture:

L929 cells were propagated in the following growth medium: Minimal Essential Medium (Eagles), supplemented with 2 mM L-glutamine, and Earle's BSS adjusted to contain 1.5 g/L sodium bicarbonate, 0.1 mM non-essential amino acids, and 1.0 mM sodium pyruvate, 10% FBS, 50 μg/mL gentamycin and cultivated in incubators at 37° C. in an atmosphere of 5% CO₂. Before attaining confluence, L929 cell populations were sub-cultured at a ratio of 1:4 three times a week to maintain cells in the logarithmic phase of growth.

Neutralization Assay:

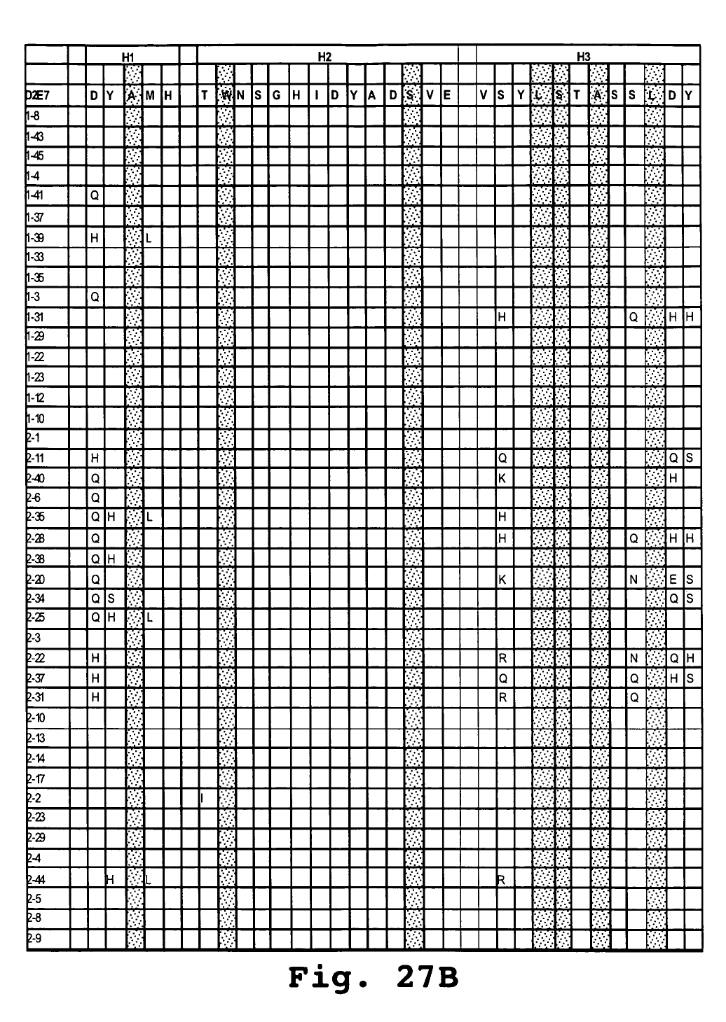

The neutralization assay that was performed was a modification of a procedure developed by Doring et al, (Molecular Immunology, 31:1059-1067 (1994)). In brief, L929 cells were plated 35,000 cells per well in a 96-well micro titer plate for overnight growth. The next day, the following six antibody drugs were serially diluted so that the final concentrations in the well would be as follows: Positive control Humira (IgG1) and D2E7 (scFv): 8100 pM, 2700 pM, 900 pM, 300 pM, 100 pM, 33.3 pM, 11.1 pM, 3.7 pM, 1.23 pM, 0.411 pM; CBM affinity enhanced clone Al (in scFv format): 1620 pM, 540 pM, 180 pM, 60 pM, 20 pM, 6.67 pM, 2.22 pM, 0.741 pM, 0.247 pM, 0.082 pM; CBM affinity enhanced clones 2-44-2, 1-3-3, 2-6-1 (all in scFv format): 810 pM, 270 pM, 90 pM, 30 pM, 10 pM, 3.33 pM, 1.11 pM, 0.370 pM, 0.123 pM, 0.0411 pM. The A1 sequence has the D2E7 mutations CDRH1:D31Q, CDRH3:S99P, and CDRL1:G28E. The 2-44-2, 1-3-3 and 2-6-1 antibodies have the mutations shown in FIG. 27B for 2-44, 1-3, and 2-6, respectively.

Given the higher affinity of the anti-TNF-α antibodies, CBM clones were started with dilutions tenfold lower, since preliminary experiments showed that if the CBM clone concentrations were of similar concentrations with the positive control Humira and D2E7, adding TNF-α at the IC₅₀ value would not induce cytotoxicity. The diluent used for the antibody serial dilutions was the above MEM growth media. For the neutralization assay in the replicate wells of the above antibody control and clone dilutions, TNF-α was then added to yield two different final concentrations (175 pg/mL and 350 pg/mL). Therefore, one set of the antibody dilutions (e.g. 810 to 0.0411 pM) was incubated at a final TNF-α concentration of 175 pg/mL while another antibody dilution (e.g. 810 to 0.0411 pM) was incubated at 350 pg/mL TNF-α. To allow complex formation, these TNF-α and antibody co-incubations were performed at room temperature for 30 minutes prior to their addition to the cell culture plates.

As a negative binding control, an aliquot from each of the six test antibodies was boiled for 10 minutes, placed on ice for a few minutes then centrifuged (13,000 g) at 4° C. for 5 minutes to remove any precipitated material. One dilution concentration of the boiled, denatured antibodies was then co-incubated with TNF-α (175 pg/mL and 350 pg/mL) for 30 minutes at room temperature.

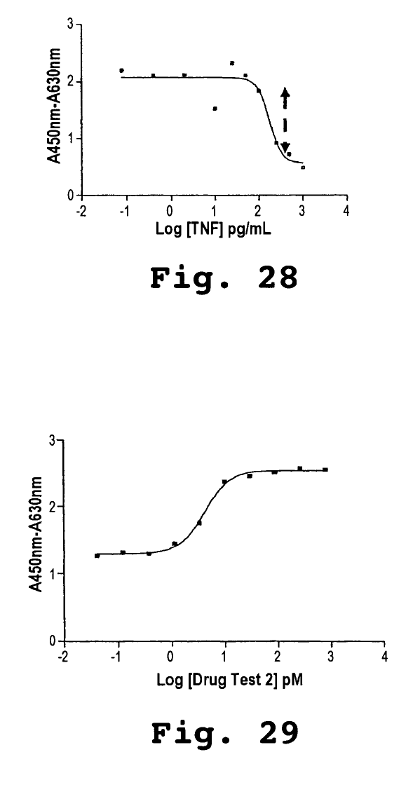

Prior to co-incubation of TNF-α and one of the test antibodies, the overnight media was aspirated from the L929 cell cultures and replaced with media containing 10% heat-inactivated serum and 1 μg/mL Actinomycin D. Exposure to Actinomycin D was no longer than 5-15 minutes prior to the addition of the TNF-α and antibody co-incubations. On the day that the neutralization experiments were run, a control TNF-α dose response curve was performed on a separate plate of L929 cells to ensure that the drug experiments are within the IC50 of cytotoxicity. The following TNF-α concentrations were used for the dose response curve: 0.08 pg/mL, 0.4 pg/mL, 2 pg/mL, 10 pg/mL, 25 pg/mL, 50 pg/mL, 100 pg/mL, 250 pg/mL, 500 pg/mL, and 1000 pg/mL. The TNF-α and antibody treated L929 cells were subsequently incubated for 20-24 hours at 37° C. The following day, a 1/10 volume ratio of WST-1 cell proliferation reagent was added to each well and the cells were allowed another 4 hours of incubation at 37° C. The introduced WST-1 reagent is taken in by the cell whereupon its' metabolized product causes an increase in OD 450 nm absorbance. Following WST-1 incubation, the culture plate was removed and placed upon a microplate reader where the absorbance at OD 450 nm was read and with a reference of 630 nm on a Wallac Victor2 plate reader. From the resulting plots, the IC₅₀s were then determined by using Prism version 3.02 software. From the TNF control dose response experiments, it can be seen that greater levels of cytotoxicity through increasing TNF concentration exposure will result in decreased OD 450 nm readings (FIG. 28).

Determination of the IC₅₀ of TNF-α Treated L929 Cells

Table 3 and the associated FIG. 28 plot is an example of the OD 450 nm readings obtained in determining the IC₅₀ of TNF-α treated L929 cells. A standard curve window of TNF concentrations (indicated by double headed arrow in FIG. 28) for the neutralization assay was determined through a series of repeated IC₅₀ experiments. It was ascertained that the anti-TNF-α antibody co-incubations would therefore be conducted in two final TNF-α concentrations of 175 pg/mL and 350 pg/mL. Protection from cytotoxicity by anti-TNF-α antibody mediated TNF-α neutralizations would then be most effectively reflected between the upper and lower ranges of the 175 to 350 pg/mL window.

TABLE 3
Raw 450 nm-A630 nm absorbance data: TNF-α curve.

| [TNF- | | | | | | | | Log |
|---|---|---|---|---|---|---|---|---|
| α] | Well | Well | Well | Well | Aver- | | | [TNF-α] |
| pg/mL | 1 | 2 | 3 | 4 | age | SD | % CV | pg/ml |
| 0 | 2.409 | 2.422 | 2.378 | 2.415 | 2.406 | 0.019 | 0.80 | NA |
| 0.08 | 2.402 | 2.018 | 2.257 | 2.111 | 2.197 | 0.168 | 7.66 | −1.10 |
| 0.4 | 2.330 | 1.973 | 2.263 | 1.891 | 2.114 | 0.215 | 10.17 | −0.40 |
| 2 | 2.197 | 2.140 | 2.161 | 1.990 | 2.122 | 0.091 | 4.31 | 0.30 |
| 10 | 1.749 | 1.071 | 2.088 | 1.222 | 1.533 | 0.471 | 30.75 | 1.00 |
| 25 | 1.722 | 1.767 | 1.807 | 1.680 | 1.744 | 0.055 | 3.15 | 1.40 |
| 50 | 1.913 | 1.470 | 1.715 | 1.241 | 1.585 | 0.292 | 18.43 | 1.70 |
| 100 | 1.666 | 1.037 | 1.403 | 1.419 | 1.381 | 0.259 | 18.78 | 2.00 |
| 250 | 1.196 | 0.804 | 0.894 | 0.817 | 0.928 | 0.183 | 19.73 | 2.40 |
| 500 | 0.923 | 0.605 | 0.686 | 0.678 | 0.723 | 0.138 | 19.10 | 2.70 |
| 1000 | 0.601 | 0.427 | 0.491 | 0.449 | 0.492 | 0.077 | 15.73 | 3.00 |
| Neg | 2.506 | 2.515 | 2.519 | 2.446 | 2.496 | 0.034 | 1.37 | NA |
| control | | | | | | | | |

Neutralization of the Cytotoxic Effect of TNF-α on L929 Cells

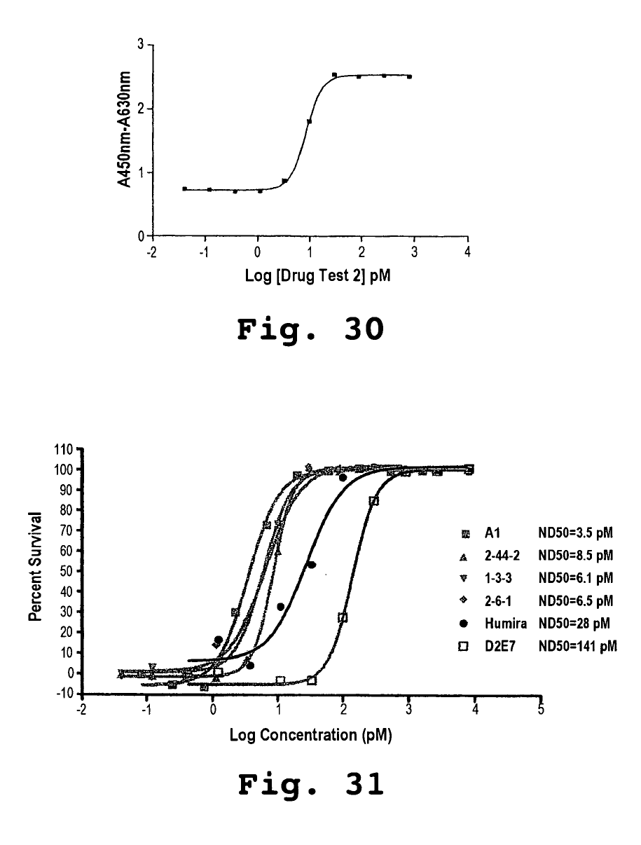

Comparative neutralization experiments with four of the CBM affinity enhanced anti-TNF-α clones and the positive control anti-TNF-α Humira (IgG1) and D2E7 (scFv) were performed on the same day to eliminate the typical day to day variability. The TNF-α neutralization results for CBM clone 2-44-2, and representative of the other CBM experimental clones, are shown in Tables 4 and 5 and associated graphical plots FIGS. 29 and 30 for TNF-α concentrations of 175 pg/mL and 350 pg/mL respectively. The results also indicate that pre-boiling the anti-TNF-α CBM clone prior to TNF-α co-incubation effectively abolishes the neutralization effect by the antibody. The OD 450 nm readings show that the boiled antibody and TNF-α co-incubations, the L929 cells were unable to metabolize the WST-1 substrate.

For CBM clone 2-44-2 (labeled as test drug 2 in the FIGS. 29 and 30), the IC50 neutralization was 4.21 pM and 8.54 pM for the 175 pg/mL and the 350 pg/mL TNF-α concentrations respectively. The mean of the neutralization response for both TNF-α concentrations was therefore 6.38 pM.

TABLE 4
Raw 450 nm-A630 nm absorbance data:
dose response 175 pg/mL TNF-α

| [Test | | | | | | | | Log |
|---|---|---|---|---|---|---|---|---|
| 2] | Well | Well | Well | Well | Aver- | | | [Test |
| pM | 1 | 2 | 3 | 4 | age | SD | % CV | 2] pM |
| 0 | 0.925 | 0.793 | 0.670 | 0.626 | 0.754 | 0.135 | 17.85 | NA |
| 0.0411 | 1.407 | 1.225 | 1.299 | 1.132 | 1.266 | 0.116 | 9.19 | −1.39 |
| 0.123 | 1.399 | 1.256 | 1.300 | 1.309 | 1.316 | 0.060 | 4.56 | −0.91 |
| 0.3703 | 1.743 | 1.140 | 1.107 | 1.193 | 1.296 | 0.300 | 23.18 | −0.43 |
| 1.11 | 1.505 | 1.255 | 1.492 | 1.531 | 1.446 | 0.128 | 8.85 | 0.05 |
| 3.33 | 1.774 | 1.543 | 1.970 | 1.721 | 1.752 | 0.176 | 10.03 | 0.52 |
| 10 | 2.355 | 2.367 | 2.368 | 2.382 | 2.368 | 0.011 | 0.47 | 1.00 |
| 30 | 2.452 | 2.471 | 2.461 | 2.452 | 2.459 | 0.009 | 0.37 | 1.48 |
| 90 | 2.525 | 2.524 | 2.492 | 2.504 | 2.511 | 0.016 | 0.64 | 1.95 |
| 270 | 2.591 | 2.566 | 2.555 | 2.579 | 2.573 | 0.016 | 0.61 | 2.43 |
| 810 | 2.561 | 2.549 | 2.538 | 2.561 | 2.552 | 0.011 | 0.43 | 2.91 |

| | Well 1 | Well 2 | Average |
|---|---|---|---|
| Boiled Drug | 0.663 | 0.638 | 0.650 |

TABLE 5
Raw 450 nm-A630 nm absorbance data: TNF-α.

| | | | | | | | | Log |
|---|---|---|---|---|---|---|---|---|
| [Test | Well | Well | Well | Well | Aver- | | | [Test |
| 2]pM | 1 | 2 | 3 | 4 | age | SD | % CV | 2] pM |
| 0 | 0.457 | 0.454 | 0.444 | 0.432 | 0.447 | 0.011 | 2.55 | NA |
| 0.0411 | 0.753 | 0.754 | 0.726 | 0.747 | 0.745 | 0.013 | 1.76 | −1.39 |
| 0.123 | 0.805 | 0.719 | 0.726 | 0.682 | 0.733 | 0.052 | 7.08 | −0.91 |
| 0.3703 | 0.739 | 0.726 | 0.688 | 0.670 | 0.706 | 0.032 | 4.57 | −0.43 |
| 1.11 | 0.714 | 0.720 | 0.702 | 0.728 | 0.716 | 0.011 | 1.56 | 0.05 |
| 3.33 | 0.900 | 0.925 | 0.906 | 0.780 | 0.878 | 0.066 | 7.52 | 0.52 |
| 10 | 1.876 | 1.910 | 1.734 | 1.742 | 1.815 | 0.091 | 4.99 | 1.00 |
| 30 | 2.569 | 2.563 | 2.556 | 2.494 | 2.545 | 0.035 | 1.36 | 1.48 |
| 90 | 2.539 | 2.537 | 2.514 | 2.480 | 2.517 | 0.028 | 1.09 | 1.95 |
| 270 | 2.511 | 2.527 | 2.619 | 2.450 | 2.527 | 0.070 | 2.76 | 2.43 |
| 810 | 2.531 | 2.524 | 2.558 | 2.461 | 2.518 | 0.041 | 1.62 | 2.91 |

Overall, the average IC50 for the TNF-α dose response curve was 248 pg/mL, well within the parameters of the values chosen by Bioren for the assay (175 pg/mL and 350 pg/mL). From their respective TNF-α neutralization assays, the average IC50 of affinity enhanced anti-TNF-α CBM clones (A1, 2-44-2, 1-3, 2-6-1) was determined to be approximately 5.11 pM (FIG. 31). These show that the anti-TNF-α CBM clones and are 4.5 fold and 20 fold higher than anti-TNF-α positive controls Humira and D2E7 respectively in protecting L929 cells from of TNF-α induced cytotoxicity (Table 6).

TABLE 6
Comparative IC50 summary table of neutralizing
anti-TNF-α antibodies

| | Average IC50 |
|---|---|
| Drug | (pM) |
| A1 | 3.28 |
| 2-44-2 | 6.38 |
| 1-3-3 | 5.25 |
| 2-6-1 | 5.53 |
| Humira | 23 |
| D2E7 | 105 |
| TNF-α dose | 248 pg/mL |
| response | |
| curve | |

Although the invention has been described with reference to particular embodiments and examples, it will be appreciated that various modifications and other applications may be made without departing from the spirit of the invention. For example, the selection of representative amino acids employed in LTM and WTM may be modified in a variety of ways that preserve the representation of basic physiocochemical properties of the 20 basic amino acids. Similarly, different antibody formats, and different reference sequences may be used. Instead of starting with all “human-derived” CDRs, for example, one or more of HV or HL chain CDRs could be based on mouse CDR sequence for the corresponding mouse anti-anti-TNF-α antibody sequence. Such a construction would be expected to provide additional structure-activity relationship information on the affect of amino acid sequence and binding activity.

Sequence Listing

SEQ ID NO: 1: (the amino acid sequence for D2E7 scFv antibody):

MEVQLVESGG GLVQPGRSLR LSCAASGFTF DDYAMHWVRQ
APGKGLEWVS AITWNSGHID YADSVEGRFT ISRDNAKNSL
YLQMNSLRAE DTAVYYCAKV SYLSTASSLD YWGQGTLVTV
SSGGGGSGGG GSGGGGSDIQ MTQSPSSLSA SVGDRVTITC
RASQGIRNYL AWYQQKPGKA PKLLIYAAST LQSGVPSRFS
GSGSGTDFTL TISSLQPEDVA TYYCQRYNRA PYTFGQGTKV
EIKAAAHHHH HHGEQKLISE EDL *

SEQ ID NO: 2: (the V_{L} amino acid sequence of D2E7 with all V_{L} CDR1-CDR3 mutations selected for enhanced affinity indicated as single or alternative-residue amino acids.)

DIQMTQSPSS LSASVGDRVT ITCX₁AS X₂X₃X₄R NYLAWYQQKP
GKAPKLLIYA X₅X₆X₇X₈X₉X₁₀GVPS RFSGSGSGTD FTLTISSLQP
EDVATYYCQ X₁₁ YX12X13X14X15X16X17FGQG TKVEIKAAAH
HHHHHGEQKL ISEEDL

X₁═R or H

X₂=L, Q, R, K or Y

X₃=G, E, R, S, Y or K

X₄═I, L or K

X₅=A or K

X₆=L, S or Y

X₇═S, A, K or T

X₈═F, P or L

X₉=Q, Y, K or L

X₁₀═S, Q, H, R, K, N or P

X₁₁═K or R

X₁₂═N or D

X₁₃═R, S, K, L, Q or D

X₁₄=A, P or K

X₁₅═P or Q

X₁₆═Y or Q

X₁₇=T or A

SEQ ID NO: 3: (the V_{L} CDR1 amino acid sequence with all mutations selected for enhanced affinity indicated as single or alternative-residue amino acids.)

X₁AS X₂X₃X₄RNYLA

X₁═R or H

X₂=L, Q, R, K or Y

X₃=G, E, R, S, Y or K

X₄═I, L or K

SEQ ID NO: 4: (the V_{L} CDR2 amino acid sequence with all mutations selected for enhanced affinity indicated as single or alternative-residue amino acids.)

X₁X₂X₃X₄X₅X₆

X₁=A or K

X₂=L, S or Y

X₃═S, A, K or T

X₄═F, P or L

X₅=Q, Y, K or L

X₆═S, Q, H, R, K, N or P

SEQ ID NO: 5: (the V_{L} CDR3 amino acid sequence with all mutations selected for enhanced affinity indicated as single or alternative-residue amino acids.)

QX₁YX₂X₃X₄X₅X₆X₇

X₁═K or R

X₂═N or D

X₃═R, S, K, L, Q or D

X₄=A, P or K

X₅═P or Q

X₆═Y or Q

X₇=T or A

SEQ ID NO: 6: (the amino acid sequence for D2E7 V_{H}).

EVQLVESGGG LVQPGRSLRL SCAASGFTFD DYAMHWVRQA
PGKGLEWVSA ITWNSGHIDY ADSVEGRFTI SRDNAKNSLY
LQMNSLRAE DTAVYYCAKV SYLSTASSLD YWGQGTLVTV S

SEQ ID NO: 7: (the V_{H} amino acid sequence of D2E7 with all V_{H} CDR1-CDR3 mutations selected for enhanced affinity indicated as single or alternative-residue amino acids.)

EVQLVESGGG LVQPGRSLRL SCAASGFTFD X₁ X₂A X₃HWVRQA
PGKGLEWVSA IX₄ X₅NSGHX₆ X₇Y X₈D X₉VEGRFTI
SRDNAKNSLY LQMNSLRAE DTAVYYCAK X₁₀ X₁₁X₁₂L X₁₃T
X₁₄X₁₅X₁₆X₁₇X₁₈

X₁₉WGQGTLVTV S

X₁=D, Q, Y or H

X₂═Y, S, or H

X₃=M or L

X₄=T, S, I, or A

X₅═W or Y

X₆═I, A, or H

X₇=D, K or S

X₈=A, S or K

X₉═S or P

X₁₀=A, K, S or V

X₁₁═S, K, Q, H, R, or T

X₁₂═Y, K, Q or H

X₁₃═S or P

X₁₄=A or S

X₁₅═S, D or P

X₁₆═S, Q or N

X₁₇=L or H

X₁₈=D, H, S or Q

X₁₉═Y, N, S, L, Q, or H

SEQ ID NO: 8: (the V_{H} CDR1 amino acid sequence with all mutations selected for enhanced affinity indicated as single or alternative-residue amino acids.)

X₁X₂AX₃H

X₁=D, Q, Y or H

X₂═Y, S, or H

X₃=M or L

SEQ ID NO: 9: (the V_{H} CDR2 amino acid sequence with all mutations selected for enhanced affinity indicated as single or alternative-residue amino acids.)

X₁X₂NSGHX₃X₄YX₅DX₆VE

X₁=T, S, I, or A

X₂═W or Y

X₃═I, A, or H

X₄=D, K or S

X₅=A, S or K

X₆═S or P

SEQ ID NO: 10: (the V_{H} CDR3 amino acid sequence with all mutations selected for enhanced affinity indicated as single or alternative-residue amino acids.)

X₁X₂X₃LX₄TX₅X₆X₇X₈X₉X₁₀

X₁=A, K, S or V

X₂═S, K, Q, H, R, or T

X₃═Y, K, Q or H

X₄═S or P

X₅=A or S

X₆═S, D or P

X₇═S, Q or N

X₈=L or H

X₉=D, H, S or Q

X₁₀═Y, N, S, L, Q, or H

SEQ ID NO: 11: the combinatorial coding sequences for the V_{L} CDR1

5′-CX₁T GCA TCT X₂X₃X₄ X₅X₆A X₇X₈A AGA AAT TAT
CTC GCA -3′

X₁=A or G

X₂=A, C, or T

X₃=A or G

X₄=G or T

X₅=A or G

X₆=A or G

X₇=A or C

X₈=A or T

SEQ ID NO: 12: the combinatorial coding sequences for the V_{L} CDR2

5′-GCC GCC TX₁T X₂CT TTX₃ CX₄A X₅X₆X₇-3′

X₁=A or C

X₂=A or T

X₃=A or T

X₄=A or T

X₅=A or C

X₆=A, C or G

X₇=T or G

SEQ ID NO: 13: the combinatorial coding sequences for the V_{L} CDR3

5′-CAA AGA TAC X₁AT AX₂A X₃CT CCA TAT ACA -3′

X₁=A or G

X₂=A or G

X₃=G or C

SEQ ID NO: 14: the combinatorial coding sequences for the V_{H} CDR1

5′- X₁A X₂ X₃ X₄T GCT X₅TG CAT-3′

X₁═C, G or T

X₂=G or T

X₃═C or T

X₄=A or C

X₅=A or C

SEQ ID NO: 15: the combinatorial coding sequences for the V_{H} CDR2

5′- ACA TAT AAT TCC GGT CAT ATT GAT TAC GCT GAC
TCT GTT GAG -3′

SEQ ID NO: 16: the combinatorial coding sequences for the V_{H} CDR3

5′- GTG X₁ X₂ X₃ TAC TTA TCA ACA GCT TCT X₄ X₅ X₆
CTA X₇A X₈ X₉ X₁₀ X₁₁-3′

X₁=A or C

X₂=A or G

X₃=G or T

X₄=A or C

X₅=A or G

X₆=G or T

X₇═C or G

X₈=G or T

X₉═C or T

X₁₀=A or C

X₁₁=G or T

SEQ ID NO: 17: the complete nucleotide sequence of D2E7 scFV antibody;

| | |
|---|---|
| 5′-ATG GAA GTT CAA TTG GTA GAA AGT GGT GGG | 30 |
| GGA TTA GTG CAA CCA GGT AGA TCT CTA AGG | 60 |
| CTT AGC TGT GCT GCA TCT GGG TTC ACC TTT | 90 |
| GAC GAT TAT GCT ATG CAT TGG GTC CGA CAA | 120 |
| GCG CCA GGA AAA GGT CTA GAG TGG GTT TCT | 150 |
| GCG ATA ACA TGG AAT TCC GGT CAT ATT GAT | 180 |
| TAC GCT GAC TCT GTT GAG GGT AGA TTT ACT | 210 |
| ATT TCC CGT GAT AAT GCT AAG AAC TCT TTG | 240 |
| TAC TTG CAG ATG AAT TCT TTA AGA GCA GAG | 270 |
| GAC ACC GCT GTA TAT TAC TGT GCA AAG GTG | 300 |
| TCT TAC TTA TCA ACA GCT TCT TCG CTA GAT | 330 |
| TAT TGG GGG CAA GGC ACT CTA GTC ACT GTT | 360 |
| AGT TCT GGT GGA GGC GGT TCT GGT GGA GGC | 390 |
| GGT TCG GGT GGC GGA GGT TCA GAT ATA CAA | 420 |
| ATG ACC CAA TCG CCT TCT AGC CTT TCT GCA | 450 |
| AGT GTT GGA GAC AGA GTA ACA ATA ACG TGT | 480 |
| CGT GCA TCT CAG GGT ATT AGA AAT TAT CTC | 510 |
| GCA TGG TAT CAA CAG AAG CCG GGT AAA GCA | 540 |
| CCT AAG CTG TTA ATT TAT GCC GCC TCA ACT | 570 |
| TTA CAA TCT GGT GTG CCT TCT AGG TTT AGT | 600 |
| GGT TCA GGT AGC GGT ACG GAT TTT ACT TTG | 630 |
| ACA ATT AGT TCA TTA CAG CCA GAA GAC GTT | 660 |
| GCA ACA TAT TAC TGT CAA AGA TAC AAT CGC | 690 |
| GCT CCA TAT ACA TTC GGT CAA GGT ACT AAA | 720 |
| GTC GAA ATC AAG GCG GCC GCT CAT CAC CAT | 750 |
| CAC CAT CAC GGA GAA CAA AAA TTG ATC TCA | 780 |
| GAG GAA GAT TTG TGA | 795 |

SEQ ID NO: 18: 5′ Bam HI Forward sense oligonucleotide for D2E7 scFv

5′-CGCGGATCCATGGAAGTTCAATTGGTAGAAAG-3′

SEQ ID NO: 19: 3′ Not I Reverse flanking oligonucleotide for D2E7 scFv

5′-ATGGTGGTGAGCGGCCGCCTTGATTTCGAC-3′

SEQ ID NO: 20: FR5 anti-sense oligonucleotide

5′- ATCGTCAAAGGTGAACCCAGATGCAGCACAGCTAAG-3′

SEQ ID NO 21: FR1 sense oligonucleotide

5′- ATGGAAGTTCAATTGGTAGAAAGTGGTGGGGGATTAGTG-3′

SEQ ID NO 22: FR2 anti-sense oligonucleotide

5′-ACCCAGGCTGTTCGCGGTCCTTTTCCAGATCTCACCCAA
AGACGCTAT-3′

SEQ ID NO 23: CDR H2 LTM oligonucleotides wildtype oligonucleotide

5′-gtagagtgggtttctgcgata-ACTTGGAATTCTGGTCATATTGATT
ATGCTGATTCTGTTGAA-ggtagatttactatttcccgt-3′

SEQ ID NO 24: CDR H2 LTM leucine oligonucleotides:

5′-gtagagtgggtttctgcgata-TTGTGGAATTCTGGTCATATTGATT
ATGCTGATTCTGTTGAA-ggtagatttactatttcccgt-3′

SEQ ID NO 25: CDR H2 LTM leucine oligonucleotides:

5′-gtagagtgggtttctgcgata-ACTTTGAATTCTGGTCATATTGATT
ATGCTGATTCTGTTGAA-ggtagatttactatttcccgt-3′

SEQ ID NO 26: CDR H2 LTM leucine oligonucleotides:

5′-gtagagtgggtttctgcgata-ACTTGGTTGTCTGGTCATATTGATT
ATGCTGATTCTGTTGAA-ggtagatttactatttcccgt-3′

SEQ ID NO 27: CDR H2 LTM leucine oligonucleotides:

5′-gtagagtgggtttctgcgata-ACTTGGAATTTGGGTCATATTGATT
ATGCTGATTCTGTTGAA-ggtagatttactatttcccgt-3′

SEQ ID NO 28: CDR H2 LTM leucine oligonucleotides:

5′-gtagagtgggtttctgcgata-ACTTGGAATTCTTTGCATATTGATT
ATGCTGATTCTGTTGAA-ggtagatttactatttcccgt-3′

SEQ ID NO 29: CDR H2 LTM leucine oligonucleotides:

5′-gtagagtgggtttctgcgata-ACTTGGAATTCTGGTTTGATTGATT
ATGCTGATTCTGTTGAA-ggtagatttactatttcccgt-3′

SEQ ID NO 30: CDR H2 LTM leucine oligonucleotides:

5′-gtagagtgggtttctgcgata-ACTTGGAATTCTGGTCATTTGGATT
ATGCTGATTCTGTTGAA-ggtagatttactatttcccgt-3′

SEQ ID NO 31: CDR H2 LTM leucine oligonucleotides:

5′-gtagagtgggtttctgcgata-ACTTGGAATTCTGGTCATATTTTGT
ATGCTGATTCTGTTGAA-ggtagatttactatttcccgt-3′

SEQ ID NO 32: CDR H2 LTM leucine oligonucleotides:

5′-gtagagtgggtttctgcgata-ACTTGGAATTCTGGTCATATTGAT
TTGGCTGATTCTGTTGAA-ggtagatttactatttcccgt-3′

SEQ ID NO 33: CDR H2 LTM leucine oligonucleotides:

5′-gtagagtgggtttctgcgata-ACTTGGAATTCTGGTCATATTGATT
ATGCTTTGTCTGTTGAA-ggtagatttactatttcccgt-3′

SEQ ID NO 34: CDR H2 LTM leucine oligonucleotides:

5′-gtagagtgggtttctgcgata-ACTTGGAATTCTGGTCATATTGATT
ATGCTGATTTGGTTGAA-ggtagatttactatttcccgt-3′

SEQ ID NO 35: CDR H2 LTM leucine oligonucleotides:

5′-gtagagtgggtttctgcgata-ACTTGGAATTCTGGTCATATTGATT
ATGCTGATTCTTTGGAA-ggtagatttactatttcccgt-3′

SEQ ID NO 36: CDR H2 LTM leucine oligonucleotides:

5′-gtagagtgggtttctgcgata-ACTTGGAATTCTGGTCATATTGATT
ATGCTGATTCTGTTTTG-ggtagatttactatttcccgt-3′

SEQ ID NO 37: FR4 anti-sense oligonucleotide

5′TCAGTTACTCACAAATCTTCCTCTGAGATCAATTTTTGTTCTCCGTGA
TGGTGATGGTGATGAGC-3′

SEQ ID NO 38: CDR H2 LTM aspartate oligonucleotide (Asp codon are bold)

5′-gtagagtgggtttctgcgata-GACTGGAATTCTGGTCATATTGATT
ATGCTGATTCTGTTGAA-ggtagatttactatttcccgt-3′

SEQ ID NO 39: CDR H2 WTM aspartate oligonucleotide:

5′-gtagagtgggtttctgcgata-RMT KRK RAT KMT GRT SAT
RWT GAT KAT GMT GAT KMT GWT GAW-ggtagatttactattt
cccgt-3′.

SEQ ID NO 41: CDR-H1 LTM leucine oligonucleotides:

5′-ctgggttcacctttgac-GCT TAT GCT ATG CAT -tgggtccg
acaagcgccag -3′

SEQ ID NO 42: CDR-H1 LTM leucine oligonucleotides:

5′-ctgggttcacctttgac-GAT GCT GCT ATG CAT -tgggtccg
acaagcgccag -3′

SEQ ID NO 43: CDR-H1 LTM leucine oligonucleotides:

5′-ctgggttcacctttgac-GAT TAT GCT ATG CAT -tgggtccg
acaagcgccag -3′

SEQ ID NO 44: CDR-H1 LTM leucine oligonucleotides:

5′-ctgggttcacctttgac-GAT TAT GCT GCT CAT -tgggtccg
acaagcgccag -3′

SEQ ID NO 45: CDR-H1 LTM leucine oligonucleotides:

5′-ctgggttcacctttgac-GAT TAT GCT ATG GCT -tgggtccg
acaagcgccag -3′

SEQ ID NO 46: CDR H1 beneficial mixed mutation oligonucleotide:

5′-ctgggttcacctttgac- BAK YMT GCT M TG CAT -tgggtc
cgacaagcgccag-3′

SEQ ID NO 47: CDR H2 beneficial mixed mutation oligonucleotide:

5′-ctagagtgggtttctgcgata- ACA TAT AAT TCC GGT CAT
ATT GAT TAG GCT GAG TCT GTT GAG -ggtagatttactattt
tcccgt-3′

SEQ ID NO 48: CDR H3 beneficial mixed mutation oligonucleotide:

5′-gtatattactgtgcaaag- GTG MRY TAC TTA TCA ACA GCT
TCT MRK CTA SAK YMK-tgggggcaaggcactctag-3′

SEQ ID NO 49: CDR L1 beneficial mixed mutation oligonucleotide:

5′-gacagagtaacaataacgtgt-CRT GCA TCT HRK RRA MWA
AGAAAT TAT CTC GCA -tggtatcaacagaagccg-3′

SEQ ID NO 50: CDR L2 beneficial mixed mutation oligonucleotide:

5′-cacctaagctgttaatttat-GCC GCC TMT WCT TTW CWA
MVK -ggtgtgccttctaggtttag-3′

SEQ ID NO 51: CDR L3 beneficial mixed mutation oligonucleotide:

5′-gacgttgcaacatattactgt-CAA AGA TAC RAT ARA SCT
CCA TAT ACA -ttcggtcaaggtactaaagtc-3′

SEQ ID NO: 52: Sense strand oligonucleotide S1

5′- ATG GAA GTT CAA TTG GTA GAA AGT GGT GGG GGA
TTA GTG -3′

SEQ ID NO: 53: Sense strand oligonucleotide S2

5′-CAA CCA GGT AGA TCT CTA AGG CTT AGC TGT GCT GCA
TCT G-3′

SEQ ID NO: 54: Sense strand oligonucleotide S3

5′-GG TTC ACC TTT GAC GAT TAT GCT ATG CAT TGG GTC
CGA CAA GCG CCA G -3′

SEQ ID NO: 55: Sense strand oligonucleotide S4

5′-GA AAA GGT CTA GAG TGG GTT TCT GCG ATA ACA TGG
AAT TCC GGT CAT ATT G-3′

SEQ ID NO: 56: Sense strand oligonucleotide S5

5′-AT TAC GCT GAC TCT GTT GAG GGT AGA TTT ACT ATT
TCC CGT GAT ATG-3′

SEQ ID NO: 57: Sense strand oligonucleotide S6

5′-CT AAG AAC TCT TTG TAC TTG CAG ATG AAT TCT TTA
AGA GCA GAG GAC ACC GCT G-3′

SEQ ID NO: 58: Sense strand oligonucleotide S7

5′- TA TAT TAG TGT GCA AAG GTG TCT TAC TTA TCA ACA
GCT TCT TCG CTA GAT TAT TGG GGG CAA GGC AC-3′

SEQ ID NO: 59: Sense strand oligonucleotide S8

5′-T CTA GTC ACT GTT AGT TCT GGT GGA GGC GGT TCT
GGT GGA GGC GGT TCG GGT GGC GGA GGT TC-3′

SEQ ID NO: 60: Sense strand oligonucleotide S9

5′-A GAT ATA CAA ATG ACC CAA TCG CCT TCT AGC
CTT TCT GCA AGT GTT GGA GAC AGA GTA ACA ATA
ACG TGT-3′

SEQ ID NO: 61: Sense strand oligonucleotide S10

5′- CGT GCA TCT GAG GGT ATT AGA AAT TAT CTC GCA
TGG TAT CAA GAG AAG CCG GGT AAA G-3′

SEQ ID NO: 62: Sense strand oligonucleotide S11

5′-CA CCT AAG CTG TTA ATT TAT GCC GCC TCA ACT TTA
CAA TCT GGT GTG CCT TCT AGG TTT AG-3′

SEQ ID NO: 63: Sense strand oligonucleotide S12

5′-T GGT TCA GGT AGC GGT ACG GAT TTT ACT TTG ACA
ATT AGT TCA TTA GAG CCA GAA G-3′

SEQ ID NO: 64: Sense strand oligonucleotide S13

5′- AC GTT GCA ACA TAT TAG TGT CAA AGA TAG AAT CGC
GCT CCA TAT ACA TTC GGT CAA GGT ACT AAA G-3′

SEQ ID NO: 65: Sense strand oligonucleotide S14

5′-TC GAA ATC AAG GCG GCC GCT CAT CAC CAT GAC CAT
CAC GGA GAA CAA AAA T3′

SEQ ID NO: 66: Sense strand oligonucleotide S15

5′-TG ATC TCA GAG GAA GAT TTG TGA GTA ACT GA-3′

SEQ ID NO: 67: Antisense strand oligonucleotide S1

AS1

5′- CCT TAG AGA TCT AGC TGG TTG CAC TAA TCC CCC
ACC ACT TTC TAC-3′

SEQ ID NO: 68: Antisense strand oligonucleotide S2

5′- GTC AAA GGT GAA CCC AGA TGC AGC ACA GCT
AAG- 3′

SEQ ID NO: 69: Antisense strand oligonucleotide S3

5′- AGA AAC CCA CTC TAG ACC TTT TCC TGG CGC TTG
TCG GAG CCA- 3′

SEQ ID NO: 70: Antisense strand oligonucleotide S4

5′-TAC CCT CAA CAG AGT CAG CGT AAT CAA TAT GAC CGG
AAT TCC ATG TTA TCG C-3′

SEQ ID NO: 71: Antisense strand oligonucleotide S5

5′-AAT TCA TCT GCA AGT ACA AAG AGT TCT TAG CAT TAT
CAC GGG AAA TAG TAA ATC3′

SEQ ID NO: 72: Antisense strand oligonucleotide S6

5′- CTT TGC ACA GTA ATA TAC AGC GGT GTC CTC TGC
TCT TAA AG- 3′

SEQ ID NO: 73: Antisense strand oligonucleotide S7

5′- AGA ACT AAC AGT GAC TAG AGT GCC TTG CCC
CCA- 3′

SEQ ID NO: 74: Antisense strand oligonucleotide S8

5′-ATT GGG TCA TTT GTA TAT CTG AAC CTC CGC CAC CCG
AAC CGC CTC CAC CAG AAC CGC CTC CAC C-3′

SEQ ID NO: 75: Antisense strand oligonucleotide S9

5′- GTC TCC AAC ACT TGC AGA AAG GCT AGA AGG CG- 3′

SEQ ID NO: 76: Antisense strand oligonucleotide S10

5′- TGC GAG ATA ATT TCT AAT ACC CTG AGA TGC ACG
ACA CGT TAT TGT TAC TCT- 3′

SEQ ID NO: 77: Antisense strand oligonucleotide S11

5′- ATA AAT TAA CAG CTT AGG TGC TTT ACC CGG CTT
CTG TTG ATA CCA- 3′

SEQ ID NO: 78: Antisense strand oligonucleotide S12

5′- CTA ATT GTC AAA GTA AAA TCC GTA CCG CTA CCT
GAA CCA CTA AAC CTA GAA GGC ACA CC- 3′

SEQ ID NO: 79: Antisense strand oligonucleotide S13

5′- ACA GTA ATA TGT TGC AAC GTC TTC TGG CTG TAA
TGA A -3′

SEQ ID NO: 80: Antisense strand oligonucleotide S14

5′- GGC CGC CTT GAT TTC GAC TTT AGT ACC TTG AGC
GAA-3′

SEQ ID NO: 81: Antisense strand oligonucleotide S15

5′-TCA GTT ACT CAC AAA TCT TCC TCT GAG ATC AAT TTT
TGT TCT CCG TGA TGG TGA TGG TGA TGA GC -3′

It is claimed:

1. An isolated human anti-TNF-α antibody, or antigen-binding portion thereof, containing at least one high-affinity V_{L} or V_{H} antibody chain that is effective, when substituted for the corresponding V_{L} or V_{H} chain of the anti-TNF-α scFv antibody having sequence SEQ ID NO: 1, to bind to human TNF-α with a K_{D} dissociation constant or a K_{off} rate constant that is at least 1.5 fold lower than that of the antibody having SEQ ID NO: 1, when determined under identical conditions.

2. The antibody of claim 1, whose V_{L} and V_{H} chains have the sequences identified by SEQ ID NOS 2 and 7, respectively, excluding SEQ ID NO: 1.

3. The antibody of claim 2, having at least one of the V_{L} CDR1, CDR2, and CDR3 regions whose sequence is identified by SEQ ID NOS: 3, 4 and 5, respectively, excluding SEQ ID NO: 1.

4. The antibody of claim 2, having at least one of the H_{L} CDR1, CDR2, and CDR3 regions whose a sequence is identified by SEQ ID NOS: 8, 9, and 10, respectively, excluding SEQ ID NO: 1.

5. An isolated human anti-TNF-α antibody, or antigen-binding portion thereof, having V_{L} and V_{H} antibody chains whose sequences are identified by SEQ ID NOS 2 and 7, respectively, excluding SEQ ID NO: 1.

6. The antibody of claim 5, having at least one of the V_{L} CDR1, CDR2, and CDR3 regions whose sequence is identified by SEQ ID NOS: 3, 4 and 5, respectively, excluding SEQ ID NO: 1.

7. The antibody of claim 5, having at least one of the H_{L} CDR1, CDR2, and CDR3 regions whose sequence is identified by SEQ ID NOS: 8, 9, and 10, respectively, excluding SEQ ID NO: 1.

8. A method of treating a condition that is aggravated by TNF-α activity in a mammalian subject, comprising
preparing a human anti-TNF-α antibody, or antigen-binding portion thereof, containing at least one high-affinity V_{L} or V_{H} antibody chain that is effective, when substituted for the corresponding V_{L} or V_{H} chain of the anti-TNF-α scFv antibody having sequence SEQ ID NO: 1, to bind to human TNF-α with a K_{D} dissociation constant or a K_{off} rate constant that is at least 1.5 lower than that of the antibody having SEQ ID NO: 1, when determined under identical conditions, and
administering said antibody to the subject, in an amount sufficient to improve the condition in the subject.

9. The method of claim 11, wherein the antibody prepared has V_{L} and V_{H} chains whose sequences are identified by SEQ ID NOS 2 and 7, respectively, excluding SEQ ID NO: 1.

10. The method of claim 9, wherein the antibody prepared has at least one of the V_{L} CDR1, CDR2, and CDR3 regions whose sequence is identified by SEQ ID NOS: 3, 4 and 5, respectively, excluding SEQ ID NO: 1.

11. The method of claim 9, wherein the antibody prepared has at least one of the HL CDR1, CDR2, and CDR3 regions whose sequence is identified by SEQ ID NOS: 8, 9, and 10, respectively, excluding SEQ ID NO: 1.

12. A method of generating human anti-TNF-α antibodies with enhanced binding affinity, comprising:
(i) using the amino-acid sequence variations contained in the SEQ ID NOS: 2 and 7 for the V_{H} and V_{L} CDRs, respectively, of the anti-TNF-α antibody defined by SEQ ID NO: 1, to construct a library of antibody coding sequences encoding both V_{H} and V_{L} chains of the antibody, and selected from the group consisting of:
(a) a combinatorial library of coding sequences that encode combinations of the V_{H} and V_{L} CDR amino-acid sequence variations contained in at least one of the V_{H} or V_{L} sequences specified in step (i),
(b) a walk-through mutagenesis library encoding, for at least one of said CDRs, the same amino acid substitution at multiple amino acid positions within that CDR, where the substituted amino acid corresponds to an amino acid variation found in at least one amino acid position of the V_{H} or V_{L} sequences specified in step (i), for that CDR, and
(c) a library of localized saturation mutation sequences encoding, for at least one of said CDRs, all 20 natural L-amino acids at an amino acid position that admits to a sequence variation in at least one V_{H} or V_{L} sequences specified in step (i),
(ii) expressing the library of coding sequences in an expression system in which the encoded anti-TNF-α antibodies are expressed in a selectable expression system, and
(iii) selecting those antibodies expressed in (iii) having the lowest K_{D} or EC50 k_{off} rate constants for human TNF-α.

13. The method of claim 12, wherein said constructing includes identifying amino acid positions that are invariant within one or more selected CDRs, and retaining the codons for the invariant amino acid in the library antibody coding sequences.

14. The method of claim 12, wherein the library of coding sequences is a combinatorial library of coding sequences constructed by (i) producing a primary library of coding sequence encoding antibodies a single amino acid variation contained in at least one of the V_{H} or V_{L} sequences specified in step (i), and (ii) shuffling the coding sequences in the primary library to produce a library of coding sequences having multiple amino acid variations contained in at least one of the V_{H} or V_{L} sequences specified in step (i).

15. The method of claim 12, wherein the library of coding sequences is a combinatorial library of coding sequences constructed by generating coding sequences having, at each amino acid variation position, codons for the wildtype amino acid and for each of the variant amino acids.

16. The method of claim 15, wherein the CDR coding regions of said library of coding sequences for the V_{L} chain have the sequences identified by SEQ ID NOS: 11-13, respectively.

17. The method of claim 15, wherein the CDR coding regions of said library of coding sequences for the V_{H} chain have the sequences identified by SEQ ID NOS: 14-16, respectively.

18. The method of claim 12, wherein the 15.the library of coding sequences are constructed to encode multiple positively charged amino acids in the CDR-L1 domain or multiple polar amino acids in the CDR-H3 domain.

19. The method of claim 12, wherein the expression system employed in carrying out step (ii) is a yeast expression system.

20. The method of claim 12, wherein the library of coding sequence encode scFv anti-TNF-α antibodies.

21. A library of combinatorial mutagenesis coding sequences whose CDR coding regions are selected from the group consisting of SEQ ID NOS: 11-16, for use in generating human anti-TNF-α antibodies having one or more of the amino acid substitutions in the V_{L}and V_{H} CDR regions of mutations identified in SEQ ID NOS: 2 and 7, respectively.


SEQUENCE LISTING (87 sequences)

SEQ ID NO: 1 - protein, 264 aa, Artificial sequence
  Recombinant D2E7 scFv antibody
MEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSGGGGSGGGGSGGGGSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKAAAHHHHHHGEQKLISEEDL

SEQ ID NO: 2 - protein, 127 aa, Artificial sequence
  Modified recombinant VL of D2E7
  VARIANT (24)..(24): X = R or H
  VARIANT (27)..(27): X = L, Q, R, K or Y
  VARIANT (28)..(28): X = G, E, R, S, Y or K
  VARIANT (29)..(29): X I, L or K
  VARIANT (29)..(29): X = I, L or K
  VARIANT (51)..(51): X = A or K
  VARIANT (52)..(52): X = L, S or Y
  VARIANT (53)..(53): X = X, A, K or T
  VARIANT (54)..(54): X = F, P or L
  VARIANT (55)..(55): X = Q, Y, K or L
  VARIANT (56)..(56): X = S, Q, H, R, K, N or P
  VARIANT (90)..(90): X = K or R
  VARIANT (92)..(92): X = N or D
  VARIANT (93)..(93): X = R, S, K, L, Q or D
  VARIANT (94)..(94): X = A, P or K
  VARIANT (95)..(95): X = P or Q
  VARIANT (96)..(96): X = Y or Q
  VARIANT (97)..(97): X = T or A
DIQMTQSPSSLSASVGDRVTITCXASXXXRNYLAWYQQKPGKAPKLLIYAXXXXXXGVPSRFSGSGSGTDFTLTISSLQPEDVATYYCQXYXXXXXXFGQGTKVEIKAAAHHHHHHGEQKLISEEDL

SEQ ID NO: 3 - protein, 11 aa, Artificial sequence
  Modified recombinant VL CDR1 sequence
  VARIANT (1)..(1): X = R or H
  VARIANT (4)..(4): X = L, Q, R, K or Y
  VARIANT (5)..(5): X = G, E, R, S, Y or K
  VARIANT (6)..(6): X = I, L or K
XASXXXRNYLA

SEQ ID NO: 4 - protein, 6 aa, Artificial sequence
  Synthetic VL CDR2 amino acid sequence
  VARIANT (1)..(1): X = A or K
  VARIANT (2)..(2): X = L, S or Y
  VARIANT (3)..(3): X = S, A, K or T
  VARIANT (4)..(4): X = F, P or L
  VARIANT (5)..(5): X = Q, Y, K or L
  VARIANT (6)..(6): X = S, Q, H, R, K, N or P
XXXXXX

SEQ ID NO: 5 - protein, 9 aa, Artificial sequence
  Synthetic VL CDR3 amino acid sequence
  VARIANT (2)..(2): X = K or R
  VARIANT (4)..(4): X = N or D
  VARIANT (5)..(5): X = R, S, K, L, Q or D
  VARIANT (6)..(6): X = A, P or K
  VARIANT (7)..(7): X = P or Q
  VARIANT (8)..(8): X = Y or Q
  VARIANT (9)..(9): X = T or A
QXYXXXXXX

SEQ ID NO: 6 - protein, 120 aa, Artificial sequence
  Recombinant D2E7 VH
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVS

SEQ ID NO: 7 - protein, 120 aa, Artificial sequence
  Modified recombinant VH sequence of D2E7
  VARIANT (31)..(31): X = D, Q, Y or H
  VARIANT (32)..(32): X = Y, S or H
  VARIANT (34)..(34): X = M or L
  VARIANT (52)..(52): X = T, S, I or A
  VARIANT (53)..(53): X = W or Y
  VARIANT (58)..(58): X = I, A or H
  VARIANT (59)..(59): X = D, K or S
  VARIANT (61)..(61): X = A, S or K
  VARIANT (63)..(63): X = S or P
  VARIANT (99)..(99): X = A, K, S or V
  VARIANT (100)..(100): X = S, K, Q, H, R or T
  VARIANT (101)..(101): X = Y, K, Q or H
  VARIANT (103)..(103): X = S or P
  VARIANT (105)..(105): X = A or S
  VARIANT (106)..(106): X = S, D or P
  VARIANT (107)..(107): X = S, Q or N
  VARIANT (108)..(108): X = L or H
  VARIANT (109)..(109): X = D, H, S or Q
  VARIANT (110)..(110): X = Y, N, S, L, Q or H
EVQLVESGGGLVQPGRSLRLSCAASGFTFDXXAXHWVRQAPGKGLEWVSAIXXNSGHXXYXDXVEGRFTISRDNAKNSLYLQMNSLRAEDTAVYYCAKXXXLXTXXXXXXWGQGTLVTVS

SEQ ID NO: 8 - protein, 5 aa, Artificial sequence
  Modified recombinant VH CDR1 sequence
  VARIANT (1)..(1): X = D, Q, Y or H
  VARIANT (2)..(2): X = Y, S or H
  VARIANT (4)..(4): X = M or L
XXAXH

SEQ ID NO: 9 - protein, 14 aa, Artificial sequence
  Modified recombinant VH CDR2 sequence
  VARIANT (1)..(1): X = T, S, I or A
  VARIANT (2)..(2): X = W or Y
  VARIANT (7)..(7): X = I, A or H
  VARIANT (8)..(8): X = D, K or S
  VARIANT (10)..(10): X = A, S or K
  VARIANT (12)..(12): X = S or P
XXNSGHXXYXDXVE

SEQ ID NO: 10 - protein, 12 aa, Artificial sequence
  Modified recombinant VH CDR3 sequence
  VARIANT (1)..(1): X = A, K, S or V
  VARIANT (2)..(2): X = S, K, Q, H, R or T
  VARIANT (3)..(3): X = Y, K, Q or H
  VARIANT (5)..(5): X = S or P
  VARIANT (7)..(7): X = A or S
  VARIANT (8)..(8): X = S, D or P
  VARIANT (9)..(9): X = S, Q or N
  VARIANT (10)..(10): X = L or H
  VARIANT (11)..(11): X = D, H, S or Q
  VARIANT (12)..(12): X = Y, N, S, L, Q or H
XXXLXTXXXXXX

SEQ ID NO: 11 - DNA, 33 bp, Artificial sequence
  Combinatorial coding sequence for VL CDR1
crtgcatcthrkrramwaagaaattatctcgca

SEQ ID NO: 12 - DNA, 21 bp, Artificial sequence
  Combinatorial coding sequence for VL CDR2
gccgcctmtwctttwcwamvk

SEQ ID NO: 13 - DNA, 27 bp, Artificial sequence
  Combinatorial coding sequence for VL CDR3
caaagatacratarasctccatataca

SEQ ID NO: 14 - DNA, 15 bp, Artificial sequence
  Combinatorial coding sequence for VH CDR1
bakymtgctmtgcat

SEQ ID NO: 15 - DNA, 42 bp, Artificial sequence
  Combinatorial coding sequence for VH CDR2
acatataattccggtcatattgattacgctgactctgttgag

SEQ ID NO: 16 - DNA, 36 bp, Artificial sequence
  Combinatorial coding sequence for VH CDR3
gtgmrktacttatcaacagcttctmrkctasakymk

SEQ ID NO: 17 - DNA, 795 bp, Artificial sequence
  Recombinant D2E7 scFv antibody
atggaagttcaattggtagaaagtggtgggggattagtgcaaccaggtagatctctaaggcttagctgtgctgcatctgggttcacctttgacgattatgctatgcattgggtccgacaagcgccaggaaaaggtctagagtgggtttctgcgataacatggaattccggtcatattgattacgctgactctgttgagggtagatttactatttcccgtgataatgctaagaactctttgtacttgcagatgaattctttaagagcagaggacaccgctgtatattactgtgcaaaggtgtcttacttatcaacagcttcttcgctagattattgggggcaaggcactctagtcactgttagttctggtggaggcggttctggtggaggcggttcgggtggcggaggttcagatatacaaatgacccaatcgccttctagcctttctgcaagtgttggagacagagtaacaataacgtgtcgtgcatctcagggtattagaaattatctcgcatggtatcaacagaagccgggtaaagcacctaagctgttaatttatgccgcctcaactttacaatctggtgtgccttctaggtttagtggttcaggtagcggtacggattttactttgacaattagttcattacagccagaagacgttgcaacatattactgtcaaagatacaatcgcgctccatatacattcggtcaaggtactaaagtcgaaatcaaggcggccgctcatcaccatcaccatcacggagaacaaaaattgatctcagaggaagatttgtga

SEQ ID NO: 18 - DNA, 32 bp, Artificial sequence
  Synthetic oligonucleotide
cgcggatccatggaagttcaattggtagaaag

SEQ ID NO: 19 - DNA, 30 bp, Artificial sequence
  Synthetic 3′ not I reverse flanking oligonucleotide
atggtggtgagcggccgccttgatttcgac

SEQ ID NO: 20 - DNA, 36 bp, Artificial sequence
  Synthetic FR5 anti-sense oligonucleotide
atcgtcaaaggtgaacccagatgcagcacagctaag

SEQ ID NO: 21 - DNA, 39 bp, Artificial sequence
  Synthetic FR1 sense oligonucleotide
atggaagttcaattggtagaaagtggtgggggattagtg

SEQ ID NO: 22 - DNA, 48 bp, Artificial sequence
  Synthetic FR2 anti-sense oligonucleotide
acccaggctgttcgcggtccttttccagatctcacccaaagacgctat

SEQ ID NO: 23 - DNA, 84 bp, Artificial sequence
  Synthetic CDR H2 LTM oligonucleotide
gtagagtgggtttctgcgataacttggaattctggtcatattgattatgctgattctgttgaaggtagatttactatttcccgt

SEQ ID NO: 24 - DNA, 84 bp, Artificial sequence
  Synthetic CDR H2 LTM leucine oligonucleotide
gtagagtgggtttctgcgatattgtggaattctggtcatattgattatgctgattctgttgaaggtagatttactatttcccgt

SEQ ID NO: 25 - DNA, 84 bp, Artificial sequence
  Synthetic CDR H2 LTM leucine oligonucleotide
gtagagtgggtttctgcgataactttgaattctggtcatattgattatgctgattctgttgaaggtagatttactatttcccgt

SEQ ID NO: 26 - DNA, 84 bp, Artificial sequence
  Synthetic CDR H2 LTM leucine oligonucleotide
gtagagtgggtttctgcgataacttggttgtctggtcatattgattatgctgattctgttgaaggtagatttactatttcccgt

SEQ ID NO: 27 - DNA, 84 bp, Artificial sequence
  Synthetic CDR H2 LTM leucine oligonucleotide
gtagagtgggtttctgcgataacttggaatttgggtcatattgattatgctgattctgttgaaggtagatttactatttcccgt

SEQ ID NO: 28 - DNA, 84 bp, Artificial sequence
  Synthetic CDR H2 LTM leucine oligonucleotide
gtagagtgggtttctgcgataacttggaattctttgcatattgattatgctgattctgttgaaggtagatttactatttcccgt

SEQ ID NO: 29 - DNA, 84 bp, Artificial sequence
  Synthetic CDR H2 LTM leucine oligonucleotide
gtagagtgggtttctgcgataacttggaattctggtttgattgattatgctgattctgttgaaggtagatttactatttcccgt

SEQ ID NO: 30 - DNA, 84 bp, Artificial sequence
  Synthetic CDR H2 LTM leucine oligonucleotide
gtagagtgggtttctgcgataacttggaattctggtcatttggattatgctgattctgttgaaggtagatttactatttcccgt

SEQ ID NO: 31 - DNA, 84 bp, Artificial sequence
  Synthetic CDR H2 LTM leucine oligonucleotide
gtagagtgggtttctgcgataacttggaattctggtcatattttgtatgctgattctgttgaaggtagatttactatttcccgt

SEQ ID NO: 32 - DNA, 84 bp, Artificial sequence
  Synthetic CDR H2 LTM leucine oligonucleotide
gtagagtgggtttctgcgataacttggaattctggtcatattgatttggctgattctgttgaaggtagatttactatttcccgt

SEQ ID NO: 33 - DNA, 84 bp, Artificial sequence
  Synthetic CDR H2 LTM leucine oligonucleotide
gtagagtgggtttctgcgataacttggaattctggtcatattgattatgctttgtctgttgaaggtagatttactatttcccgt

SEQ ID NO: 34 - DNA, 84 bp, Artificial sequence
  Synthetic CDR H2 LTM leucine oligonucleotide
gtagagtgggtttctgcgataacttggaattctggtcatattgattatgctgatttggttgaaggtagatttactatttcccgt

SEQ ID NO: 35 - DNA, 84 bp, Artificial sequence
  Synthetic CDR H2 LTM leucine oligonucleotide
gtagagtgggtttctgcgataacttggaattctggtcatattgattatgctgattctttggaaggtagatttactatttcccgt

SEQ ID NO: 36 - DNA, 84 bp, Artificial sequence
  Synthetic CDR H2 LTM leucine oligonucleotide
gtagagtgggtttctgcgataacttggaattctggtcatattgattatgctgattctgttttgggtagatttactatttcccgt

SEQ ID NO: 37 - DNA, 65 bp, Artificial sequence
  Synthetic FR4 anti-sense oligonucleotide
tcagttactcacaaatcttcctctgagatcaatttttgttctccgtgatggtgatggtgatgagc

SEQ ID NO: 38 - DNA, 84 bp, Artificial sequence
  Synthetic CDR H2 LTM aspartate oligonucleotide
gtagagtgggtttctgcgatagactggaattctggtcatattgattatgctgattctgttgaaggtagatttactatttcccgt

SEQ ID NO: 39 - DNA, 84 bp, Artificial sequence
  Synthetic CDR H2 WTM aspartate oligonucleotide
gtagagtgggtttctgcgatarmtkrkratkmtgrtsatrwtgatkatgmtgatkmtgwtgawggtagatttactatttcccgt

SEQ ID NO: 40 - skipped

SEQ ID NO: 41 - DNA, 51 bp, Artificial sequence
  Synthetic CDR-H1 LTM leucine oligonucleotide
ctgggttcacctttgacgcttatgctatgcattgggtccgacaagcgccag

SEQ ID NO: 42 - DNA, 51 bp, Artificial sequence
  Synthetic CDR-H1 LTM leucine oligonucleotide
ctgggttcacctttgacgatgctgctatgcattgggtccgacaagcgccag

SEQ ID NO: 43 - DNA, 51 bp, Artificial sequence
  Synthetic CDR-H1 LTM leucine oligonucleotide
ctgggttcacctttgacgattatgctatgcattgggtccgacaagcgccag

SEQ ID NO: 44 - DNA, 51 bp, Artificial sequence
  Synthetic CDR-H1 LTM leucine oligonucleotide
ctgggttcacctttgacgattatgctgctcattgggtccgacaagcgccag

SEQ ID NO: 45 - DNA, 51 bp, Artificial sequence
  Synthetic CDR-H1 LTM leucine oligonucleotide
ctgggttcacctttgacgattatgctatggcttgggtccgacaagcgccag

SEQ ID NO: 46 - DNA, 51 bp, Artificial sequence
  Synthetic CDR H1 beneficial mixed mutation oligonucleotide
ctgggttcacctttgacbakymtgctmtgcattgggtccgacaagcgccag

SEQ ID NO: 47 - DNA, 85 bp, Artificial sequence
  Synthetic CDR H2 beneficial mixed mutation oligonucleotide
ctagagtgggtttctgcgataacatataattccggtcatattgattacgctgactctgttgagggtagatttactattttcccgt

SEQ ID NO: 48 - DNA, 73 bp, Artificial sequence
  Synthetic CDR H3 beneficial mixed mutation oligonucleotide
gtatattactgtgcaaaggtgmrytacttatcaacagcttctmrkctasakymktgggggcaaggcactctag

SEQ ID NO: 49 - DNA, 72 bp, Artificial sequence
  Synthetic CDR L1 beneficial mixed mutation oligonucleotide
gacagagtaacaataacgtgtcrtgcatcthrkrramwaagaaattatctcgcatggtatcaacagaagccg

SEQ ID NO: 50 - DNA, 61 bp, Artificial sequence
  Synthetic CDR L2 beneficial mixed mutation oligonucleotide
cacctaagctgttaatttatgccgcctmtwctttwcwamvkggtgtgccttctaggtttag

SEQ ID NO: 51 - DNA, 69 bp, Artificial sequence
  Synthetic CDR L3 beneficial mixed mutation oligonucleotide
gacgttgcaacatattactgtcaaagatacratarasctccatatacattcggtcaaggtactaaagtc

SEQ ID NO: 52 - DNA, 39 bp, Artificial sequence; identical to SEQ ID NO: 21
  Synthetic sense strand oligonucleotide S1

SEQ ID NO: 53 - DNA, 40 bp, Artificial sequence
  Synthetic sense strand oligonucleotide S2
caaccaggtagatctctaaggcttagctgtgctgcatctg

SEQ ID NO: 54 - DNA, 48 bp, Artificial sequence
  Synthetic sense strand oligonucleotide S3
ggttcacctttgacgattatgctatgcattgggtccgacaagcgccag

SEQ ID NO: 55 - DNA, 51 bp, Artificial sequence
  Synthetic sense strand oligonucleotide S4
gaaaaggtctagagtgggtttctgcgataacatggaattccggtcatattg

SEQ ID NO: 56 - DNA, 47 bp, Artificial sequence
  Synthetic sense strand oligonucleotide S5
attacgctgactctgttgagggtagatttactatttcccgtgatatg

SEQ ID NO: 57 - DNA, 54 bp, Artificial sequence
  Synthetic sense strand oligonucleotide S6
ctaagaactctttgtacttgcagatgaattctttaagagcagaggacaccgctg

SEQ ID NO: 58 - DNA, 67 bp, Artificial sequence
  Synthetic sense strand oligonucleotide S7
tatattactgtgcaaaggtgtcttacttatcaacagcttcttcgctagattattgggggcaaggcac

SEQ ID NO: 59 - DNA, 63 bp, Artificial sequence
  Synthetic sense strand oligonucleotide S8
tctagtcactgttagttctggtggaggcggttctggtggaggcggttcgggtggcggaggttc

SEQ ID NO: 60 - DNA, 70 bp, Artificial sequence
  Synthetic sense strand oligonucleotide S9
agatatacaaatgacccaatcgccttctagcctttctgcaagtgttggagacagagtaacaataacgtgt

SEQ ID NO: 61 - DNA, 58 bp, Artificial sequence
  Synthetic sense strand oligonucleotide S10
cgtgcatctcagggtattagaaattatctcgcatggtatcaacagaagccgggtaaag

SEQ ID NO: 62 - DNA, 61 bp, Artificial sequence
  Synthetic sense strand oligonucleotide S11
cacctaagctgttaatttatgccgcctcaactttacaatctggtgtgccttctaggtttag

SEQ ID NO: 63 - DNA, 56 bp, Artificial sequence
  Synthetic sense strand oligonucleotide S12
tggttcaggtagcggtacggattttactttgacaattagttcattacagccagaag

SEQ ID NO: 64 - DNA, 66 bp, Artificial sequence
  Synthetic sense strand oligonucleotide S13
acgttgcaacatattactgtcaaagatacaatcgcgctccatatacattcggtcaaggtactaaag

SEQ ID NO: 65 - DNA, 51 bp, Artificial sequence
  Synthetic sense strand oligonucleotide S14
tcgaaatcaaggcggccgctcatcaccatcaccatcacggagaacaaaaat

SEQ ID NO: 66 - DNA, 31 bp, Artificial sequence
  Synthetic sense strand oligonucleotide S15
tgatctcagaggaagatttgtgagtaactga

SEQ ID NO: 67 - DNA, 45 bp, Artificial sequence
  Synthetic antisense strand oligonucleotide S1
ccttagagatctacctggttgcactaatcccccaccactttctac

SEQ ID NO: 68 - DNA, 33 bp, Artificial sequence
  Synthetic antisense strand oligonucleotide S2
gtcaaaggtgaacccagatgcagcacagctaag

SEQ ID NO: 69 - DNA, 42 bp, Artificial sequence
  Synthetic antisense strand oligonucleotide S3
agaaacccactctagaccttttcctggcgcttgtcggaccca

SEQ ID NO: 70 - DNA, 52 bp, Artificial sequence
  Synthetic antisense strand oligonucleotide S4
taccctcaacagagtcagcgtaatcaatatgaccggaattccatgttatcgc

SEQ ID NO: 71 - DNA, 54 bp, Artificial sequence
  Synthetic antisense strand oligonucleotide S5
aattcatctgcaagtacaaagagttcttagcattatcacgggaaatagtaaatc

SEQ ID NO: 72 - DNA, 41 bp, Artificial sequence
  Synthetic antisense strand oligonucleotide S6
ctttgcacagtaatatacagcggtgtcctctgctcttaaag

SEQ ID NO: 73 - DNA, 33 bp, Artificial sequence
  Synthetic antisense strand oligonucleotide S7
agaactaacagtgactagagtgccttgccccca

SEQ ID NO: 74 - DNA, 64 bp, Artificial sequence
  Synthetic antisense strand oligonucleotide S8
attgggtcatttgtatatctgaacctccgccacccgaaccgcctccaccagaaccgcctccacc

SEQ ID NO: 75 - DNA, 32 bp, Artificial sequence
  Synthetic antisense strand oligonucleotide S9
gtctccaacacttgcagaaaggctagaaggcg

SEQ ID NO: 76 - DNA, 51 bp, Artificial sequence
  Synthetic antisense strand oligonucleotide S10
tgcgagataatttctaataccctgagatgcacgacacgttattgttactct

SEQ ID NO: 77 - DNA, 45 bp, Artificial sequence
  Synthetic antisense strand oligonucleotide S11
ataaattaacagcttaggtgctttacccggcttctgttgatacca

SEQ ID NO: 78 - DNA, 59 bp, Artificial sequence
  Synthetic antisense strand oligonucleotide S12
ctaattgtcaaagtaaaatccgtaccgctacctgaaccactaaacctagaaggcacacc

SEQ ID NO: 79 - DNA, 37 bp, Artificial sequence
  Synthetic antisense strand oligonucleotide S13
acagtaatatgttgcaacgtcttctggctgtaatgaa

SEQ ID NO: 80 - DNA, 36 bp, Artificial sequence
  Synthetic antisense strand oligonucleotide S14
ggccgccttgatttcgactttagtaccttgaccgaa

SEQ ID NO: 81 - DNA, 65 bp, Artificial sequence; identical to SEQ ID NO: 37
  Synthetic antisense strand oligonucleotide S15

SEQ ID NO: 82 - protein, 5 aa, Artificial sequence
  Recombinant D2E7 scFv antibody
DYAMH

SEQ ID NO: 83 - protein, 12 aa, Artificial sequence
  Recombinant D2E7 scFv antibody
DYADSVEGRFTI

SEQ ID NO: 84 - protein, 14 aa, Artificial sequence
  Recombinant D2E7 scFv antibody
TWNSGHIDYADSVE

SEQ ID NO: 85 - protein, 28 aa, Artificial sequence
  Synthetic amino acid with D2E7 VH flanking CDR-H2 segment
LDWVSAITWNSGHIDYADSVEGRFTISR

SEQ ID NO: 86 - protein, 7 aa, Artificial sequence
  Recombinant VH framework 2 of D2E7 scFv antibody
LDWVSAI

SEQ ID NO: 87 - protein, 7 aa, Artificial sequence
  Recombinant VH framework 3 of D2E7 scFv antibody
GRFTISR